SEQUENCE LISTING (28 sequences)

SEQ ID NO: 1 - DNA, 1511 bp, Artificial sequence
  Synthetic polynucleotide
  misc_feature: 0-cut SA-GFP expression cassette
cctgcactaccagagctaactcactgacctcttctcttcctcccacagggcctcgagagatctggcagcggaggaagcggagctactaacttcagcctgctgaagcaggctggagacgtggaggagaaccctggacctaccggtatgcctgaaccctctaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttctttaaggatgacggcaactacaagacccgcgccgaggtgaagttcgagggcgacaccctggtgaaccgcatcgagctgaagggcatcgactttaaagaggacgggaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacctgcactaccagagctaactca

SEQ ID NO: 2 - DNA, 1511 bp, Artificial sequence
  Synthetic polynucleotide
  misc_feature: 1-cut SA-GFP expression cassette
cctatcctgtccctagtggccccctgacctcttctcttcctcccacagggcctcgagagatctggcagcggaggaagcggagctactaacttcagcctgctgaagcaggctggagacgtggaggagaaccctggacctaccggtatgcctgaaccctctaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttctttaaggatgacggcaactacaagacccgcgccgaggtgaagttcgagggcgacaccctggtgaaccgcatcgagctgaagggcatcgactttaaagaggacgggaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacctgcactaccagagctaactca

SEQ ID NO: 3 - DNA, 1511 bp, Artificial sequence
  Synthetic polynucleotide
  misc_feature: 2-cut SA-GFP expression cassette
cctatcctgtccctagtggccccctgacctcttctcttcctcccacagggcctcgagagatctggcagcggaggaagcggagctactaacttcagcctgctgaagcaggctggagacgtggaggagaaccctggacctaccggtatgcctgaaccctctaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttctttaaggatgacggcaactacaagacccgcgccgaggtgaagttcgagggcgacaccctggtgaaccgcatcgagctgaagggcatcgactttaaagaggacgggaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacctatcctgtccctagtggcccc

SEQ ID NO: 4 - DNA, 1892 bp, Artificial sequence
  Synthetic polynucleotide
  misc_feature: 0-cut SA-GFP scAAV
cctgcaggcagctgcgcgctcgctcgctcactgaggccgcccgggcaaagcccgggcgtcgggcgacctttggtcgcccggcctcagtgagcgagcgagcgcgcagagagggagtggacgcgtaggcctaagcttggtaccggatccactagtaacggccgccagtgtgctggaattcgcccttctttgagttagctctggtagtgcaggtagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagtcgaggctgatcagccatatgttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttcccgtcctctttaaagtcgatgcccttcagctcgatgcggttcaccagggtgtcgccctcgaacttcacctcggcgcgggtcttgtagttgccgtcatccttaaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgtccttagcgctggtgtacttggtgacggccttagtaccctcggacacggcgtgcttggccaactccccaggcagcagcaggcgcacggccgtctggatctccctggaggtgatggtcgagcgcttgttgtaatgcgccaggcgggaagcctcacctgcgatgcgctcgaaaatgtcgttcacaaacgaattcatgatgcccatggccttggacgaaatgccggtgtcagggtggacctgcttcagaaccttgtacacatagatggaatagctctccttgcggctgcgcttgcgcttcttgccgcctttcttctgcgccttagtcaccgccttcttggagccctttttcggggcgggagcagacttagagggttcaggcataccggtaggtccagggttctcctccacgtctccagcctgcttcagcaggctgaagttagtagctccgcttcctccgctgccagatctctcgaggccctgtgggaggaagagaagaggtcagtgagttagctctggtagtgcaggtaccaagggcgaattctgcagatatccatcacactggcttaattaagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 5 - DNA, 1895 bp, Artificial sequence
  Synthetic polynucleotide
  misc_feature: 2-cut SA-GFP scAAV
cctgcaggcagctgcgcgctcgctcgctcactgaggccgggcgaccaaaggtcgcccgacgcccgggctttgcccgcccggcctcagtgagcgagcgagcgcgcagagagggagtggacgcgtaggcctaagcttggtaccggatccactagtaacggccgccagtgtgctggaattcgcccttggtacctatcctgtccctagtggccccctgacctcttctcttcctcccacagggcctcgagagatctggcagcggaggaagcggagctactaacttcagcctgctgaagcaggctggagacgtggaggagaaccctggacctaccggtatgcctgaaccctctaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttctttaaggatgacggcaactacaagacccgcgccgaggtgaagttcgagggcgacaccctggtgaaccgcatcgagctgaagggcatcgactttaaagaggacgggaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacctatcctgtccctagtggccccaagcttaagggcgaattctgcagatatccatcacactggcttaattaagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 6 - DNA, 1613 bp, Artificial sequence
  Synthetic polynucleotide
  misc_feature: 2-cut SA-tNGFR scAAV
cctgcaggcagctgcgcgctcgctcgctcactgaggccgcccgggcaaagcccgggcgtcgggcgacctttggtcgcccggcctcagtgagcgagcgagcgcgcagagagggagtggacgcgtaggcctaagcttggtaccggatccactagtaacggccgccagtgtgctggaattcgcccttggtacctatcctgtccctagtggccccctgacctcttctcttcctcccacagggcctcgagagatctggcagcggaggaagcggagctactaacttcagcctgctgaagcaggctggagacgtggaggagaaccctggacctaccggtatgggggcaggtgccaccggccgcgccatggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttctgacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctacctatcctgtccctagtggccccaagcttaagggcgaattctgcagatatccatcacactggcttaattaagggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 3591 bp, Artificial sequence
  Synthetic polynucleotide
  misc_feature: HDR-TI SA-GFP ssAAV
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcagtcgataactataacggtcctaaggtagcgatttaaatacgcgctctcttaaggtagccccgggacgcgtcaattgagatctggatccggtacctgcactaccagagctaactcatgctttctctgaccagcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacagctgacctcttctcttcctcccacagggctctggcagcggaggaagcggagctactaactttagtctgcttaatcaagctggagacgtggaggagaaccctggacctaccggtatgcctgaaccctctaagtctgctcccgccccgaaaaagggctccaagaaggcggtgactaaggcgcagaagaaaggcggcaagaagcgcaagcgcagccgcaaggagagctattccatctatgtgtacaaggttctgaagcaggtccaccctgacaccggcatttcgtccaaggccatgggcatcatgaattcgtttgtgaacgacattttcgagcgcatcgcaggtgaggcttcccgcctggcgcattacaacaagcgctcgaccatcacctccagggagatccagacggccgtgcgcctgctgctgcctggggagttggccaagcacgccgtgtccgagggtactaaggccgtcaccaagtacaccagcgctaaggacatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttctttaaggatgacggcaactacaagacccgcgccgaggtgaagttcgagggcgacaccctggtgaaccgcatcgagctgaagggcatcgactttaaagaggacgggaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacatatggctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctgcactaccagagctaactcaaagcttctagatatcctctcttaaggtagcatcgagatttaaattagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 8 - DNA, 100 bp, Artificial sequence
  Synthetic polynucleotide
  misc_feature: AAVS1 sgRNA
ggggccacuagggacaggauguuuuagagcuagaaauagcaaguuaaaauaaggcuaguccguuaucaacuugaaaaaguggcaccgagucggugcuuuu

SEQ ID NO: 9 - DNA, 23 bp, Artificial sequence
  Synthetic polynucleotide
  misc_feature: Wild-type AAVS1 protospacer with PAM
ggggccactagggacaggattgg

SEQ ID NO: 10 - DNA, 20 bp, Artificial sequence
  Synthetic polynucleotide
  misc_feature: Primer 1 AAVS1-outsideF
cggaactctgccctctaacg

SEQ ID NO: 11 - DNA, 20 bp, Artificial sequence
  Synthetic polynucleotide
  misc_feature: Primer 2 AAVS1-outsideR
ctgggataccccgaagagtg

SEQ ID NO: 12 - DNA, 25 bp, Artificial sequence
  Synthetic polynucleotide
  misc_feature: AAVS1 TIDE-4R
cctctccatcctcttgctttctttg

SEQ ID NO: 13 - DNA, 23 bp, Artificial sequence
  Synthetic polynucleotide
  misc_feature: AAVS1 TIDE-4F
aactgcttctcctcttgggaagt

SEQ ID NO: 14 - DNA, 19 bp, Artificial sequence
  Synthetic polynucleotide
  misc_feature: IntAmp1
gatgggggtgttctgctgg

SEQ ID NO: 15 - DNA, 20 bp, Artificial sequence
  Synthetic polynucleotide
  misc_feature: IntAmp2
cgtaaacggccacaagttca

SEQ ID NO: 16 - DNA, 23 bp, Artificial sequence
  Synthetic polynucleotide
  misc_feature: AAVS1 DAP with PAM 1
ggggccactagggagaggatagg

SEQ ID NO: 17 - DNA, 23 bp, Artificial sequence
  Synthetic polynucleotide
  misc_feature: AAVS1 DAP with PAM 2
ggggccactagggccaggatagg

SEQ ID NO: 18 - DNA, 23 bp, Artificial sequence
  Synthetic polynucleotide
  misc_feature: AAVS1 DAP with PAM 3
ggggccactaggaacaggatagg

SEQ ID NO: 19 - DNA, 23 bp, Artificial sequence
  Synthetic polynucleotide
  misc_feature: AAVS1 DAP with PAM 4
gggaccactagggacaggatagg

SEQ ID NO: 20 - DNA, 23 bp, Artificial sequence
  Synthetic polynucleotide
  misc_feature: AAVS1 DAP with PAM 5
ggagccactagggacaggatagg

SEQ ID NO: 21 - DNA, 23 bp, Artificial sequence
  Synthetic polynucleotide
  misc_feature: AAVS1 DAP with PAM 6
gaggccactagggacaggatagg

SEQ ID NO: 22 - DNA, 23 bp, Artificial sequence
  Synthetic polynucleotide
  misc_feature: AAVS1 DAP with PAM 7
tgggccactagggacaggatagg

SEQ ID NO: 23 - DNA, 23 bp, Artificial sequence
  Synthetic polynucleotide
  misc_feature: AAVS1 DAP with PAM 8
caggccactagggacaggatagg

SEQ ID NO: 24 - DNA, 23 bp, Artificial sequence
  Synthetic polynucleotide
  misc_feature: AAVS1 DAP with PAM 9
taggccactagggacaggatagg

SEQ ID NO: 25 - DNA, 23 bp, Artificial sequence
  Synthetic polynucleotide
  misc_feature: AAVS1 DAP with PAM 10
ctagccactagggacaggatagg

SEQ ID NO: 26 - DNA, 23 bp, Artificial sequence
  Synthetic polynucleotide
  misc_feature: AAVS1 DAP with PAM 11
tatgccactagggacaggatagg

SEQ ID NO: 27 - DNA, 23 bp, Artificial sequence
  Synthetic polynucleotide
  misc_feature: AAVS1 DAP with PAM 12
gatgccactagggacaggatagg

SEQ ID NO: 28 - DNA, 23 bp, Artificial sequence
  Synthetic polynucleotide
  misc_feature: AAVS1 DAP with PAM 13
gtagccactagggacaggatagg

(19) United States
(12) Patent Application Publication
(10) Pub. No.: US 2022/0000933 A1
(43) Pub. Date: Jan. 6, 2022

(54) COMPOSITIONS AND METHODS FOR NHEJ-MEDIATED GENOME EDITING

(71) Applicants: CRISPR THERAPEUTICS AG, Zug (CH); BAYER HEALTHCARE LLC, Whippany, NJ (US)

(72) Inventors: Gregory J. Cost, South San Francisco, CA (US); Gene l. Uenishi, South San Francisco, CA (US)

(21) Appl. No.: 17/289,104

(22) PCT Filed: Oct. 30, 2019
(86) PCT No.: PCT/US19/58917

§ 371 Date: Apr. 27, 2021

Related U.S. Application Data
(60) Provisional application No. 62/752,959, filed on Oct. 30, 2018.

(51) Int. Cl. A61K 35/28; C12N 5/0789; C12N 15/90; C12N 15/11; C12N 9/22; C12N 15/86; A61P 37/04

(52) U.S. Cl. CPC A61K 35/28; C12N 5/0647; C12N 15/907; C12N 15/11; C12N 9/22; C12N 2510/00; A61P 37/04; C12N 2310/20; C12N 2800/80; C12N 2750/14143; C12N 2500/02; C12N 15/86

(57) ABSTRACT

The present application relates to compositions and methods for genome editing in cells by homology-independent mechanisms, in particular for genome editing in cells that lack the machinery necessary for repair by homology-dependent mechanisms.

### CROSS-REFERENCE TO RELATED APPLICATIONS

This application claims the benefit of priority to U.S. Provisional Patent Application Ser. No. 62/752,959, filed on Oct. 30, 2018, which is herein expressly incorporated by reference it its entirety, including any drawings.

### INCORPORATION OF THE SEQUENCE LISTING

The material in the accompanying Sequence Listing is hereby incorporated by reference into this application. The accompanying Sequence Listing text file, named 052984-52800WO_Sequence Listing.txt, was created on Oct. 28, 2019 and is 24,932 bytes.

### FIELD

The present disclosure relates to compositions and methods for genome editing in cells by homology-independent mechanisms. Further provided are methods of using such engineered cells, such as for the treatment of various diseases and conditions.

### BACKGROUND

Severe Combined Immunodeficiency is a rare genetic disorder most commonly caused by mutations in genes associated with lymphoid development, e.g., IL2Rg, RAG1, and IL7R, or genes associated with lymphocyte proliferation and metabolism, e.g., ADA and PNP. The phenotype of the disease is characterized by the lack of T- and/or B-cells, thus resulting in the inability to fight infections.

Currently, the most common cure for SCID is a bone marrow transplant for those who can find a close enough match. While 80% of patients who receive bone marrow transplant survive, they are relegated to life-long immunosuppression. Gene therapy has been attempted with gamma-retroviruses, but high incidences of leukemia have halted efforts.

The majority of SCID patients have mutations in specific loci, which are already part of a screen for SCID. Once identified, hematopoietic stem cells (HSCs) from the patients' bone marrow can be harvested for targeted genome editing of the SCID-causing mutated gene(s), either by gene replacement or gene correction. However, current techniques for gene editing in HSCs are unable to produce edited HSCs that persist long enough to produce an effective therapeutic benefit. Thus, improved gene editing techniques for use with HSCs are needed.

### SUMMARY

In one aspect, provided herein is a method for genome modification at a target locus in a hematopoietic stem cell (HSC), comprising: (a) introducing a nuclease or nucleic acid encoding the nuclease into the HSC, wherein the target locus comprises a first recognition sequence for the nuclease; and (b) introducing a double-stranded donor nucleic acid into the HSC, wherein the double-stranded nucleic acid comprises an exogenous nucleic acid sequence and is configured to be inserted into the target locus by a homology-independent mechanism. In some embodiments, the HSC is a long-term engrafting HSC (LT-HSC) or a SCID-repopulating cell. In some embodiments, the HSC is characterized by the following markers: Lin⁻ (lineage negative)/CD34⁺/CD38⁻/CD90⁺/CD45RA⁻. In some embodiments, Lin⁻ is characterized as one or more of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻.

In another aspect, provided herein is a method for genome modification at a target locus in a quiescent T cell, comprising: (a) introducing a nuclease or nucleic acid encoding the nuclease into the quiescent T cell, wherein the target locus comprises a first recognition sequence for the nuclease; and (b) introducing a double-stranded donor nucleic acid into the quiescent T cell, wherein the double-stranded nucleic acid comprises an exogenous nucleic acid sequence and is configured to be inserted into the target locus by a homology-independent mechanism. In some embodiments, the quiescent T cell is a non-activated T cell.

In another aspect, provided herein is a method for genome modification at a target locus in an HDR-deficient cell, comprising: (a) introducing a nuclease or nucleic acid encoding the nuclease into the HDR-deficient cell, wherein the target locus comprises a first recognition sequence for the nuclease; (b) introducing a double-stranded donor nucleic acid into the HDR-deficient cell, wherein the double-stranded nucleic acid comprises an exogenous nucleic acid sequence and is configured to be inserted into the target locus by a homology-independent mechanism, and (c) culturing the HDR-deficient cell for a time sufficient for integration of the double-stranded donor nucleic acid into the target locus, wherein steps (a), (b), and (c) are carried out such that the insertion efficiency for the double-stranded donor nucleic acid is at least about 4%. In some embodiments, the insertion efficiency for the double-stranded donor nucleic acid is at least about 8%.

In some embodiments, according to any of the methods for genome modification described above, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is cleaved at the second and/or third recognition sequence following introduction into the cell.

In some embodiments, according to any of the methods for genome modification described above, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the methods for genome modification described above, the nuclease is an RNA-guided endonuclease (RGEN), each of the recognition sequences for the nuclease in the target locus and double-stranded donor nucleic acid is a protospacer sequence, and the method further comprises introducing into the cell one or more gRNAs targeting one or more of the protospacer sequences. In some embodiments, the method comprises introducing into the cell a gRNA comprising a spacer targeting the protospacers in the target locus and the donor nucleic acid. In some embodiments, the protospacer in the target locus is in a forward orientation, the exogenous nucleic acid in the double-stranded donor nucleic acid is in a forward orientation, and the protospacers in the double-stranded donor nucleic acid are in a reverse orientation. In some embodiments, the protospacers in the target locus and the donor nucleic acid are the same. In some embodiments, at least one of the protospacers in the target locus and the donor nucleic acid is a delayed-action protospacer (DAP) incompletely matching the gRNA spacer. In some embodiments, the DAP i) is shorter in length than the gRNA spacer by at least about 1 nucleotide; and/or ii) comprises at least about 1 nucleotide mismatch with the gRNA spacer. In some embodiments, the protospacers in the donor nucleic acid are DAPs, and the protospacer in the target locus completely matches the gRNA spacer. In some embodiments, the double-stranded donor nucleic acid comprises two DAPs flanking the exogenous nucleic acid sequence. In some embodiments, the protospacers in the donor nucleic acid completely match the gRNA spacer, and the protospacer in the target locus is a DAP.

In some embodiments, according to any of the methods for genome modification described above employing an RGEN, the RGEN is a Cas9 nuclease.

In some embodiments, according to any of the methods for genome modification described above employing an RGEN, the method comprises introducing into the cell a ribonucleoprotein (RNP) comprising the RGEN and the one or more gRNAs.

In some embodiments, according to any of the methods for genome modification described above employing an RGEN, the method comprises introducing into the cell an mRNA encoding the RGEN.

In some embodiments, according to any of the methods for genome modification described above, the double-stranded donor nucleic acid is a double-stranded virus genome. In some embodiments, the double-stranded virus genome is an adenovirus genome, a lentivirus genome, or an adeno-associated virus (AAV) genome. In some embodiments, the AAV genome is a self-complementary AAV (scAAV) genome. In some embodiments, the scAAV genome is an scAAV6 genome.

In some embodiments, according to any of the methods for genome modification described above, the nuclease or nucleic acid encoding the nuclease is introduced into the cell before the donor nucleic acid is introduced into the cell. In some embodiments, the nuclease or nucleic acid encoding the nuclease is introduced into the cell no more than 1 hour before the donor nucleic acid is introduced into the cell. In some embodiments, the nuclease or nucleic acid encoding the nuclease is introduced into the cell no more than 5 minutes before the donor nucleic acid is introduced into the cell.

In some embodiments, according to any of the methods for genome modification described above, the cell is cultured under hypoxic conditions.

In some embodiments, according to any of the methods for genome modification described above, the cell is cultured no longer than about 48 hours prior to introducing the nuclease or nucleic acid encoding the nuclease and the donor nucleic acid into the cell. In some embodiments, the cell is cultured no longer than about 24 hours prior to introducing the nuclease or nucleic acid encoding the nuclease and the donor nucleic acid into the cell. In some embodiments, the cell is cultured no longer than about 2 hours prior to introducing the nuclease or nucleic acid encoding the nuclease and the donor nucleic acid into the cell.

In some embodiments, according to any of the methods for genome modification described above, the cell is cultured in the presence of a Notch ligand. In some embodiments, the Notch ligand is a Delta-like Notch ligand (DLL), Jagged-1, Jagged-2, or a conjugate thereof. In some embodiments, the Delta-like Notch ligand is DLL1, DLL3, or DLL4. In some embodiments, the Notch ligand is Fc-DLL1, Fc-DLL3, Fc-DLL4, Fc-Jagged-1, or Fc-Jagged-2.

In another aspect, provided herein is a method for engraftment in an individual of edited HSCs comprising an exogenous nucleic acid sequence inserted at a target locus, comprising: (a) carrying out a method for genome modification according to any of the embodiments described above on an input population of HSCs obtained from the individual to generate an output population of HSCs comprising a population of edited HSCs comprising the exogenous nucleic acid sequence inserted at the target locus; and (b) administering the population of edited HSCs to the individual such that the edited HSCs are engrafted in the individual (e.g., such that the edited HSCs persist in the individual for at least about any of 1, 2, 3, 4, 5, 6, 7, 8, 9, 10, 11, 12, 13, 14, 15, 16, or more weeks). In some embodiments, the amount of engraftment of edited HSC in the individual is the same or greater than the amount of engraftment of corresponding edited HSCs prepared using a homology-dependent mechanism. In some embodiments, the input population of HSCs obtained from the individual comprises a mixed population of HSCs comprising LT-HSCs and short-term engrafting HSCs (ST-HSCs), and the population of edited HSCs that engrafted comprise edited LT-HSCs. In some embodiments, administering the population of edited HSCs to the individual comprises administering the output population of HSCs to the individual.

In another aspect, provided herein is an engineered HSC comprising: (a) a target locus comprising a first recognition sequence for a nuclease; (b) the nuclease or a nucleic acid encoding the nuclease; and (c) a double-stranded donor nucleic acid comprising an exogenous nucleic acid sequence, wherein the double-stranded donor nucleic acid is configured to be inserted into the target locus by a homology-independent mechanism. In some embodiments, the HSC is an LT-HSC or a SCID-repopulating cell. In some embodiments, the HSC is characterized by the following markers: Lin⁻/CD34⁻/CD38⁻/CD90⁻/CD45RA⁻. In some embodiments, Lin⁻ is characterized as one or more of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻.

In another aspect, provided herein is an engineered HSC prepared by a method for genome modification at a target locus in a cell according to any of the embodiments described above. In some embodiments, the method comprises: (a) introducing a nuclease or nucleic acid encoding the nuclease into an input HSC, wherein the target locus comprises a first recognition sequence for the nuclease; and (b) introducing a double-stranded donor nucleic acid into the input HSC, wherein the double-stranded nucleic acid comprises an exogenous nucleic acid sequence and is configured to be inserted into the target locus by a homology-independent mechanism (e.g., NHEJ). In some embodiments, the HSC is an LT-HSC or a SCID-repopulating cell. In some embodiments, the HSC is characterized by the following markers: Lin⁻/CD34⁺/CD38⁻/CD90⁺/CD45RA⁻. In some embodiments, Lin⁻ is characterized as one or more of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻.

In another aspect, provided herein is an engineered quiescent T cell comprising: (a) a target locus comprising a first recognition sequence for a nuclease; (b) the nuclease or a nucleic acid encoding the nuclease; and (c) a double-stranded donor nucleic acid comprising an exogenous nucleic acid sequence, wherein the double-stranded donor nucleic acid is configured to be inserted into the target locus by a homology-independent mechanism. In some embodiments, the quiescent T cell is a non-activated T cell.

In another aspect, provided herein is an engineered quiescent T cell prepared by a method for genome modification at a target locus in a cell according to any of the embodiments described above. In some embodiments, the method comprises: (a) introducing a nuclease or nucleic acid encoding the nuclease into an input quiescent T cell, wherein the target locus comprises a first recognition sequence for the nuclease; and (b) introducing a double-stranded donor nucleic acid into the input quiescent T cell, wherein the double-stranded nucleic acid comprises an exogenous nucleic acid sequence and is configured to be inserted into the target locus by a homology-independent mechanism (e.g., NHEJ). In some embodiments, the quiescent T cell is a non-activated T cell.

In some embodiments, according to any of the engineered cells described above, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence.

In some embodiments, according to any of the engineered cells described above, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the engineered cells described above, the nuclease is an RNA-guided endonuclease (RGEN), each of the recognition sequences for the nuclease in the target locus and double-stranded donor nucleic acid is a protospacer sequence, and the engineered cell further comprises one or more gRNAs specific for each distinct recognition sequence for the nuclease in the target locus and double-stranded donor nucleic acid. In some embodiments, the engineered cell comprises a gRNA comprising a spacer targeting the protospacers in the target locus and the donor nucleic acid. In some embodiments, the protospacer in the target locus is in a forward orientation, the exogenous nucleic acid in the double-stranded donor nucleic acid is in a forward orientation, and the protospacers in the double-stranded donor nucleic acid are in a reverse orientation. In some embodiments, the protospacers in the target locus and the donor nucleic acid are the same. In some embodiments, at least one of the protospacers in the target locus and the donor nucleic acid is a delayed-action protospacer (DAP) incompletely matching the gRNA spacer. In some embodiments, the DAP i) is shorter in length than the gRNA spacer by at least about 1 nucleotide; and/or ii) comprises at least about 1 nucleotide mismatch with the gRNA spacer. In some embodiments, the protospacers in the donor nucleic acid are DAPs, and the protospacer in the target locus completely matches the gRNA spacer. In some embodiments, the double-stranded donor nucleic acid comprises two DAPs flanking the exogenous nucleic acid sequence. In some embodiments, the protospacers in the donor nucleic acid completely match the gRNA spacer, and the protospacer in the target locus is a DAP.

In some embodiments, according to any of the engineered cells comprising an RGEN described above, the RGEN is a Cas9 nuclease.

In some embodiments, according to any of the engineered cells comprising an RGEN described above, the engineered cell comprises a ribonucleoprotein (RNP) comprising the RGEN and the one or more gRNAs.

In some embodiments, according to any of the engineered cells comprising an RGEN described above, the engineered cell comprises an mRNA encoding the RGEN.

In some embodiments, according to any of the engineered cells described above, the double-stranded donor nucleic acid is a double-stranded virus genome. In some embodiments, the double-stranded virus genome is an adenovirus genome, a lentivirus genome, or an adeno-associated virus (AAV) genome. In some embodiments, the AAV genome is a self-complementary AAV (scAAV) genome. In some embodiments, the scAAV genome is an scAAV6 genome.

In another aspect, provided herein is a method of treating a disease or condition in a subject, wherein the disease or condition is characterized by deficient expression of a functional protein, comprising administering to the subject an engineered cell according to any of the embodiments described above, wherein the exogenous nucleic acid encodes a functional form of the protein that can be expressed in the engineered cell. In some embodiments, the disease or condition is SCID, and wherein the exogenous nucleic acid comprises a functional form of a gene mutated in the individual involved in lymphoid development or lymphocyte proliferation and/or metabolism. In some embodiments, the exogenous nucleic acid encodes a functional form of IL2Rg, RAG1, IL7R, ADA, or PNP. In some embodiments, the disease or condition is Gaucher disease, Fabry disease, mucopolysaccharidosis types I-IX, or adrenoleukodystrophy.

### BRIEF DESCRIPTION OF THE DRAWINGS

FIG. 1A shows flow cytometry results at day 14 (D14) for NHEJ-mediated targeted integration as determined by GFP expression in K562 cells treated with pCR2.1-TOPO-psSCRAM-SA-P2A-H2Bj-Venus-BGHpA-psSCRAM (0-cut vector plasmid) or pCR2.1-TOPO-psAAVS1-SA-P2A-H2Bj-Venus-BGHpA-psSCRAM (1-cut vector plasmid) at the indicated concentrations in the presence or absence of Cas9/gRNA RNP, where pCR2.1-TOPO is the vector plasmid backbone, psSCRAM is a scrambled protospacer sequence with the PAM, psAAVS1 is the same protospacer sequence and as the genomic DNA sequence the Cas9/gRNA RNP cleaves with the PAM, SA is the splice acceptor, P2A is a peptide cleavage signal sequence, H2Bj-Venus is a modified GFP fused to the histone H2B that stabilizes the GFP in the nucleus, and BGHpA is the bovine growth hormone polyadenylation sequence.

FIG. 1B shows quantification of the flow cytometry studies of FIG. 1A at day 7 (D7) and day 14 (D14).

FIG. 2 shows PCR analysis of targeted donor integration of 0-cut vector plasmid or 1-cut vector plasmid at the indicated concentrations in K562 cells in the presence or absence of Cas9/gRNA RNP.

FIG. 3A shows flow cytometry results for targeted integration in activated T cells by HDR, NHEJ, or both HDR and NHEJ.

FIG. 3B shows quantification of the flow cytometry studies of FIG. 3A.

FIG. 4 shows flow cytometry results for the CD4/CD8 distribution of activated T cells with targeted integration by HDR, NHEJ, or both HDR and NHEJ by utilizing an AAV6 delivering an HDR donor, an scAAV6 2-cut NHEJ donor, or both respectively.

FIG. 5A shows flow cytometry results for targeted integration in non-activated T cells by HDR, NHEJ, or both HDR and NHEJ by utilizing an AAV6 delivering an HDR donor, an scAAV6 2-cut NHEJ donor, or both respectively.

FIG. 5B shows quantification of the flow cytometry studies of FIG. 5A.

FIG. 5C shows flow cytometry results for the CD4/CD8 distribution of non-activated T cells with targeted integration by NHEJ.

FIG. 6 shows flow cytometry results for NHEJ-mediated targeted integration in human CD34⁺ cells using Protocol 1 or Protocol 2.

FIG. 7A shows quantification of flow cytometry results for NHEJ-mediated targeted integration in human CD34⁺ cells using Protocol 1 or Protocol 2.

FIG. 7B shows quantification of flow cytometry results for NHEJ-mediated targeted integration in human CD34⁺ cells from two different donors with scAAV6 2-cut donors.

FIG. 8A shows absolute cellularity for NHEJ-mediated targeted integration in human CD34⁺ cells using Protocol 1 or Protocol 2.

FIG. 8B shows absolute cellularity for NHEJ-mediated targeted integration in human CD34⁺ cells from two different donors with scAAV6 2-cut donors.

FIG. 9A shows flow cytometry results at day 2 (D2), day 3 (D3), day 4 (D4), and day 5 (D5) for NHEJ-mediated targeted integration in human CD34⁺ cells using Protocol 1 or Protocol 2 when edited on D1.

FIG. 9B shows quantification of flow cytometry results at D2, D3, D4, and D5 for NHEJ-mediated targeted integration in human CD34⁺ cells using Protocol 1 or Protocol 2 when edited on D1.

FIG. 9C shows absolute cellularity at D2, D3, D4, and D5 for NHEJ-mediated targeted integration in human CD34⁺ cells using Protocol 1 or Protocol 2 when edited on D1.

FIG. 9D shows relative cellularity at D2, D3, D4, and D5 for NHEJ-mediated targeted integration in human CD34⁺ cells using Protocol 1 or Protocol 2 when edited on D1.

FIG. 10A shows flow cytometry results at D2, D3, D4, and D5 for NHEJ-mediated targeted integration in human CD34⁺ cells when edited on D1 in SFEM II medium.

FIG. 10B shows flow cytometry results at D2, D3, D4, and D5 for NHEJ-mediated targeted integration in human CD34⁺ cells at D1 when edited on SCGM medium.

FIG. 11A shows quantification of flow cytometry results at D2, D3, D4, and D5 for NHEJ-mediated targeted integration in human CD34⁺ cells when edited on D1 with scAAV6 2-cut donor under hypoxic or normoxic conditions.

FIG. 11B shows absolute cellularity at D2, D3, D4, and D5 for NHEJ-mediated targeted integration in human CD34⁺ cells when edited on D1 with scAAV6 2-cut donor under hypoxic or normoxic conditions.

FIG. 11C shows relative cellularity at D2, D3, D4, and D5 for NHEJ-mediated targeted integration in human CD34⁺ cells when edited on D1 with scAAV6 2-cut donor under hypoxic or normoxic conditions.

FIG. 11D shows the ratio of CD34⁺:CD34⁻ cells in GFP⁺ and eGFP⁻ populations at D2, D3, D4, and D5 for NHEJ-mediated targeted integration in human CD34⁺ cells when edited on D1 with scAAV6 2-cut donor under hypoxic or normoxic conditions.

FIG. 12A shows quantification of flow cytometry results at D2, D3, D4, and D5 for NHEJ-mediated targeted integration in human CD34⁺ cells when edited on D1 or D2 with scAAV6 2-cut donor.

FIG. 12B shows absolute cellularity at D2, D3, D4, and D5 for NHEJ-mediated targeted integration in human CD34⁺ cells when edited on D1 or D2 with scAAV6 2-cut donor.

FIG. 12C shows relative cellularity at D2, D3, D4, and D5 for NHEJ-mediated targeted integration in human CD34⁺ cells when edited on D1 or D2 with scAAV6 2-cut donor.

FIG. 13 shows quantification of flow cytometry results and relative cellularity at D2, D3, and D4 for NHEJ-mediated targeted integration in human CD34⁺ cells when edited at H1 (hour 1) or D1 with scAAV6 2-cut donor.

FIG. 14A shows quantification of flow cytometry results at D2 for NHEJ-mediated targeted integration in human CD34⁺ cells when edited on D1 using RNP Lonza 4-D nucleofection protocol DZ-100 or RNP Lonza 4-D nucleofection protocol CA-137.

FIG. 14B shows absolute cellularity at D2 for NHEJ-mediated targeted integration in human CD34⁺ cells when edited on D1 using RNP Lonza 4-D nucleofection protocol DZ-100 or RNP Lonza 4-D nucleofection protocol CA-137.

FIG. 14C shows relative cellularity at D2 for NHEJ-mediated targeted integration in human CD34⁺ cells when edited on D1 using RNP Lonza 4-D nucleofection protocol DZ-100 or RNP Lonza 4-D nucleofection protocol CA-137.

FIG. 15 shows quantification of flow cytometry results, absolute cellularity, and relative cellularity for NHEJ-mediated targeted integration in human CD34⁺ cells when edited on D1 using SpyFi Cas9 or GeneArt Cas9.

FIG. 16 shows quantification of flow cytometry results, absolute cellularity, and relative cellularity for NHEJ-mediated targeted integration in human CD34⁺ cells when edited on D1 using low (+) or high (+++) amounts of RNP and low (+) or high (+++) amounts of AAV.

FIG. 17 shows PCR analysis of targeted donor integration of 2-cut donors delivered by Ad5/35 at 5000 vp/cells in CD34⁺ cells in the presence or absence of Cas9/gRNA RNP.

FIG. 18A shows quantification of flow cytometry results at D2 for NHEJ-mediated targeted integration in human CD34⁺ cells using a scAAV6 0-cut donor cultured on Tenascin C (TenC), Fc-DLL1 (DLL1), or Tenascin C+Fc-DLL1 coating, or no coating.

FIG. 18B shows absolute cellularity of CD34⁺/CD90⁺/CD38⁻/CD45RA⁻ cells at D1, D2, and D3 for NHEJ-mediated targeted integration in human CD34⁺ cells using a scAAV6 0-cut donor cultured on Tenascin C (TenC), Fc-DLL1 (DLL1), or Tenascin C+Fc-DLL1 coating, or no coating.

FIG. 19 shows quantification of flow cytometry results for hCD45⁺ cells (top panels) and GFP⁺ cells (bottom panels) at weeks 0, 6, 9, 13, and 16 from peripheral blood of mice injected at D2 with human CD34⁺ cells edited at D1 by NHEJ-mediated targeted integration of scAAV6 0-cut or scAAV6 2-cut.

FIG. 20A shows quantification of flow cytometry results for hCD45⁺ cells (top panel) and GFP⁺ cells (bottom panel) at 16 weeks from bone marrow of mice injected at D2 with human CD34⁺ cells edited at D1 by NHEJ-mediated targeted integration of scAAV6 0-cut or scAAV6 2-cut.

FIG. 20B shows the relative amount of hCD34⁺, hCD3⁺, hCD33⁺, hCD19⁺, and other hCD45⁺ cells as a percent of total CD45⁺ cells from bone marrow of mice at 16 weeks following injection at D1 with human CD34⁺ cells edited at D1 by NHEJ-mediated targeted integration of scAAV6 0-cut or scAAV6 2-cut.

FIG. 21 shows quantification of flow cytometry results for GFP⁺/CD34⁺ cells at D2 of human CD34⁺ cells edited at D1 of H1 (Fresh Thaw) by NHEJ-mediated targeted integration of scAAV6 0-cut or scAAV6 2-cut.

FIG. 22A shows quantification of flow cytometry results for hCD45⁺ cells at 8 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 22B shows the relative amount of hCD3⁺, hCD33⁺, hCD19⁺, and other hCD45⁺ cells as a percent of total CD45⁺ cells from peripheral blood of mice at 8 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 22C shows quantification of flow cytometry results for GFP⁺ cells as a percent of hCD45⁺ cells at 8 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 22D shows quantification of flow cytometry results for GFP⁺ cells as a percent of total CD45⁺ cells at 8 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 23A shows quantification of flow cytometry results for hCD45⁺ cells at 10 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 23B shows the relative amount of hCD3⁺, hCD33⁺, hCD19⁺, and other hCD45⁺ cells as a percent of total CD45⁺ cells from peripheral blood of mice at 10 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 23C shows quantification of flow cytometry results for GFP⁺ cells as a percent of hCD45⁺ cells at 10 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 23D shows quantification of flow cytometry results for GFP⁺ cells as a percent of total CD45⁺ cells at 10 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 24A shows quantification of flow cytometry results for hCD45⁺ cells at 12 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 24B shows the relative amount of hCD3⁺, hCD33⁺, hCD19⁺, and other hCD45⁺ cells as a percent of total CD45⁺ cells from peripheral blood of mice at 12 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 24C shows quantification of flow cytometry results for GFP⁺ cells as a percent of hCD45⁺ cells at 12 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 24D shows quantification of flow cytometry results for GFP⁺ cells as a percent of total CD45⁺ cells at 12 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 25A shows quantification of flow cytometry results for hCD45⁺ cells at 14 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 25B shows the relative amount of hCD3⁺, hCD33⁺, hCD19⁺, and other hCD45⁺ cells as a percent of total CD45⁺ cells from peripheral blood of mice at 14 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 25C shows quantification of flow cytometry results for GFP⁺ cells as a percent of hCD45⁺ cells at 14 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 25D shows quantification of flow cytometry results for GFP⁺ cells as a percent of total CD45⁺ cells at 14 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 26A shows quantification of flow cytometry results for hCD45⁺ cells at 16 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 26B shows the relative amount of hCD3⁺, hCD33⁺, hCD19⁺, and other hCD45⁺ cells as a percent of total CD45⁺ cells from peripheral blood of mice at 16 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 26C shows quantification of flow cytometry results for GFP⁺ cells as a percent of hCD45⁺ cells at 16 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 26D shows quantification of flow cytometry results for GFP⁺ cells as a percent of total CD45⁺ cells at 16 weeks from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 27A shows quantification of flow cytometry results for hCD45⁺ cells at 16 weeks from bone marrow of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 27B shows the relative amount of hCD3⁺, hCD33⁺, hCD19⁺, and other hCD45⁺ cells as a percent of total CD45⁺ cells from bone marrow of mice at 16 weeks following i) injection at D1 with human CD34⁺ cells edited at D1 by NHEJ-mediated targeted integration of scAAV6 0-cut or scAAV6 2-cut or ii) injection at H2 with human CD34⁺ cells edited at H1 by NHEJ-mediated targeted integration of scAAV6 2-cut (Fresh Thaw).

FIG. 28A shows quantification of flow cytometry results for GFP⁺ cells as a percent of CD34⁺ cells at 16 weeks from bone marrow of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 28B shows quantification of flow cytometry results for GFP⁺ cells as a percent of hCD45⁺ cells at 16 weeks from bone marrow of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 28C shows quantification of flow cytometry results for GFP⁺ cells as a percent of total CD45⁺ cells at 16 weeks from bone marrow of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 29A shows the time course of hCD45⁺ blood cells from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 29B shows the time course of GFP⁺ cells as a percent of hCD45⁺ blood cells from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

FIG. 29C shows the time course of GFP⁺ cells as a percent of total CD45⁺ blood cells from peripheral blood of mice i) injected at D2 with human CD34⁺ cells edited at D1 by NHEJ-mediated targeted integration of scAAV6 0-cut or scAAV6 2-cut or ii) injected at H2 with human CD34⁺ cells edited at H1 by NHEJ-mediated targeted integration of scAAV6 2-cut (Fresh Thaw).

### DETAILED DESCRIPTION

Applicants have discovered systems and methods for genome modification at a target locus in a cell that performs homology directed repair (HDR) poorly. In particular, Applicants have demonstrated improved editing in long-term engrafting hematopoietic stem cells (LT-HSCs) and other quiescent cells, including quiescent (resting) T cells, as well as methods of making and using the engineered cells, and compositions useful for such methods.

Previous data have shown that HSCs that undergo HDR are those that have lost their long-term engraftment potential, rendering the corrected HSCs incapable of permanent therapeutic correction of disease. Conversely, HSCs retaining long-term engrafting potential are non-dividing and thus do not undergo HDR efficiently. By contrast, non-homologous end-joining (NHEJ) is a DNA double-stranded break (DSB) repair mechanism that operates in all cell types, including quiescent cells such as LT-HSCs. Applicants discovered that this pathway can be manipulated to mediate targeted integration of a transgene in quiescent LT-HSCs and quiescent T cells that allows for long-term engraftment of the edited cells and persistent expression of the transgene. For example, human CD34⁺ cells were edited using a CRISPR/Cas9 system for targeted integration of a GFP expression cassette into an endogenous PPP1R12C locus employing a double-stranded donor template for NHEJ-mediated integration with CRISPR/Cas9 recognition sites for in vivo donor cleavage. In mice injected with the edited cells, the amount of GFP⁺ cells as a fraction of hCD45⁺ cells from peripheral bleeds remained steady through week 16 following injection, demonstrating that the CRISPR/Cas9 system was capable of editing LT-HSCs and that the edited LT-HSCs were able to engraft and persist for a sufficient amount of time to be useful in therapeutic applications. With this platform technology, targeted gene correction or insertion of a heterologous nucleic acid can be performed in LT-HSCs without loss of their engraftment potential, enabling the use of these edited HSCs in therapeutic applications.

### Definitions

Unless defined otherwise, all technical and scientific terms used herein have the same meaning as commonly understood by one of ordinary skill in the art to which the disclosure pertains. All patents, applications, published applications and other publications referenced herein are expressly incorporated by reference in their entireties unless stated otherwise. In the event that there are a plurality of definitions for a term herein, those in this section prevail unless stated otherwise.

As used herein, “a” or “an” may mean one or more than one.

“About” has its plain and ordinary meaning when read in light of the specification, and may be used, for example, when referring to a measurable value and may be meant to encompass variations of ±20% or ±10%, more preferably ±5%, even more preferably ±1%, and still more preferably ±0.1% from the specified value.

As used herein, “protein sequence” refers to a polypeptide sequence of amino acids that is the primary structure of a protein. As used herein “upstream” refers to positions 5′ of a location on a polynucleotide, and positions toward the N-terminus of a location on a polypeptide. As used herein “downstream” refers to positions 3′ of a location on nucleotide, and positions toward the C-terminus of a location on a polypeptide. Thus, the term “N-terminal” refers to the position of an element or location on a polynucleotide toward the N-terminus of a location on a polypeptide.

“Nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also comprises so-called “peptide nucleic acids,” which comprise naturally-occurring or modified nucleic acid bases attached to a polyamide backbone. Nucleic acids can be either single stranded or double-stranded. In some embodiments, a nucleic acid sequence encoding a fusion protein is provided. In some embodiments, the nucleic acid is RNA or DNA.

“Coding for” or “encoding” are used herein, and refers to the property of specific sequences of nucleotides in a polynucleotide, such as a gene, a cDNA, or an mRNA, to serve as templates for synthesis of other macromolecules such as a defined sequence of amino acids. Thus, a gene codes for a protein if transcription and translation of mRNA corresponding to that gene produces the protein in a cell or other biological system.

A “nucleic acid sequence coding for a polypeptide” comprises all nucleotide sequences that are degenerate versions of each other and that code for the same amino acid sequence. In some embodiments, a nucleic acid is provided, wherein the nucleic acid encodes a fusion protein.

“Vector,” “expression vector,” “donor,” or “construct” is a nucleic acid used to introduce heterologous nucleic acids into a cell that has regulatory elements to provide expression of the heterologous nucleic acids in the cell. Vectors include but are not limited to plasmid, minicircles, yeast, and viral genomes. In some embodiments, the vectors are plasmid, minicircles, yeast, or viral genomes. In some embodiments, the vector is a viral vector. In some embodiments, the viral vector is a lentivirus. In some embodiments, the vector is an adeno-associated viral (AAV) vector. In some embodiments, the vector is for protein expression in a bacterial system such as E. coli. As used herein, the term “expression,” or “protein expression” refers to refers to the translation of a transcribed RNA molecule into a protein molecule. Protein expression may be characterized by its temporal, spatial, developmental, or morphological qualities as well as by quantitative or qualitative indications. In some embodiments, the protein or proteins are expressed such that the proteins are positioned for dimerization in the presence of a ligand.

As used herein, the term “regulatory element” refers to a DNA molecule having gene regulatory activity, e.g., one that has the ability to affect the transcription and/or translation of an operably linked transcribable DNA molecule. Regulatory elements such as promoters, leaders, introns, and transcription termination regions are DNA molecules that have gene regulatory activity and play an integral part in the overall expression of genes in living cells. Isolated regulatory elements, such as promoters, that function in plants are therefore useful for modifying plant phenotypes through the methods of genetic engineering.

As used herein, the term “operably linked” refers to a first molecule joined to a second molecule, wherein the molecules are so arranged that the first molecule affects the function of the second molecule. The two molecules may be part of a single contiguous molecule and may be adjacent. For example, a promoter is operably linked to a transcribable DNA molecule if the promoter modulates transcription of the transcribable DNA molecule of interest in a cell.

A “promoter” is a region of DNA that initiates transcription of a specific gene. The promoters can be located near the transcription start site of a gene, on the same strand and upstream on the DNA (the 5′region of the sense strand). The promoter can be a conditional, inducible or a constitutive promoter. The promoter can be specific for bacterial, mammalian or insect cell protein expression. In some embodiments, wherein a nucleic acid encoding a fusion protein is provided, the nucleic acid further comprises a promoter sequence. In some embodiments, the promoter is specific for bacterial, mammalian or insect cell protein expression. In some embodiments, the promoter is a conditional, inducible or a constitutive promoter.

As used herein, a “subject” refers to an animal that is the object of treatment, observation or experiment. “Animal” comprises cold- and warm-blooded vertebrates and invertebrates such as fish, shellfish, reptiles and, in particular, mammals. “Mammal” comprises, without limitation, mice, rats, rabbits, guinea pigs, dogs, cats, sheep, goats, cows, horses, primates, such as monkeys, chimpanzees, and apes, and, in particular, humans. In some alternative, the subject is human.

### Systems for NHEJ-Mediated Genome Editing

In one aspect, provided herein is a system for generating engineered cells (e.g., engineered hematopoietic stem cells, such as LT-HSCs) comprising targeted integration of an exogenous nucleic acid, wherein the exogenous nucleic acid is inserted into a target locus by non-homologous end joining (NHEJ). The system comprises a) a nuclease or nucleic acid encoding the nuclease, wherein the nuclease is capable of mediating genome editing at a target locus comprising a first recognition sequence for the nuclease; and b) a double-stranded donor nucleic acid, wherein the double-stranded donor nucleic acid comprises an exogenous nucleic acid sequence and is configured to be inserted into the target locus by NHEJ. In some embodiments, the system further comprises the cell to be edited. Also provided are, inter alia, systems for treating a subject having or suspected of having a disorder or health condition associated with a functional target protein deficit, employing ex vivo genome editing. For example, in some embodiments, the subject has a disease resulting from a functional deficit of the target protein (e.g., deficient expression and/or activity of the target protein) in target cells, and the system is capable of mediating genome editing in the target cells or progenitors thereof that allows for sufficient expression of the target protein or a functional derivative thereof in the genome-edited cells or their progeny in the subject such that the disease is treated. In some embodiments, the target cells or progenitors thereof are edited ex vivo and administered to the subject.

In some embodiments, according to any of the systems described herein comprising a double-stranded donor nucleic acid, the double-stranded donor nucleic acid further comprises a second recognition sequence for the nuclease flanking a first end of the exogenous nucleic acid sequence. For example, in some embodiments the second recognition sequence for the nuclease is immediately adjacent to the first end of the exogenous nucleic acid sequence or is about 5 or more (such as about any of 10, 20, 50, 100, 200, 500, 1000, or more) bases away from the first end of the exogenous nucleic acid sequence. In some embodiments, the double-stranded donor nucleic acid further comprises a third recognition sequence flanking a second end of the exogenous nucleic acid sequence. For example, in some embodiments the third recognition sequence for the nuclease is immediately adjacent to the second end of the exogenous nucleic acid sequence or is about 5 or more (such as about any of 10, 20, 50, 100, 200, 500, 1000, or more) bases away from the second end of the exogenous nucleic acid sequence. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the systems described herein comprising a double-stranded donor nucleic acid, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the systems described herein comprising a nuclease or nucleic acid encoding the nuclease, the nuclease is an RNA-guided endonuclease (RGEN). In some embodiments, the RGEN is a Cas9 nuclease. In some embodiments, the system further comprises a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the system comprises a ribonucleoprotein (RNP) comprising the RGEN and the gRNA. In some embodiments, the system comprises a nucleic acid encoding the RGEN. In some embodiments, the nucleic acid encoding the RGEN is an mRNA or a plasmid.

In some embodiments, according to any of the systems described herein comprising an RGEN or nucleic acid encoding the RGEN, each of the recognition sequences for the nuclease in the target locus and double-stranded donor nucleic acid is a protospacer sequence. In some embodiments, the system further comprises one or more gRNAs targeting one or more of the protospacer sequences. In some embodiments, each of the protospacers in the target locus and double-stranded donor nucleic acid are the same, and the system comprises one gRNA targeting the protospacer sequence.

The systems described herein in some embodiments comprise i) a first nucleic acid comprising a first protospacer and ii) a gRNA comprising a spacer, wherein the first protospacer is an incomplete match to the spacer, and wherein the degree to which the first protospacer matches the spacer is sufficient to allow for modification of the first nucleic acid at the first protospacer. Such a protospacer in relation to a corresponding gRNA is also referred to herein as a “delayed-action protospacer” or “DAP.” Such systems allow for temporal control of RGEN-mediated cleavage of the first nucleic acid at the first protospacer by varying the degree of matching between the DAP and the spacer. In some embodiments, the DAP is shorter in length than the spacer by at least about 1 (such as at least about any of 2, 3, or more) nucleotide. In some embodiments, the DAP comprises at least about 1 (such as at least about any of 2, 3, or more) nucleotide mismatch with the spacer. For example, the gRNA may comprise a spacer from the polynucleotide sequence of SEQ ID NO: 8, and the first protospacer may comprise a protospacer from the polynucleotide sequence of any one of SEQ ID NOs: 16-28. In some embodiments, the system further comprises a second nucleic acid comprising a second protospacer, wherein the second protospacer is a complete match to the spacer. For example, the gRNA may comprise a spacer from the polynucleotide sequence of SEQ ID NO: 8, and the second protospacer may comprise a protospacer from the polynucleotide sequence of SEQ ID NO: 9. Such systems allow for different temporal profiles of RGEN-mediated cleavage of the first and second nucleic acids at their respective protospacers using a single gRNA, with quicker onset of cleavage for the second nucleic acid at the second protospacer that completely matches the gRNA spacer as compared to onset of cleavage for the first nucleic acid at the first protospacer that incompletely matches the gRNA spacer.

The presence of DAPs in nucleic acids, such as a target nucleic acid or donor template, to facilitate temporal control of target site cleavage has several advantages. For one thing, the presence of a DAP in such nucleic acids allows temporal control of target site cutting at multiple loci without the need for multiple gRNAs for each loci administered at different times. This in turn permits genome editing systems to be packaged in comparatively less space, and the use of fewer gRNAs reduces the chance of off-target effects. Further advantages of the embodiments of this disclosure will be evident to those of skill in the art.

In some embodiments, according to any of the systems described herein, the system comprises a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. For example, in some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the system further comprises a cell comprising the target locus. In some embodiments, the cell is deficient in the machinery necessary for homology directed repair (HDR). In some embodiments, the cell is a hematopoietic stem cell (HSC), such as a long-term engrafting hematopoietic stem cell (LT-HSC). In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell.

In some embodiments, according to any of the systems described herein, the system comprises a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the system further comprises a cell comprising the target locus.

In some embodiments, according to any of the systems described herein, the system comprises a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the system further comprises a cell comprising the target locus.

### Methods of NHEJ-Mediated Genome Editing

In one aspect, provided herein is a method for genome modification at a target locus in a cell, comprising: (a) introducing a nuclease or nucleic acid encoding the nuclease into the cell, wherein the target locus comprises a first recognition sequence for the nuclease; and (b) introducing a double-stranded donor nucleic acid into the cell, wherein the double-stranded nucleic acid comprises an exogenous nucleic acid sequence and is configured to be inserted into the target locus by NHEJ.

In some embodiments, according to any of the methods for genome modification in a cell described herein, the cell is deficient in the machinery necessary for homology directed repair (HDR). In some embodiments, the cell is a hematopoietic stem cell (HSC), such as a long-term engrafting hematopoietic stem cell (LT-HSC). In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell.

In some embodiments, according to any of the methods described herein employing a double-stranded donor nucleic acid, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is cleaved at the second and/or third recognition sequence following introduction into the cell. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the methods described herein employing a double-stranded donor nucleic acid, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the methods described herein employing a nuclease or nucleic acid encoding the nuclease, the nuclease is an RNA-guided endonuclease (RGEN). In some embodiments, the RGEN is a Cas9 nuclease. In some embodiments, the method further comprises introducing into the cell a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the method comprises introducing into the cell a ribonucleoprotein (RNP) comprising the RGEN and the gRNA. In some embodiments, the method comprises introducing into the cell a nucleic acid encoding the RGEN. In some embodiments, the nucleic acid encoding the RGEN is an mRNA or a plasmid.

In some embodiments, according to any of the methods described herein employing an RGEN or nucleic acid encoding the RGEN, each of the recognition sequences for the nuclease in the target locus and double-stranded donor nucleic acid is a protospacer sequence. In some embodiments, the method further comprises introducing into the cell one or more gRNAs targeting one or more of the protospacer sequences. In some embodiments, each of the protospacers in the target locus and double-stranded donor nucleic acid are the same, and the method comprises introducing into the cell one gRNA targeting the protospacer sequence.

In some embodiments, provided herein is a method for integrating an exogenous nucleic acid into a target locus in a cell, wherein the cell genome comprises a first protospacer in the target locus, comprising introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the DAP is shorter in length than the gRNA spacer by at least about 1 (such as at least about any of 2, 3, or more) nucleotide. In some embodiments, the DAP comprises at least about 1 (such as at least about any of 2, 3, or more) nucleotide mismatch with the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the cell is deficient in the machinery necessary for homology directed repair (HDR). In some embodiments, the cell is an HSC, such as an LT-HSC. In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell. In some embodiments, the method comprises introducing into the cell an RNP comprising the RGEN and the gRNA. In some embodiments, the method comprises introducing into the cell a nucleic acid encoding the RGEN. In some embodiments, the nucleic acid encoding the RGEN is an mRNA or a plasmid. In some embodiments, the RGEN is Cas9.

In some embodiments, provided herein is a method for integrating an exogenous nucleic acid into a target locus in a cell, wherein the cell genome comprises a first protospacer in the target locus, comprising introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, provided herein is a method for integrating an exogenous nucleic acid into a target locus in a cell, wherein the cell genome comprises a first protospacer in the target locus, comprising introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the methods of genome modification described herein, the nuclease or nucleic acid encoding the nuclease is introduced into the cell before the donor nucleic acid is introduced into the cell. In some embodiments, the nuclease or nucleic acid encoding the nuclease is introduced into the cell no more than 1 hour before the donor nucleic acid is introduced into the cell. In some embodiments, the nuclease or nucleic acid encoding the nuclease is introduced into the cell no more than 5 minutes before the donor nucleic acid is introduced into the cell. In some embodiments, introducing the nuclease or nucleic acid encoding the nuclease into the cell comprises introducing an RNP comprising an RGEN and a gRNA into the cell. In some embodiments, the donor nucleic acid is a double-stranded virus genome, e.g., an scAAV genome.

In some embodiments, according to any of the methods of genome modification described herein, the method comprises introducing into the cell an RNP comprising an RGEN and a gRNA before the donor nucleic acid is introduced into the cell. In some embodiments, the RNP is introduced into the cell no more than 1 hour before the donor nucleic acid is introduced into the cell. In some embodiments, the RNP is introduced into the cell no more than 5 minutes before the donor nucleic acid is introduced into the cell. In some embodiments, the donor nucleic acid is a double-stranded virus genome, e.g., an scAAV genome.

In some embodiments, according to any of the methods of genome modification described herein, the cell is cultured under hypoxic conditions.

In some embodiments, according to any of the methods of genome modification described herein, the cell is cultured no longer than about 48 hours prior to introducing the nuclease or nucleic acid encoding the nuclease and the donor nucleic acid into the cell. In some embodiments, the cell is cultured no longer than about 24 hours prior to introducing the nuclease or nucleic acid encoding the nuclease and the donor nucleic acid into the cell. In some embodiments, the cell is cultured no longer than about 2 hours prior to introducing the nuclease or nucleic acid encoding the nuclease and the donor nucleic acid into the cell. In some embodiments, introducing the nuclease or nucleic acid encoding the nuclease into the cell comprises introducing an RNP comprising an RGEN and a gRNA into the cell. In some embodiments, the donor nucleic acid is a double-stranded virus genome, e.g., an scAAV genome.

In some embodiments, according to any of the methods of genome modification described herein, the cell is cultured in the presence of a Notch ligand. In some embodiments, the Notch ligand is a Delta-like Notch ligand (DLL), Jagged-1, Jagged-2, or a conjugate thereof. In some embodiments, the Delta-like Notch ligand is DLL1, DLL3, or DLL4. In some embodiments, the Notch ligand is Fc-DLL1, Fc-DLL3, Fc-DLL4, Fc-Jagged-1, or Fc-Jagged-2.

In one aspect, provided herein is a method for genome modification at a target locus in an HSC, comprising: (a) introducing a nuclease or nucleic acid encoding the nuclease into the HSC, wherein the target locus comprises a first recognition sequence for the nuclease; (b) introducing a double-stranded donor nucleic acid into the HSC, wherein the double-stranded nucleic acid comprises an exogenous nucleic acid sequence and is configured to be inserted into the target locus by NHEJ. In some embodiments, the HSC is a long-term engrafting HSC (LT-HSC) or a SCID-repopulating cell. In some embodiments, the HSC is characterized by one or more (such as any of 2, 3, 4, or 5) of the following markers: Lin⁻/CD34⁻/CD38⁻/CD90⁻/CD45RA⁻. In some embodiments, Lin⁻ is characterized as one or more (such as any of 2, 3, 4, 5, 6, 7, or 8) of CD235a, CD41a, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻.

In some embodiments, provided herein is a method for integrating an exogenous nucleic acid into a target locus in an HSC, wherein the HSC genome comprises a first protospacer in the target locus, comprising introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the method comprises introducing into the HSC an RNP comprising the RGEN and the gRNA. In some embodiments, the method comprises introducing into the HSC a nucleic acid encoding the RGEN. In some embodiments, the nucleic acid encoding the RGEN is an mRNA or a plasmid. In some embodiments, the RGEN is Cas9.

In some embodiments, provided herein is a method for integrating an exogenous nucleic acid into a target locus in an HSC, wherein the HSC genome comprises a first protospacer in the target locus, comprising introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, provided herein is a method for integrating an exogenous nucleic acid into a target locus in an HSC, wherein the HSC genome comprises a first protospacer in the target locus, comprising introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In one aspect, provided herein is a method for genome modification at a target locus in a quiescent T cell, comprising: (a) introducing a nuclease or nucleic acid encoding the nuclease into the quiescent T cell, wherein the target locus comprises a first recognition sequence for the nuclease; (b) introducing a double-stranded donor nucleic acid into the quiescent T cell, wherein the double-stranded nucleic acid comprises an exogenous nucleic acid sequence and is configured to be inserted into the target locus by NHEJ. In some embodiments, the quiescent T cell is a non-activated T cell.

In some embodiments, provided herein is a method for integrating an exogenous nucleic acid into a target locus in a quiescent T cell, wherein the quiescent T cell genome comprises a first protospacer in the target locus, comprising introducing into the quiescent T cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the method comprises introducing into the quiescent T cell an RNP comprising the RGEN and the gRNA. In some embodiments, the method comprises introducing into the quiescent T cell a nucleic acid encoding the RGEN. In some embodiments, the nucleic acid encoding the RGEN is an mRNA or a plasmid. In some embodiments, the RGEN is Cas9.

In some embodiments, provided herein is a method for integrating an exogenous nucleic acid into a target locus in a quiescent T cell, wherein the quiescent T cell genome comprises a first protospacer in the target locus, comprising introducing into the quiescent T cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, provided herein is a method for integrating an exogenous nucleic acid into a target locus in a quiescent T cell, wherein the quiescent T cell genome comprises a first protospacer in the target locus, comprising introducing into the quiescent T cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In one aspect, provided herein is a method for genome modification at a target locus in an HDR-deficient cell, comprising: (a) introducing a nuclease or nucleic acid encoding the nuclease into the HDR-deficient cell, wherein the target locus comprises a first recognition sequence for the nuclease; (b) introducing a double-stranded donor nucleic acid into the HDR-deficient cell, wherein the double-stranded nucleic acid comprises an exogenous nucleic acid sequence and is configured to be inserted into the target locus by NHEJ. In some embodiments, the method further comprises culturing the HDR-deficient cell for a time sufficient for integration of the double-stranded donor nucleic acid into the target locus, wherein the steps are carried out such that the insertion efficiency for the double-stranded donor nucleic acid is at least about 4% (such as at least about any of 5%, 6%, 7%, 8%, 9%, 10%, or more). In some embodiments, the HDR-deficient cell is any cell type that is not dividing, not replicating, and thus, does not express the machinery for HDR.

In some embodiments, provided herein is a method for integrating an exogenous nucleic acid into a target locus in an HDR-deficient cell, wherein the HDR-deficient cell genome comprises a first protospacer in the target locus, comprising introducing into the HDR-deficient cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the method comprises introducing into the HDR-deficient cell an RNP comprising the RGEN and the gRNA. In some embodiments, the method comprises introducing into the HDR-deficient cell a nucleic acid encoding the RGEN. In some embodiments, the nucleic acid encoding the RGEN is an mRNA or a plasmid. In some embodiments, the RGEN is Cas9.

In some embodiments, provided herein is a method for integrating an exogenous nucleic acid into a target locus in an HDR-deficient cell, wherein the HDR-deficient cell genome comprises a first protospacer in the target locus, comprising introducing into the HDR-deficient cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, provided herein is a method for integrating an exogenous nucleic acid into a target locus in an HDR-deficient cell, wherein the HDR-deficient cell genome comprises a first protospacer in the target locus, comprising introducing into the HDR-deficient cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

### Engineered Cells

In some aspects, provided herein is an engineered cell, such as an engineered mammalian cell (e.g., HSC, T cell), comprising an exogenous nucleic acid sequence integrated at a target chromosomal locus, wherein the exogenous nucleic acid sequence has been integrated at the target locus by NHEJ. In some embodiments, the engineered cell is produced by any of the methods of genome editing described herein. The term “engineered cell” refers not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

In some embodiments, provided herein is an engineered cell (e.g., an HSC, such as an LT-HSC, or a T cell, such as a quiescent T cell) comprising: (a) a target locus comprising a first recognition sequence for a nuclease; (b) the nuclease or a nucleic acid encoding the nuclease; and (c) a double-stranded donor nucleic acid comprising an exogenous nucleic acid sequence, wherein the double-stranded donor nucleic acid is configured to be inserted into the target locus by a homology-independent mechanism (e.g., NHEJ).

In some embodiments, provided herein is an engineered cell (e.g., an HSC, such as an LT-HSC, or a T cell, such as a quiescent T cell) prepared by a method for genome modification at a target locus in a cell according to any of the embodiments described herein. In some embodiments, the method comprises: (a) introducing a nuclease or nucleic acid encoding the nuclease into an input cell, wherein the target locus comprises a first recognition sequence for the nuclease; and (b) introducing a double-stranded donor nucleic acid into the input cell, wherein the double-stranded nucleic acid comprises an exogenous nucleic acid sequence and is configured to be inserted into the target locus by a homology-independent mechanism (e.g., NHEJ).

In some embodiments, according to any of the engineered cells described herein, the cell is deficient in the machinery necessary for homology directed repair (HDR). In some embodiments, the cell is a hematopoietic stem cell (HSC), such as a long-term engrafting hematopoietic stem cell (LT-HSC). In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell.

In some embodiments, according to any of the engineered cells described herein comprising a double-stranded donor nucleic acid, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the engineered cells described herein comprising a double-stranded donor nucleic acid, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the engineered cells described herein comprising a nuclease or nucleic acid encoding the nuclease, the nuclease is an RNA-guided endonuclease (RGEN). In some embodiments, the RGEN is a Cas9 nuclease. In some embodiments, the engineered cells further comprise a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the engineered cells comprises a ribonucleoprotein (RNP) comprising the RGEN and the gRNA. In some embodiments, the engineered cells comprises a nucleic acid encoding the RGEN. In some embodiments, the nucleic acid encoding the RGEN is an mRNA or a plasmid.

In some embodiments, according to any of the engineered cells described herein comprising an RGEN or nucleic acid encoding the RGEN, each of the recognition sequences for the nuclease in the target locus and double-stranded donor nucleic acid is a protospacer sequence. In some embodiments, the engineered cells further comprise one or more gRNAs targeting one or more of the protospacer sequences. In some embodiments, each of the protospacers in the target locus and double-stranded donor nucleic acid are the same, and the engineered cells comprise one gRNA targeting the protospacer sequence.

The engineered cells described herein in some embodiments comprise i) a first nucleic acid comprising a first protospacer and ii) a gRNA comprising a spacer, wherein the first protospacer is a DAP having an incomplete match to the spacer, and wherein the degree to which the first protospacer matches the spacer is sufficient to allow for modification of the first nucleic acid at the first protospacer. In some embodiments, the DAP is shorter in length than the spacer by at least about 1 (such as at least about any of 2, 3, or more) nucleotide. In some embodiments, the DAP comprises at least about 1 (such as at least about any of 2, 3, or more) nucleotide mismatch with the spacer. In some embodiments, the engineered cells further comprises a second nucleic acid comprising a second protospacer, wherein the second protospacer is a complete match to the spacer.

In some embodiments, according to any of the engineered cells described herein, the engineered cells comprise a) an RGEN or nucleic acid encoding the RGEN, b) a gRNA comprising a spacer or nucleic acid encoding the gRNA, wherein the gRNA is capable of targeting the RGEN to cleave a first protospacer in a target locus in the engineered cells into which an exogenous nucleic acid is to be inserted, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the engineered cells are deficient in the machinery necessary for homology directed repair (HDR). In some embodiments, the engineered cells are HSCs, such as LT-HSCs. In some embodiments, the engineered cells are SCID-repopulating cells. In some embodiments, the engineered cells are characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the engineered cells are quiescent T cells.

In some embodiments, according to any of the engineered cells described herein, the engineered cells comprise a) an RGEN or nucleic acid encoding the RGEN, b) a gRNA comprising a spacer or nucleic acid encoding the gRNA, wherein the gRNA is capable of targeting the RGEN to cleave a first protospacer in a target locus in the engineered cells into which an exogenous nucleic acid is to be inserted, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the engineered cells described herein, the engineered cells comprise a) an RGEN or nucleic acid encoding the RGEN, b) a gRNA comprising a spacer or nucleic acid encoding the gRNA, wherein the gRNA is capable of targeting the RGEN to cleave a first protospacer in a target locus in the engineered cells into which an exogenous nucleic acid is to be inserted, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, the engineered cells are HSCs, including both LT-HSCs and short-term engrafting HSCs (ST-HSCs), such as HSCs that are CD34⁺ but CD90^{+/-}, CD38^{+/-}, and/or CD45RA^{+/-}.

The HSCs can be collected in accordance with known techniques and enriched or depleted by known techniques such as affinity binding to antibodies such as flow cytometry and/or immunomagnetic selection. In some embodiments, the HSCs are autologous HSCs obtained from a patient.

In some embodiments, the engineered cells are T cells, or precursor cells capable of differentiating into T cells. In some embodiments, the engineered cells are CD3⁺, CD8⁺, and/or CD4⁺ T lymphocytes. In some embodiments, the engineered cells are CD8⁺ T cytotoxic lymphocyte cells, which may include naïve CD8⁺ T cells, central memory CD8⁺ T cells, effector memory CD8⁺ T cells, or bulk CD8⁺ T cells.

The lymphocytes (T lymphocytes) can be collected in accordance with known techniques and enriched or depleted by known techniques such as affinity binding to antibodies such as flow cytometry and/or immunomagnetic selection. In some embodiments, the T cells are autologous T cells obtained from a patient.

The present disclosure further provides, in some embodiments, a composition comprising an engineered cell as described herein.

### Methods of Engineered Cell Engraftment

In some embodiments, provided herein is a method of engrafting an edited HSC comprising an exogenous nucleic acid sequence inserted at a target locus in an individual, comprising administering a population of edited HSCs according to any of the embodiments described herein to the individual. In some embodiments, the edited HSCs persist in the individual for at least about any of 1, 2, 3, 4, 5, 6, 7, 8, 9, 10, 11, 12, 13, 14, 15, 16, or more weeks. In some embodiments, the population of edited HSCs are prepared according to any of the methods described herein. In some embodiments, the amount of engraftment of edited HSC in the individual is the same or greater than the amount of engraftment of corresponding edited HSCs prepared using a homology-dependent mechanism. In some embodiments, the population of edited HSCs is contained in an output population of HSCs derived from an input population of HSCs obtained from the individual. In some embodiments, the input population of HSCs obtained from the individual comprises a mixed population of HSCs comprising LT-HSCs and ST-HSCs, and the population of edited HSCs that engraft comprise edited LT-HSCs. In some embodiments, the method comprises administering the output population of HSCs to the individual.

In some embodiments, according to any of the methods of engrafting an edited HSC comprising an exogenous nucleic acid sequence inserted at a target locus in an individual described herein, the edited HSC is prepared by a method comprising: (a) introducing a nuclease or nucleic acid encoding the nuclease into the HSC, wherein the target locus comprises a first recognition sequence for the nuclease; and (b) introducing a double-stranded donor nucleic acid into the HSC, wherein the double-stranded nucleic acid comprises an exogenous nucleic acid sequence and is configured to be inserted into the target locus by NHEJ. In some embodiments, the HSC is a long-term engrafting hematopoietic stem cell (LT-HSC). In some embodiments, the HSC is a SCID-repopulating cell. In some embodiments, the HSC is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a, CD41a, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻.

In some embodiments, according to any of the methods of engrafting an edited HSC comprising an exogenous nucleic acid sequence inserted at a target locus in an individual described herein, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is cleaved at the second and/or third recognition sequence following introduction into the cell. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the methods of engrafting an edited HSC comprising an exogenous nucleic acid sequence inserted at a target locus in an individual described herein, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the methods of engrafting an edited HSC comprising an exogenous nucleic acid sequence inserted at a target locus in an individual described herein, the nuclease is an RNA-guided endonuclease (RGEN). In some embodiments, the RGEN is a Cas9 nuclease. In some embodiments, the method of preparing the edited HSC further comprises introducing into the HSC a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the method of preparing the edited HSC comprises introducing into the HSC a ribonucleoprotein (RNP) comprising the RGEN and the gRNA. In some embodiments, the method of preparing the edited HSC comprises introducing into the HSC a nucleic acid encoding the RGEN. In some embodiments, the nucleic acid encoding the RGEN is an mRNA or a plasmid.

In some embodiments, according to any of the methods of engrafting an edited HSC comprising an exogenous nucleic acid sequence inserted at a target locus in an individual described herein, each of the recognition sequences for the nuclease in the target locus and double-stranded donor nucleic acid is a protospacer sequence. In some embodiments, the method of preparing the edited HSC further comprises introducing into the HSC one or more gRNAs targeting one or more of the protospacer sequences. In some embodiments, each of the protospacers in the target locus and double-stranded donor nucleic acid are the same, and the method comprises introducing into the cell one gRNA targeting the protospacer sequence.

In some embodiments, according to any of the methods of preparing an edited HSC comprising an exogenous nucleic acid sequence inserted at a target locus employed in a method of engrafting the edited HSC in an individual described herein, the HSC genome comprises a first protospacer in the target locus, and the method comprises introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the DAP is shorter in length than the gRNA spacer by at least about 1 (such as at least about any of 2, 3, or more) nucleotide. In some embodiments, the DAP comprises at least about 1 (such as at least about any of 2, 3, or more) nucleotide mismatch with the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the method comprises introducing into the HSC an RNP comprising the RGEN and the gRNA. In some embodiments, the method comprises introducing into the HSC a nucleic acid encoding the RGEN. In some embodiments, the nucleic acid encoding the RGEN is an mRNA or a plasmid. In some embodiments, the RGEN is Cas9.

In some embodiments, according to any of the methods of preparing an edited HSC comprising an exogenous nucleic acid sequence inserted at a target locus employed in a method of engrafting the edited HSC in an individual described herein, the HSC genome comprises a first protospacer in the target locus, and the method comprises introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the methods of preparing an edited HSC comprising an exogenous nucleic acid sequence inserted at a target locus employed in a method of engrafting the edited HSC in an individual described herein, the HSC genome comprises a first protospacer in the target locus, and the method comprises introducing into the HSC a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the methods of preparing an edited HSC comprising an exogenous nucleic acid sequence inserted at a target locus employed in a method of engrafting the edited HSC in an individual described herein, the nuclease or nucleic acid encoding the nuclease is introduced into the HSC before the donor nucleic acid is introduced into the HSC. In some embodiments, the nuclease or nucleic acid encoding the nuclease is introduced into the HSC no more than 1 hour before the donor nucleic acid is introduced into the HSC. In some embodiments, the nuclease or nucleic acid encoding the nuclease is introduced into the HSC no more than 5 minutes before the donor nucleic acid is introduced into the HSC. In some embodiments, introducing the nuclease or nucleic acid encoding the nuclease into the HSC comprises introducing an RNP comprising an RGEN and a gRNA into the HSC. In some embodiments, the donor nucleic acid is a double-stranded virus genome, e.g., an scAAV genome.

In some embodiments, according to any of the methods of preparing an edited HSC comprising an exogenous nucleic acid sequence inserted at a target locus employed in a method of engrafting the edited HSC in an individual described herein, the method comprises introducing into the HSC an RNP comprising an RGEN and a gRNA before the donor nucleic acid is introduced into the HSC. In some embodiments, the RNP is introduced into the HSC no more than 1 hour before the donor nucleic acid is introduced into the HSC. In some embodiments, the RNP is introduced into the HSC no more than 5 minutes before the donor nucleic acid is introduced into the HSC. In some embodiments, the donor nucleic acid is a double-stranded virus genome, e.g., an scAAV genome.

In some embodiments, according to any of the methods of preparing an edited HSC comprising an exogenous nucleic acid sequence inserted at a target locus employed in a method of engrafting the edited HSC in an individual described herein, the HSC is cultured under hypoxic conditions.

In some embodiments, according to any of the methods of preparing an edited HSC comprising an exogenous nucleic acid sequence inserted at a target locus employed in a method of engrafting the edited HSC in an individual described herein, the HSC is cultured no longer than about 48 hours prior to introducing the nuclease or nucleic acid encoding the nuclease and the donor nucleic acid into the HSC. In some embodiments, the HSC is cultured no longer than about 24 hours prior to introducing the nuclease or nucleic acid encoding the nuclease and the donor nucleic acid into the HSC. In some embodiments, the HSC is cultured no longer than about 2 hours prior to introducing the nuclease or nucleic acid encoding the nuclease and the donor nucleic acid into the HSC. In some embodiments, introducing the nuclease or nucleic acid encoding the nuclease into the HSC comprises introducing an RNP comprising an RGEN and a gRNA into the HSC. In some embodiments, the donor nucleic acid is a double-stranded virus genome, e.g., an scAAV genome.

In some embodiments, according to any of the methods of preparing an edited HSC comprising an exogenous nucleic acid sequence inserted at a target locus employed in a method of engrafting the edited HSC in an individual described herein, the HSC is cultured in the presence of a Notch ligand. In some embodiments, the Notch ligand is a Delta-like Notch ligand (DLL), Jagged-1, Jagged-2, or a conjugate thereof. In some embodiments, the Delta-like Notch ligand is DLL1, DLL3, or DLL4. In some embodiments, the Notch ligand is Fc-DLL1, Fc-DLL3, Fc-DLL4, Fc-Jagged-1, or Fc-Jagged-2.

### Methods of Treatment

In some embodiments, provided herein is a method of treating a disease or condition in a subject in need thereof, wherein the disease or condition is characterized by deficient expression of a functional protein, comprising administering to the subject a cell edited according to any of the methods described herein to express a functional form of the protein.

In some embodiments, the disease is Severe Combined Immunodeficiency (SCID), and the method comprises administering to the subject a population of HSCs edited according to any of the methods described herein to express a functional form a protein mutated in the subject. In some embodiments, the HSCs are edited to express a protein involved in lymphoid development, e.g., IL2Rg, RAG1, or IL7R. In some embodiments, the HSCs are edited to express a protein involved in lymphocyte proliferation and/or metabolism, e.g., ADA or PNP. In some embodiments, the disease is Gaucher disease, Fabry disease, mucopolysaccharidosis types I-IX, or adrenoleukodystrophy.

In some embodiments, according to any of the methods of treating a disease or condition in a subject in need thereof, the edited cell is prepared by a method comprising: (a) introducing a nuclease or nucleic acid encoding the nuclease into the cell, wherein the target locus comprises a first recognition sequence for the nuclease; and (b) introducing a double-stranded donor nucleic acid into the cell, wherein the double-stranded nucleic acid comprises an exogenous nucleic acid sequence and is configured to be inserted into the target locus by NHEJ. In some embodiments, the cell is deficient in the machinery necessary for homology directed repair (HDR). In some embodiments, the cell is a hematopoietic stem cell (HSC), such as a long-term engrafting hematopoietic stem cell (LT-HSC). In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell.

In some embodiments, according to any of the methods of treating a disease or condition in a subject in need thereof, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is cleaved at the second and/or third recognition sequence following introduction into the cell. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the methods of treating a disease or condition in a subject in need thereof, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the methods of treating a disease or condition in a subject in need thereof, the nuclease is an RNA-guided endonuclease (RGEN). In some embodiments, the RGEN is a Cas9 nuclease. In some embodiments, the method of preparing the edited cell further comprises introducing into the cell a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the method of preparing the edited cell comprises introducing into the cell a ribonucleoprotein (RNP) comprising the RGEN and the gRNA. In some embodiments, the method of preparing the edited cell comprises introducing into the cell a nucleic acid encoding the RGEN. In some embodiments, the nucleic acid encoding the RGEN is an mRNA or a plasmid.

In some embodiments, according to any of the methods of treating a disease or condition in a subject in need thereof, each of the recognition sequences for the nuclease in the target locus and double-stranded donor nucleic acid is a protospacer sequence. In some embodiments, the method of preparing the edited cell further comprises introducing into the cell one or more gRNAs targeting one or more of the protospacer sequences. In some embodiments, each of the protospacers in the target locus and double-stranded donor nucleic acid are the same, and the method comprises introducing into the cell one gRNA targeting the protospacer sequence.

In some embodiments, according to any of the methods of preparing an edited cell comprising an exogenous nucleic acid sequence inserted at a target locus employed in a method of treating a disease or condition in a subject in need thereof described herein, the cell genome comprises a first protospacer in the target locus, and the method comprises introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the DAP is shorter in length than the gRNA spacer by at least about 1 (such as at least about any of 2, 3, or more) nucleotide. In some embodiments, the DAP comprises at least about 1 (such as at least about any of 2, 3, or more) nucleotide mismatch with the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the method comprises introducing into the cell an RNP comprising the RGEN and the gRNA. In some embodiments, the method comprises introducing into the cell a nucleic acid encoding the RGEN. In some embodiments, the nucleic acid encoding the RGEN is an mRNA or a plasmid. In some embodiments, the RGEN is Cas9.

In some embodiments, according to any of the methods of preparing an edited cell comprising an exogenous nucleic acid sequence inserted at a target locus employed in a method of treating a disease or condition in a subject in need thereof described herein, the cell genome comprises a first protospacer in the target locus, and the method comprises introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the methods of preparing an edited cell comprising an exogenous nucleic acid sequence inserted at a target locus employed in a method of treating a disease or condition in a subject in need thereof described herein, the cell genome comprises a first protospacer in the target locus, and the method comprises introducing into the cell a) an RGEN or nucleic acid encoding the RGEN, b) a gRNA comprising a spacer or nucleic acid encoding the gRNA, wherein the gRNA is capable of targeting the RGEN to cleave the first protospacer in the target locus, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation.

In some embodiments, according to any of the methods of preparing an edited cell comprising an exogenous nucleic acid sequence inserted at a target locus employed in a method of treating a disease or condition in a subject in need thereof described herein, the nuclease or nucleic acid encoding the nuclease is introduced into the cell before the donor nucleic acid is introduced into the cell. In some embodiments, the nuclease or nucleic acid encoding the nuclease is introduced into the cell no more than 1 hour before the donor nucleic acid is introduced into the cell. In some embodiments, the nuclease or nucleic acid encoding the nuclease is introduced into the cell no more than 5 minutes before the donor nucleic acid is introduced into the cell. In some embodiments, introducing the nuclease or nucleic acid encoding the nuclease into the cell comprises introducing an RNP comprising an RGEN and a gRNA into the cell. In some embodiments, the donor nucleic acid is a double-stranded virus genome, e.g., an scAAV genome.

In some embodiments, according to any of the methods of preparing an edited cell comprising an exogenous nucleic acid sequence inserted at a target locus employed in a method of treating a disease or condition in a subject in need thereof described herein, the method comprises introducing into the cell an RNP comprising an RGEN and a gRNA before the donor nucleic acid is introduced into the cell. In some embodiments, the RNP is introduced into the cell no more than 1 hour before the donor nucleic acid is introduced into the cell. In some embodiments, the RNP is introduced into the cell no more than 5 minutes before the donor nucleic acid is introduced into the cell. In some embodiments, the donor nucleic acid is a double-stranded virus genome, e.g., an scAAV genome.

In some embodiments, according to any of the methods of preparing an edited cell comprising an exogenous nucleic acid sequence inserted at a target locus employed in a method of treating a disease or condition in a subject in need thereof described herein, the cell is cultured under hypoxic conditions.

In some embodiments, according to any of the methods of preparing an edited cell comprising an exogenous nucleic acid sequence inserted at a target locus employed in a method of treating a disease or condition in a subject in need thereof described herein, the cell is cultured no longer than about 48 hours prior to introducing the nuclease or nucleic acid encoding the nuclease and the donor nucleic acid into the cell. In some embodiments, the cell is cultured no longer than about 24 hours prior to introducing the nuclease or nucleic acid encoding the nuclease and the donor nucleic acid into the cell. In some embodiments, the cell is cultured no longer than about 2 hours prior to introducing the nuclease or nucleic acid encoding the nuclease and the donor nucleic acid into the cell. In some embodiments, introducing the nuclease or nucleic acid encoding the nuclease into the cell comprises introducing an RNP comprising an RGEN and a gRNA into the cell. In some embodiments, the donor nucleic acid is a double-stranded virus genome, e.g., an scAAV genome.

In some embodiments, according to any of the methods of preparing an edited cell comprising an exogenous nucleic acid sequence inserted at a target locus employed in a method of treating a disease or condition in a subject in need thereof described herein, the cell is cultured in the presence of a Notch ligand. In some embodiments, the Notch ligand is a Delta-like Notch ligand (DLL), Jagged-1, Jagged-2, or a conjugate thereof. In some embodiments, the Delta-like Notch ligand is DLL1, DLL3, or DLL4. In some embodiments, the Notch ligand is Fc-DLL1, Fc-DLL3, Fc-DLL4, Fc-Jagged-1, or Fc-Jagged-2.

### Compositions

Provided herein are compositions that comprise one or more elements of a system for generating engineered cells as set forth in this disclosure.

In some embodiments, provided herein is a composition comprising one or more of a) a nuclease or nucleic acid encoding the nuclease, wherein the nuclease is capable of mediating genome editing at a target locus comprising a first recognition sequence for the nuclease; and b) a double-stranded donor nucleic acid, wherein the double-stranded donor nucleic acid comprises an exogenous nucleic acid sequence and is configured to be inserted into the target locus by NHEJ. In some embodiments, the composition further comprises a cell to be edited.

In some embodiments, according to any of the compositions described herein comprising a double-stranded donor nucleic acid, the double-stranded donor nucleic acid further comprises a second recognition sequence for the nuclease flanking a first end of the exogenous nucleic acid sequence. In some embodiments, the double-stranded donor nucleic acid further comprises a third recognition sequence flanking a second end of the exogenous nucleic acid sequence. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

In some embodiments, according to any of the compositions described herein comprising a double-stranded donor nucleic acid, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

In some embodiments, according to any of the compositions described herein comprising a nuclease or nucleic acid encoding the nuclease, the nuclease is an RNA-guided endonuclease (RGEN). In some embodiments, the RGEN is a Cas9 nuclease. In some embodiments, the composition further comprises a gRNA capable of guiding the RGEN to cleave the first recognition sequence in the target locus. In some embodiments, the gRNA is capable of guiding the RGEN to cleave one or more recognition sequences in the donor nucleic acid. In some embodiments, the composition comprises a ribonucleoprotein (RNP) comprising the RGEN and the gRNA. In some embodiments, the composition comprises a nucleic acid encoding the RGEN. In some embodiments, the nucleic acid encoding the RGEN is an mRNA or a plasmid.

In some embodiments, according to any of the compositions described herein comprising an RGEN or nucleic acid encoding the RGEN, each of the recognition sequences for the nuclease in the target locus and double-stranded donor nucleic acid is a protospacer sequence. In some embodiments, the composition further comprises one or more gRNAs targeting one or more of the protospacer sequences. In some embodiments, each of the protospacers in the target locus and double-stranded donor nucleic acid are the same, and the composition comprises one gRNA targeting the protospacer sequence.

The compositions described herein in some embodiments comprise one or more of i) a first nucleic acid comprising a first protospacer and ii) a gRNA comprising a spacer, wherein the first protospacer is a DAP having an incomplete match to the spacer, and wherein the degree to which the first protospacer matches the spacer is sufficient to allow for modification of the first nucleic acid at the first protospacer. In some embodiments, the DAP is shorter in length than the spacer by at least about 1 (such as at least about any of 2, 3, or more) nucleotide. In some embodiments, the DAP comprises at least about 1 (such as at least about any of 2, 3, or more) nucleotide mismatch with the spacer. In some embodiments, the composition further comprises a second nucleic acid comprising a second protospacer, wherein the second protospacer is a complete match to the spacer.

In some embodiments, according to any of the compositions described herein, the composition comprises one or more of a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and c) a double-stranded donor nucleic acid, wherein the donor nucleic acid comprises the exogenous nucleic acid flanked on one or both sides by a second protospacer, wherein the gRNA is capable of targeting the RGEN to cleave the second protospacer in the donor nucleic acid, and wherein the donor nucleic acid is configured such that the exogenous nucleic acid is capable of being inserted into the target locus by NHEJ. In some embodiments, the first protospacer and the second protospacer completely match the gRNA spacer. In some embodiments, the first protospacer in the target locus completely matches the gRNA spacer, and the second protospacer in the donor nucleic acid is a DAP that does not completely match the gRNA spacer. In some embodiments, the first protospacer in the target locus is a DAP that does not completely match the gRNA spacer, and the second protospacer in the donor nucleic acid completely matches the gRNA spacer. In some embodiments, the donor nucleic acid comprises the exogenous nucleic acid flanked on both sides by the second protospacer. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. For example, in some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the composition further comprises a cell comprising the target locus. In some embodiments, the cell is deficient in the machinery necessary for homology directed repair (HDR). In some embodiments, the cell is a hematopoietic stem cell (HSC), such as a long-term engrafting hematopoietic stem cell (LT-HSC). In some embodiments, the cell is a SCID-repopulating cell. In some embodiments, the cell is characterized by one or more (such as any of 2, 3, 4, or 5) of the following markers: Lin⁻/CD34⁺/CD38⁻/CD90⁺/CD45RA⁻. In some embodiments, Lin⁻ is characterized as one or more (such as any of 2, 3, 4, 5, 6, 7, or 8) of CD235a⁻, CD41a⁻, CD3⁻, CD19⁻, CD14⁻, CD16⁻, CD20⁻, and CD56⁻. In some embodiments, Lin⁻ is characterized as CD235a⁻/CD41a⁻/CD3⁻/CD19⁻/CD14⁻/CD16⁻/CD20⁻/CD56⁻. In some embodiments, the cell is a quiescent T cell.

In some embodiments, according to any of the compositions described herein, the composition comprises one or more of a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and wherein the first protospacer completely matches the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer is a DAP that does not completely match the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the composition further comprises a cell comprising the target locus.

In some embodiments, according to any of the compositions described herein, the composition comprises one or more of a) an RGEN or nucleic acid encoding the RGEN, b) a gRNA comprising a spacer or nucleic acid encoding the gRNA, wherein the gRNA is capable of targeting the RGEN to cleave a first protospacer in a target locus into which an exogenous nucleic acid is to be inserted, and wherein the first protospacer is a DAP that does not completely match the gRNA spacer, and c) a double-stranded donor nucleic acid, wherein the donor nucleic acid comprises the exogenous nucleic acid flanked on both sides by a second protospacer, wherein the gRNA is capable of targeting the RGEN to cleave the second protospacer in the donor nucleic acid, wherein the second protospacer completely matches the gRNA spacer, and wherein the donor nucleic acid is configured such that the exogenous nucleic acid is capable of being inserted into the target locus by NHEJ. In some embodiments, the donor nucleic acid is configured such that i) insertion of the cleaved donor nucleic acid into the cleaved target locus in a desired orientation does not create a protospacer flanking the exogenous nucleic acid capable of being cleaved by the RGEN guided by the gRNA, and ii) insertion of the cleaved donor nucleic acid into the cleaved target locus in the other orientation creates a protospacer flanking the exogenous nucleic acid capable of being cleaved by the RGEN guided by the gRNA. In some embodiments, the first protospacer in the target locus is in a forward orientation, the exogenous nucleic acid in the donor nucleic acid is in a forward orientation, and the second protospacers in the donor nucleic acid are in a reverse orientation. In some embodiments, the composition further comprises a cell comprising the target locus.

Also provided herein are compositions that comprise a genetically modified cell, such as a mammalian cell, prepared as set forth in this disclosure.

Also provided herein are kits and systems including engineered cells and/or system elements for generating engineered cells provided and described herein. Thus, for example, provided herein is a kit comprising one or more of: a protein sequence as described herein; an expression vector as described herein; and/or a cell as described herein.

### Nucleic Acids

### Genome-Targeting Nucleic Acid or Guide RNA

The present disclosure provides a genome-targeting nucleic acid that can direct the activities of an associated polypeptide (e.g., a site-directed polypeptide or DNA endonuclease) to a specific target sequence within a target nucleic acid. In some embodiments, the genome-targeting nucleic acid is an RNA. A genome-targeting RNA is referred to as a “guide RNA” or “gRNA” herein. A guide RNA has at least a spacer sequence that hybridizes to a target nucleic acid sequence of interest and a CRISPR repeat sequence. In Type II systems, the gRNA also has a second RNA called the tracrRNA sequence. In the Type II guide RNA (gRNA), the CRISPR repeat sequence and tracrRNA sequence hybridize to each other to form a duplex. In the Type V guide RNA (gRNA), the crRNA forms a duplex. In both systems, the duplex binds a site-directed polypeptide such that the guide RNA and site-direct polypeptide form a complex. The genome-targeting nucleic acid provides target specificity to the complex by virtue of its association with the site-directed polypeptide. The genome-targeting nucleic acid thus directs the activity of the site-directed polypeptide.

In some embodiments, the genome-targeting nucleic acid is a double-molecule guide RNA. In some embodiments, the genome-targeting nucleic acid is a single-molecule guide RNA. A double-molecule guide RNA has two strands of RNA. The first strand has in the 5′ to 3′ direction, an optional spacer extension sequence, a spacer sequence and a minimum CRISPR repeat sequence. The second strand has a minimum tracrRNA sequence (complementary to the minimum CRISPR repeat sequence), a 3′ tracrRNA sequence and an optional tracrRNA extension sequence. A single-molecule guide RNA (sgRNA) in a Type II system has, in the 5′ to 3′ direction, an optional spacer extension sequence, a spacer sequence, a minimum CRISPR repeat sequence, a single-molecule guide linker, a minimum tracrRNA sequence, a 3′ tracrRNA sequence and an optional tracrRNA extension sequence. The optional tracrRNA extension may have elements that contribute additional functionality (e.g., stability) to the guide RNA. The single-molecule guide linker links the minimum CRISPR repeat and the minimum tracrRNA sequence to form a hairpin structure. The optional tracrRNA extension has one or more hairpins. A single-molecule guide RNA (sgRNA) in a Type V system has, in the 5′ to 3′ direction, a minimum CRISPR repeat sequence and a spacer sequence.

Exemplary genome-targeting nucleic acids are described in WO2018002719.

### Donor DNA or Donor Template

Site-directed polypeptides, such as a DNA endonuclease, can introduce double-strand breaks or single-strand breaks in nucleic acids, e.g., genomic DNA. The double-strand break can stimulate a cell's endogenous DNA-repair pathways (e.g., homology-dependent repair (HDR) or non-homologous end joining (NHEJ) or alternative non-homologous end joining (A-NHEJ) or microhomology-mediated end joining (MMEJ). NHEJ can repair cleaved target nucleic acid without the need for a homologous template. This can sometimes result in small deletions or insertions (indels) in the target nucleic acid at the site of cleavage, and can lead to disruption or alteration of gene expression.

When an exogenous DNA molecule is supplied in sufficient concentration inside the nucleus of a cell in which the double strand break occurs, the exogenous DNA can be inserted at the double strand break during the NHEJ repair process and thus become a permanent addition to the genome. Inclusion of nuclease target sites in the exogenous DNA greatly stimulates their insertion into the target site during NHEJ-mediated DNA repair. These exogenous DNA molecules are referred to as donor templates in some embodiments. If the donor template contains a coding sequence for a transgene optionally together with relevant regulatory sequences such as promoters, enhancers, polyA sequences and/or splice acceptor sequences (also referred to herein as a “donor cassette”), the transgene can be expressed from the integrated nucleic acid in the genome resulting in permanent expression for the life of the cell. Moreover, the integrated nucleic acid of the donor DNA template can be transmitted to the daughter cells when the cell divides.

In some embodiments, the donor DNA can be supplied with the nuclease or independently by a variety of different methods, for example by transfection, nano-particle, micro-injection, or viral transduction.

In some embodiments, according to any of the donor templates described herein comprising an exogenous nucleic acid sequence, the exogenous nucleic acid sequence is flanked on one or both sides by a gRNA target site. For example, such a donor template may comprise an exogenous nucleic acid sequence with a gRNA target site 5′ of the exogenous nucleic acid sequence and/or a gRNA target site 3′ of the exogenous nucleic acid sequence. In some embodiments, the donor template comprises an exogenous nucleic acid sequence with a gRNA target site 5′ of the exogenous nucleic acid sequence. In some embodiments, the donor template comprises an exogenous nucleic acid sequence with a gRNA target site 3′ of the exogenous nucleic acid sequence. In some embodiments, the donor template comprises an exogenous nucleic acid sequence with a gRNA target site 5′ of the exogenous nucleic acid sequence and a gRNA target site 3′ of the exogenous nucleic acid sequence. In some embodiments, the donor template comprises an exogenous nucleic acid sequence with a gRNA target site 5′ of the exogenous nucleic acid sequence and a gRNA target site 3′ of the exogenous nucleic acid sequence, and the two gRNA target sites comprise the same sequence. In some embodiments, the donor template comprises at least one gRNA target site, and the at least one gRNA target site in the donor template comprises the same sequence as a gRNA target site in a target locus into which the exogenous nucleic acid sequence of the donor template is to be integrated. In some embodiments, the donor template comprises an exogenous nucleic acid sequence with a gRNA target site 5′ of the exogenous nucleic acid sequence and a gRNA target site 3′ of the exogenous nucleic acid sequence, and the two gRNA target sites in the donor template comprises the same sequence as a gRNA target site in a target locus into which the exogenous nucleic acid sequence of the donor template is to be integrated. In some embodiments, the gRNA target site in the target locus is in a forward orientation, the exogenous nucleic acid in the donor nucleic acid is in a forward orientation, and the gRNA target sites in the donor nucleic acid are in a reverse orientation.

In some embodiments, the donor template is a double-stranded donor nucleic acid. In some embodiments, the double-stranded donor nucleic acid comprises an exogenous nucleic acid sequence and is configured to be inserted into a target locus by NHEJ. In some embodiments, the double-stranded donor nucleic acid further comprises a first recognition sequence for a first nuclease flanking a first end of the exogenous nucleic acid sequence. In some embodiments, the double-stranded donor nucleic acid comprises a second recognition sequence for a second nuclease flanking a second end of the exogenous nucleic acid sequence. In some embodiments, the first and second nucleases are the same nuclease. In some embodiments, the first and second recognition sequences are the same recognition sequence. In some embodiments, the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus. In some embodiments, the first and second nucleases are an RGEN, and the first and second recognition sequences are protospacer sequences.

In some embodiments, a double-stranded donor nucleic acid described herein comprises an exogenous nucleic acid sequence flanked on one or both ends by a protospacer. In some embodiments, the exogenous nucleic acid sequence is flanked on its 5′ end by a protospacer. In some embodiments, the exogenous nucleic acid sequence is flanked on its 3′ end by a protospacer. In some embodiments, the exogenous nucleic acid sequence is flanked on its 5′ and 3′ ends by a protospacer. In some embodiments, a protospacer sequence in a target locus is in a forward orientation, the exogenous nucleic acid in the donor nucleic acid is in a forward orientation, and the protospacers in the donor nucleic acid are in a reverse orientation.

In some embodiments, a double-stranded donor nucleic acid described herein comprises an exogenous nucleic acid sequence flanked on one or both ends by a delayed-action protospacer (DAP) having an incomplete match to the spacer of a gRNA, wherein the degree to which the DAP matches the spacer is sufficient to allow for modification of the donor nucleic acid at the DAP by an RGEN guided by the gRNA. In some embodiments, the DAP is shorter in length than the spacer by at least about 1 (such as at least about any of 2, 3, or more) nucleotide. In some embodiments, the DAP comprises at least about 1 (such as at least about any of 2, 3, or more) nucleotide mismatch with the spacer. For example, the gRNA may comprise a spacer from the polynucleotide sequence of SEQ ID NO: 8, and the DAP may comprise a protospacer from the polynucleotide sequence of any one of SEQ ID NOs: 16-28.

In some embodiments, according to any of the methods described herein employing a double-stranded donor nucleic acid, the double-stranded donor nucleic acid is a double-stranded virus genome. In some embodiments, the double-stranded virus genome is an adenovirus genome, a lentivirus genome (e.g., a dsDNA intermediate of a lentivirus RNA genome), or an adeno-associated virus (AAV) genome. In some embodiments, the AAV genome is a self-complementary AAV (scAAV) genome. In some embodiments, the scAAV genome is an scAAV6 genome. In some embodiments, the lentivirus genome is an integrase-deficient lentivirus genome.

### Nucleic Acid Encoding a Site-Directed Polypeptide or DNA Endonuclease

In some embodiments, the methods of genome editing and compositions therefore can use a nucleic acid sequence encoding a site-directed polypeptide or DNA endonuclease. The nucleic acid sequence encoding the site-directed polypeptide can be DNA or RNA. If the nucleic acid sequence encoding the site-directed polypeptide is RNA, it can be covalently linked to a gRNA sequence or exist as a separate sequence. In some embodiments, a peptide sequence of the site-directed polypeptide or DNA endonuclease can be used instead of the nucleic acid sequence thereof.

### Vectors

In another aspect, the present disclosure provides a nucleic acid having a nucleotide sequence encoding a genome-targeting nucleic acid of the disclosure, a site-directed polypeptide of the disclosure, and/or any nucleic acid or proteinaceous molecule necessary to carry out the embodiments of the methods of the disclosure. In some embodiments, such a nucleic acid is a vector (e.g., a recombinant expression vector).

Expression vectors contemplated include, but are not limited to, viral vectors based on vaccinia virus, poliovirus, adenovirus, adeno-associated virus, SV40, herpes simplex virus, human immunodeficiency virus, retrovirus (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus) and other recombinant vectors. Other vectors contemplated for eukaryotic target cells include, but are not limited to, the vectors pXT1, pSG5, pSVK3, pBPV, pMSG, and pSVLSV40 (Pharmacia). Additional vectors contemplated for eukaryotic target cells include, but are not limited to, the vectors pCTx-1, pCTx-2, and pCTx-3. Other vectors can be used so long as they are compatible with the host cell.

In some embodiments, a vector has one or more transcription and/or translation control elements. Depending on the host/vector system utilized, any of a number of suitable transcription and translation control elements, including constitutive and inducible promoters, transcription enhancer elements, transcription terminators, etc. can be used in the expression vector. In some embodiments, the vector is a self-inactivating vector that either inactivates the viral sequences or the components of the CRISPR machinery or other elements.

Non-limiting examples of suitable eukaryotic promoters (i.e., promoters functional in a eukaryotic cell) include those from cytomegalovirus (CMV) immediate early, herpes simplex virus (HSV) thymidine kinase, early and late SV40, long terminal repeats (LTRs) from retrovirus, human elongation factor-1 promoter (EF1), a hybrid construct having the cytomegalovirus (CMV) enhancer fused to the chicken beta-actin promoter (CAG), murine stem cell virus promoter (MSCV), phosphoglycerate kinase-1 locus promoter (PGK), and mouse metallothionein-I.

For expressing small RNAs, including guide RNAs used in connection with Cas endonuclease, various promoters such as RNA polymerase III promoters, including for example U6 and H1 promoters, can be advantageous. Descriptions of and parameters for enhancing the use of such promoters are known in art, and additional information and approaches are regularly being described; see, e.g., Ma, H. et al, Molecular Therapy—Nucleic Acids 3, e161 (2014) doi:10.1038/mtna.2014.12.

The expression vector can also contain a ribosome binding site for translation initiation and a transcription terminator. The expression vector can also include appropriate sequences for amplifying expression. The expression vector can also include nucleotide sequences encoding non-native tags (e.g., histidine tag, hemagglutinin tag, green fluorescent protein, etc.) that are fused to the site-directed polypeptide, thus resulting in a fusion protein.

In some embodiments, a promoter is an inducible promoter (e.g., a heat shock promoter, tetracycline-regulated promoter, steroid-regulated promoter, metal-regulated promoter, estrogen receptor-regulated promoter, etc.). In some embodiments, a promoter is a constitutive promoter (e.g., CMV promoter, UBC promoter). In some embodiments, the promoter is a spatially restricted and/or temporally restricted promoter (e.g., a tissue specific promoter, a cell type specific promoter, etc.). In some embodiments, a vector does not have a promoter for at least one gene to be expressed in a host cell if the gene is going to be expressed, after it is inserted into a genome, under an endogenous promoter present in the genome.

### Site-Directed Polypeptide or DNA Endonuclease

The modifications of the target DNA due to NHEJ and/or HDR can lead to, for example, mutations, deletions, alterations, integrations, gene correction, gene replacement, gene tagging, transgene insertion, nucleotide deletion, gene disruption, translocations and/or gene mutation. The process of integrating non-native nucleic acid into genomic DNA is an example of genome editing.

A site-directed polypeptide is a nuclease used in genome editing to cleave DNA. The site-directed polypeptide can be administered to a cell or a patient as either: one or more polypeptides, or one or more nucleic acids encoding the polypeptide.

In the context of a CRISPR/Cas or CRISPR/Cpf1 system, the site-directed polypeptide can bind to a guide RNA that, in turn, specifies the site in the target DNA to which the polypeptide is directed. In embodiments of CRISPR/Cas or CRISPR/Cpf1 systems herein, the site-directed polypeptide is an endonuclease, such as a DNA endonuclease. Such an RNA-guided site-directed polypeptide is also referred to herein as an RNA-guided endonuclease, or RGEN.

Exemplary site-directed polypeptides are described in WO2018002719.

### Target Sequence Selection

In some embodiments, shifts in the location of the 5′ boundary and/or the 3′ boundary relative to particular reference loci are used to facilitate or enhance particular applications of gene editing, which depend in part on the endonuclease system selected for the editing, as further described and illustrated herein.

In a first, non-limiting aspect of such target sequence selection, many endonuclease systems have rules or criteria that guide the initial selection of potential target sites for cleavage, such as the requirement of a PAM sequence motif in a particular position adjacent to the DNA cleavage sites in the case of CRISPR Type II or Type V endonucleases.

In another, non-limiting aspect of target sequence selection or optimization, the frequency of “off-target” activity for a particular combination of target sequence and gene editing endonuclease (i.e., the frequency of DSBs occurring at sites other than the selected target sequence) is assessed relative to the frequency of on-target activity. In some cases, cells that have been correctly edited at the desired locus can have a selective advantage relative to other cells. Illustrative, but non-limiting, examples of a selective advantage include the acquisition of attributes such as enhanced rates of replication, persistence, resistance to certain conditions, enhanced rates of successful engraftment or persistence in vivo following introduction into a patient, and other attributes associated with the maintenance or increased numbers or viability of such cells. In other cases, cells that have been correctly edited at the desired locus can be positively selected for by one or more screening methods used to identify, sort or otherwise select for cells that have been correctly edited. Both selective advantage and directed selection methods can take advantage of the phenotype associated with the correction. In some embodiments, cells can be edited two or more times in order to create a second modification that creates a new phenotype that is used to select or purify the intended population of cells. Such a second modification could be created by adding a second gRNA for a selectable or screenable marker. In some cases, cells can be correctly edited at the desired locus using a DNA fragment that contains the cDNA and also a selectable marker.

In embodiments, whether any selective advantage is applicable or any directed selection is to be applied in a particular case, target sequence selection is also guided by consideration of off-target frequencies in order to enhance the effectiveness of the application and/or reduce the potential for undesired alterations at sites other than the desired target. As described further and illustrated herein and in the art, the occurrence of off-target activity is influenced by a number of factors including similarities and dissimilarities between the target site and various off-target sites, as well as the particular endonuclease used. Bioinformatics tools are available that assist in the prediction of off-target activity, and frequently such tools can also be used to identify the most likely sites of off-target activity, which can then be assessed in experimental settings to evaluate relative frequencies of off-target to on-target activity, thereby allowing the selection of sequences that have higher relative on-target activities. Illustrative examples of such techniques are provided herein, and others are known in the art.

Another aspect of target sequence selection relates to homologous recombination events. Sequences sharing regions of homology can serve as focal points for homologous recombination events that result in deletion of intervening sequences. Such recombination events occur during the normal course of replication of chromosomes and other DNA sequences, and also at other times when DNA sequences are being synthesized, such as in the case of repairs of double-strand breaks (DSBs), which occur on a regular basis during the normal cell replication cycle but can also be enhanced by the occurrence of various events (such as UV light and other inducers of DNA breakage) or the presence of certain agents (such as various chemical inducers). Many such inducers cause DSBs to occur indiscriminately in the genome, and DSBs are regularly being induced and repaired in normal cells. During repair, the original sequence can be reconstructed with complete fidelity, however, in some cases, small insertions or deletions (referred to as “indels”) are introduced at the DSB site.

DSBs can also be specifically induced at particular locations, as in the case of the endonucleases systems described herein, which can be used to cause directed or preferential gene modification events at selected chromosomal locations. The tendency for homologous sequences to be subject to recombination in the context of DNA repair (as well as replication) can be taken advantage of in a number of circumstances, and is the basis for one application of gene editing systems, such as CRISPR, in which homology directed repair is used to insert a sequence of interest, provided through use of a “donor” polynucleotide, into a desired chromosomal location.

Regions of homology between particular sequences, which can be small regions of “microhomology” that can have as few as ten base pairs or less, can also be used to bring about desired deletions. For example, a single DSB is introduced at a site that exhibits microhomology with a nearby sequence. During the normal course of repair of such DSB, a result that occurs with high frequency is the deletion of the intervening sequence as a result of recombination being facilitated by the DSB and concomitant cellular repair process.

In some circumstances, however, selecting target sequences within regions of homology can also give rise to much larger deletions, including gene fusions (when the deletions are in coding regions), which can or cannot be desired given the particular circumstances.

The examples provided herein further illustrate the selection of various target regions for the creation of DSBs designed to insert one or more system components described herein, as well as the selection of specific target sequences within such regions that are designed to minimize off-target events relative to on-target events.

### Nucleic Acid Modifications

In some embodiments, polynucleotides introduced into cells have one or more modifications that can be used independently or in combination, for example, to enhance activity, stability, or specificity; alter delivery; reduce innate immune responses in host cells; or for other enhancements, as further described herein and known in the art.

In certain embodiments, modified polynucleotides are used in the CRISPR/Cas9/Cpf1 system, in which case the guide RNAs (either single-molecule guides or double-molecule guides) and/or a DNA or an RNA encoding a Cas or Cpf1 endonuclease introduced into a cell can be modified, as described and illustrated below. Such modified polynucleotides can be used in the CRISPR/Cas9/Cpf1 system to edit any one or more genomic loci.

Using the CRISPR/Cas9/Cpf1 system for purposes of non-limiting illustrations of such uses, modifications of guide RNAs can be used to enhance the formation or stability of the CRISPR/Cas9/Cpf1 genome editing complex having guide RNAs, which can be single-molecule guides or double-molecule, and a Cas or Cpf1 endonuclease. Modifications of guide RNAs can also or alternatively be used to enhance the initiation, stability or kinetics of interactions between the genome editing complex with the target sequence in the genome, which can be used, for example, to enhance on-target activity. Modifications of guide RNAs can also or alternatively be used to enhance specificity, e.g., the relative rates of genome editing at the on-target site as compared to effects at other (off-target) sites.

Modifications can also or alternatively be used to increase the stability of a guide RNA, e.g., by increasing its resistance to degradation by ribonucleases (RNases) present in a cell, thereby causing its half-life in the cell to be increased. Modifications enhancing guide RNA half-life can be particularly useful in embodiments in which a Cas or Cpf1 endonuclease is introduced into the cell to be edited via an RNA that needs to be translated in order to generate endonuclease, because increasing the half-life of guide RNAs introduced at the same time as the RNA encoding the endonuclease can be used to increase the time that the guide RNAs and the encoded Cas or Cpf1 endonuclease co-exist in the cell.

Modifications can also or alternatively be used to decrease the likelihood or degree to which RNAs introduced into cells elicit innate immune responses. Such responses, which have been well characterized in the context of RNA interference (RNAi), including small-interfering RNAs (siRNAs), as described below and in the art, tend to be associated with reduced half-life of the RNA and/or the elicitation of cytokines or other factors associated with immune responses.

One or more types of modifications can also be made to RNAs encoding an endonuclease that are introduced into a cell, including, without limitation, modifications that enhance the stability of the RNA (such as by increasing its degradation by RNAses present in the cell), modifications that enhance translation of the resulting product (i.e., the endonuclease), and/or modifications that decrease the likelihood or degree to which the RNAs introduced into cells elicit innate immune responses.

Combinations of modifications, such as the foregoing and others, can likewise be used. In the case of CRISPR/Cas9/Cpf1, for example, one or more types of modifications can be made to guide RNAs (including those exemplified above), and/or one or more types of modifications can be made to RNAs encoding Cas endonuclease (including those exemplified above).

Exemplary modified nucleic acids are described in WO2018002719.

### Delivery

In some embodiments, any nucleic acid molecules used in the methods provided herein, e.g., a nucleic acid encoding a genome-targeting nucleic acid of the disclosure and/or a site-directed polypeptide are packaged into or on the surface of delivery vehicles for delivery to cells. Delivery vehicles contemplated include, but are not limited to, nanospheres, liposomes, quantum dots, nanoparticles, polyethylene glycol particles, hydrogels, and micelles. As described in the art, a variety of targeting moieties can be used to enhance the preferential interaction of such vehicles with desired cell types or locations.

Introduction of the complexes, polypeptides, and nucleic acids of the disclosure into cells can occur by viral or bacteriophage infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Exemplary delivery methods and reagents are described in WO2018002719.

The present disclosure has been described above with reference to specific alternatives. However, other alternatives than the above described are equally possible within the scope of the disclosure. Different method steps than those described above, may be provided within the scope of the disclosure. The different features and steps described herein may be combined in other combinations than those described.

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those of skill within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

In addition, where features or aspects of the disclosure are described in terms of Markush groups, those skilled in the art will recognize that the disclosure is also thereby described in terms of any individual member or subgroup of members of the Markush group.

Any of the features of an alternative of the first through eleventh aspects is applicable to all aspects and alternatives identified herein. Moreover, any of the features of an alternative of the first through eleventh aspects is independently combinable, partly or wholly with other alternatives described herein in any way, e.g., one, two, or three or more alternatives may be combinable in whole or in part. Further, any of the features of an alternative of the first through eleventh aspects may be made optional to other aspects or alternatives. Although described above in terms of various example alternatives and implementations, it should be understood that the various features, aspects and functionality described in one or more of the individual alternatives are not limited in their applicability to the particular alternative with which they are described, but instead may be applied, alone or in various combinations, to one or more of the other alternatives of the present application, whether or not such alternatives are described and whether or not such features are presented as being a part of a described alternative. Thus, the breadth and scope of the present application should not be limited by any of the above-described example alternatives.

All references cited herein are incorporated herein by reference in their entirety. To the extent publications and patents or patent applications incorporated by reference contradict the disclosure contained in the specification, the specification is intended to supersede and/or take precedence over any such contradictory material. To the extent publications and patents or patent applications incorporated by reference herein contradict the disclosure contained in the specification, the specification is intended to supersede and/or take precedence over any such contradictory material.

The details of one or more embodiments of the disclosure are set forth in the accompanying description below. Any materials and methods similar or equivalent to those described herein can be used in the practice or testing of the present disclosure. Other features, objects and advantages of the disclosure will be apparent from the description. In the description, the singular forms also include the plural unless the context clearly dictates otherwise. Unless defined otherwise, all technical and scientific terms used herein have the same meaning as commonly understood by one of ordinary skill in the art to which this disclosure belongs. In the case of conflict, the present description will control.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Some embodiments of the disclosures provided herewith are further illustrated by the following non-limiting examples.

### Examples

### Example 1: NHEJ-Mediated Targeted Integration in K562 Cells

This study was carried out to determine in K562 cells the feasibility of NHEJ-mediated targeted integration stimulated with and without donor cleavage. Plasmid donor vectors with a GFP expression cassette dependent upon the endogenous PPP1R12C promoter for expression were cloned with and without CRISPR/Cas9 recognition sites for in vivo donor cleavage.

In these experiments, three hundred thousand K562 cells were nucleofected with 1-cut plasmid donor (pCR2.1-TOPO-psAAVS1-SA-P2A-H2Bj-Venus-BGHpA-psSCRAM) at 1.50, 2.75, and 3.50 pmols or 0-cut plasmid donor (pCR2.1-TOPO-psSCRAM-SA-P2A-H2Bj-Venus-BGHpA-psSCRAM) at 3.50 pmols on day −1. pCR2.1-TOPO: vector plasmid backbone; psSCRAM: scrambled protospacer sequence with PAM; psAAVS1: same protospacer sequence as the genomic DNA sequence the Cas9/gRNA RNP cleaves with the PAM; SA: splice acceptor; P2A: peptide cleavage signal sequence; H2Bj-Venus: modified GFP fused to the histone H2B that stabilizes GFP in the nucleus; and BGHpA: bovine growth hormone polyadenylation sequence. Twenty-four hours later, the K562 cells in each condition were nucleofected with or without RNPs. RNPs were a complex of 12 pmols Cas9 (Feldan) and 60 pmols AAVS1 guide RNA (Axolabs). The standard nucleofection protocol by Lonza 4D Nucleofector® for K562 cell line was used. Cells were analyzed by flow cytometry on days 7 (D7) and 14 (D14) post-nucleofection, and genomic DNA (gDNA) was collected on D7 for molecular analysis of integration.

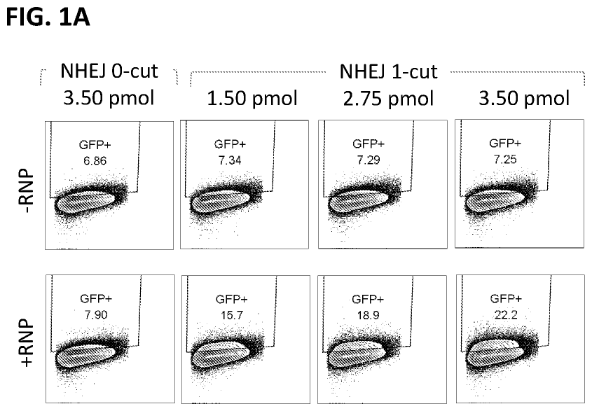

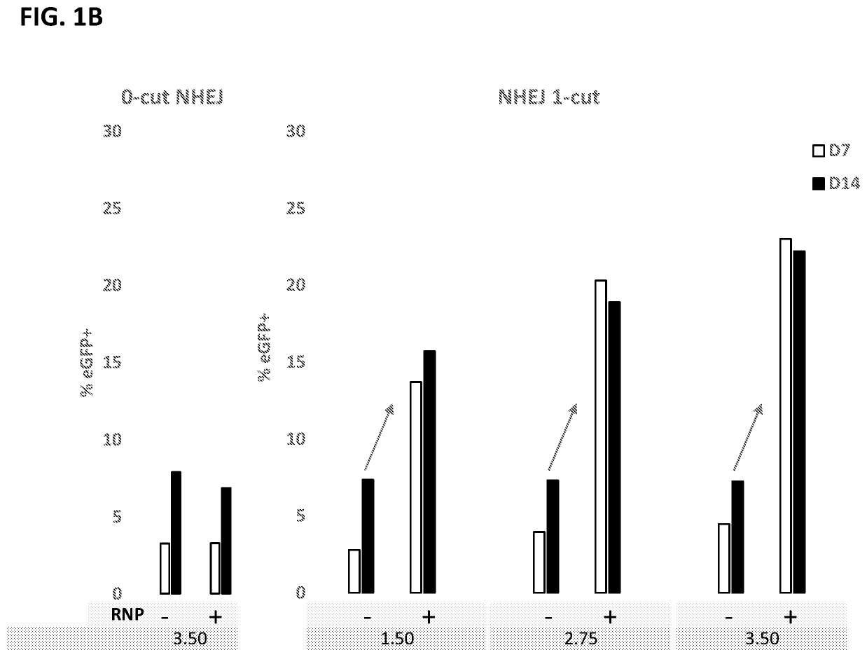

The flow cytometry results are shown in FIGS. 1A and 1B. The editing efficiencies as determined by GFP⁺ signal were as follows: NHEJ 0-cut 3.5 pmols −RNP: D7=3.25%, D14=7.9%; NHEJ 0-cut 3.5 pmols +RNP: D7=3.28%, D14=6.86%; NHEJ 1-cut 1.50 pmols −RNP: D7=2.78%, D14=7.34%; NHEJ 1-cut 1.50 pmols +RNP: D7=13.7%, D14=15.7%; NHEJ 1-cut 2.75 pmols −RNP: D7=3.95%, D14=7.29%; NHEJ 1-cut 2.75 pmols +RNP: D7=20.3%, D14=18.9%; NHEJ 1-cut 3.50 pmols −RNP: D7=4.46%, D14=7.25%; NHEJ 1-cut 3.50 pmols +RNP: D7=23.0%, D14=22.2%. These results demonstrate improved editing efficiency with donor cleavage.

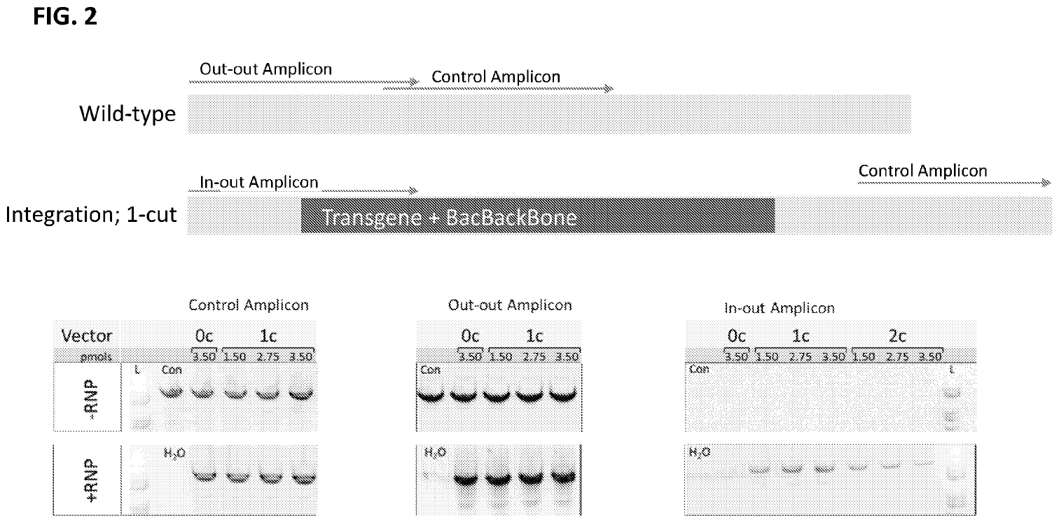

The gDNA extracted on D7 was used for in-out PCR for integration detection (see schematic in FIG. 2). gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11); 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15)); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA; 1 μl GC-enhancer; and H2O up to 20 μl total. As shown in FIG. 2, all samples had the out-out band as expected, but only samples treated with both Cas9/gRNA RNP and donor plasmid with at least one cut site targeted by the RNP showed integration of the transgene as evidenced by the in-out PCR band.

### Example 2: T-Cells as a Model for NHEJ-Vs HDR-TI in Dividing Vs Non-Dividing Cells

This study was carried out to determine in human CD3⁺ T cells the feasibility of NHEJ-mediated targeted integration versus HDR-mediated targeted integration in dividing versus non-dividing cells. Purified T cells in ex vivo culture are naturally quiescent and do not undergo HDR efficiently unless stimulated to initiate growth and expansion. The following describes both HDR-mediated targeted integration and NHEJ-mediated targeted integration in both stimulated and unstimulated T cells.

### Post-STIM (Dividing) T-Cell Targeted Integration

TABLE 1

| | −RNP | +RNP |
|---|---|---|
| 1 | Manipulation Control | RNP only control |
| 2 | NHEJ-TI virus | NHEJ-TI virus |
| 3 | HDR-TI virus | HDR-TI virus |
| 4 | Both virus | Both Virus |

Conditions in duplicate

Pan CD3⁺ T-cells were isolated via StemCell Technologies kit (17951), cultured in AIMV (Invitrogen 12055083)+5% CTS Immune Cell Serum Replacement (SR) (Invitrogen A2596101), and activated using T Cell Activation/Expansion Kit (Miltenyi Biotec 130-093-627) with IL-2 100 ng/ml (PeproTech) and IL-7 100 ng/ml (PeproTech).

NHEJ-TI virus (SEQ ID NO: 6) from VBL (CBGU004) was used at MOI of 50,000 vg/cell and HDR-TI virus (SEQ ID NO: 7) from VBL (CBGGU014) was used at MOI of 50,000 vg/cell. RNPs included GeneArt v2 Cas9 1 pmol/10,000 cells and Synthego gRNA (SEQ ID NO: 8) 2.5 pmol/10,000 cells.

Lonza 4D Nucleofector® was used with Primary Cell P3 nucleofector solution (Lonza V4XP-3032) and nucleofection protocol EO-115 at manufacturer's recommendation.

### Cell Culture

In these experiments, two hundred thousand cells/condition (3.2 million cells total) were thawed and DMSO was washed out (1 ml frozen cells thawed and diluted in 10 ml media). Cells were spun at 350×g for 10 minutes, supernatant was aspirated, and tubes were tapped to resuspend cells. Media was added at one million cells/ml with activation beads, and cells were cultured for 48 hours.

### Bead Depletion

On day two post-thaw, cells were collected into 50 ml conical tubes and pipetted vigorously to dissociate clumped cells. Tubes containing cells were placed on a magnetic stand and incubated for 10 minutes, after which supernatants were gently pipetted into other 50 ml conical tubes. The tubes containing cells were again placed on a magnetic stand and incubated for 10 minutes, followed by gently pipetting supernatants into other 50 ml conical tubes. Tubes were centrifuged at 350×g for 10 minutes, supernatant was aspirated off, and cells were resuspended at one million cells/ml and cultured for at least two hours before nucleofection.

### RNP Nucleofection

Cells were collected into 50 ml conical tubes and centrifuged at 350×g for 10 minutes, during which time RNP complexes were prepared. Previously aliquoted RNP complex was thawed and P3 nucleofection reagent +RNP master mix at 20:50 pmols Cas9:gRNA/10 μl nucleofection reagent was prepared. Ten μl nucleofection reagent/well was aliquoted to 16-well nucleofection strips in appropriate well/condition. Collected cells were resuspended in P3 nucleofection reagent at 200,000 cells/10 μl (20,000 cells/μ1) and 10 μl cell-containing nucleofection reagent was distributed to each well of the 16-well nucleofection strips for a total volume of 20 μl.

### Viral Transduction

Prior to nucleofection, viral transduction media was prepared. A virus-containing master mix of serum-free media was prepared as follows: a. 80 μl total volume with 50,000 vg/cell virus; b. 320 μl (+ extra) with 40 billion (vg+extra). A media-only control was also prepared. Eighty μl transduction media or control was aliquoted to wells of a 96-well plate.

Following nucleofection, 80 μl of the transduction media was aspirated with a 100 μl pipette and gently transferred into wells of the 16-well nucleofection strips. One hundred μl of transduction media/nucleofection media mixture was aspirated out and pipetted back into the corresponding wells of the 96-well plate. Cells (now at 2×10⁶ cells/ml) were incubated for two hours to allow for transduction, then transferred into wells of a 24-well plate with 1 ml media. Cells were cultured for up to two weeks and analyzed by flow cytometry on days 2 (D2), 4 (D4), 7 (D7), and 14 (D14). Genomic DNA was collected on day 2 (D2) to determine cutting efficiency and on day 7 (D7) to determine donor integration.

### NO-STIM (Non-Dividing) T-Cell Targeted Integration

TABLE 2

| | −RNP | +RNP |
|---|---|---|
| 1 | Manipulation Control | RNP only control |
| 2 | NHEJ-TI virus | NHEJ-TI virus |
| 3 | HDR-TI virus | HDR-TI virus |
| 4 | Both virus | Both Virus |

### Conditions in Triplicate

Pan CD3⁺ T-cells were isolated via StemCell Technologies kit (17951), and cultured in AIMV (Invitrogen 12055083)+5% CTS Immune Cell SR (Invitrogen A2596101) with IL-2 100 ng/ml (PeproTech), IL-7 100 ng/ml (PeproTech), IL-15 100 ng/ml (PeproTech), SCF 100 ng/ml (PeproTech), and FLT3L 100 ng/ml (PeproTech).

NHEJ-TI virus (SEQ ID NO: 6) from VBL (CBGU004) was used at MOI of 50,000 vg/cell and HDR-TI virus (SEQ ID NO: 7) from VBL (CBGGU014) was used at MOI of 50,000 vg/cell. RNPs included GeneArt v2 Cas9 1 pmol/10,000 cells and Synthego gRNA (SEQ ID NO: 8) 2.5 pmol/10,000 cells.

Lonza 4D Nucleofector® was used with Primary Cell P3 nucleofector solution (Lonza V4XP-3032) and nucleofection protocol EO-115 at manufacturer's recommendation.

### Cell Culture

In these experiments, five million cells/condition (120 million cells total) were thawed and DMSO was washed out (5 ml frozen cells thawed and diluted in 50 ml media). Cells were spun at 350×g for 10 minutes, supernatant was aspirated, and tubes were tapped to resuspend cells. Media was added at two to five million cells/ml, and cells were cultured for 24 hours.

### RNP Nucleofection

Cells were collected into 50 ml conical tubes and centrifuged at 350×g for 10 minutes, during which time RNP complexes were prepared. Previously aliquoted RNP complex was thawed and P3 nucleofection reagent +RNP master mix at 200:500 pmols Cas9:gRNA/50 μl nucleofection reagent was prepared. Fifty μl nucleofection reagent was aliquoted to 100-μl nucleofection cuvettes for appropriate conditions. Collected cells were resuspended in P3 nucleofection reagent at 2,000,000 cells/50 μl and 50 μl cell-containing nucleofection reagent was distributed to each nucleofection cuvette for a total volume of 100 μl.

### Viral Transduction

Prior to nucleofection, viral transduction media was prepared. A virus-containing master mix of serum-free media was prepared as follows: a. 900 μl total volume with 50,000 vg/cell virus; b. 4800 μl (+ extra) with 100 billion (vg+extra). A media-only control was also prepared. Nine hundred μl transduction media or control was aliquoted to wells of a 24-well plate.

Following nucleofection, ˜500 μl of the transduction media was aspirated with a transfer pipette and gently transferred into the nucleofection cuvettes. All of the transduction media/nucleofection media mixture was aspirated out and pipetted back into the corresponding wells of the 24-well plate. Cells were cultured for up to two weeks and analyzed by flow cytometry on days 2 (D2), 4 (D4), 7 (D7), and 14 (D14). Genomic DNA was collected on day 2 (D2) to determine cutting efficiency and on day 7 (D7) to determine donor integration.

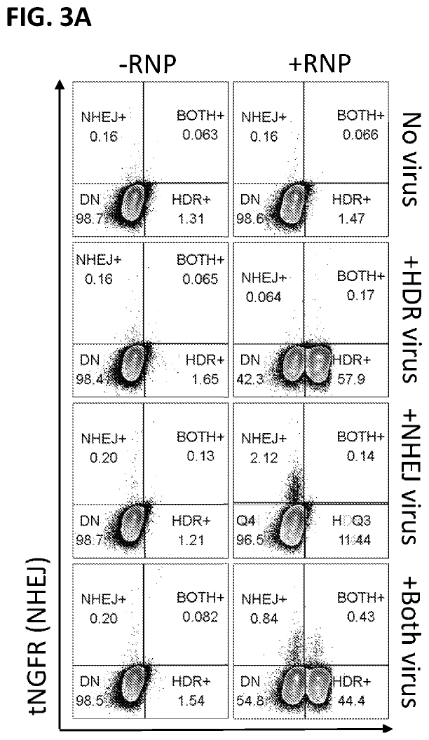

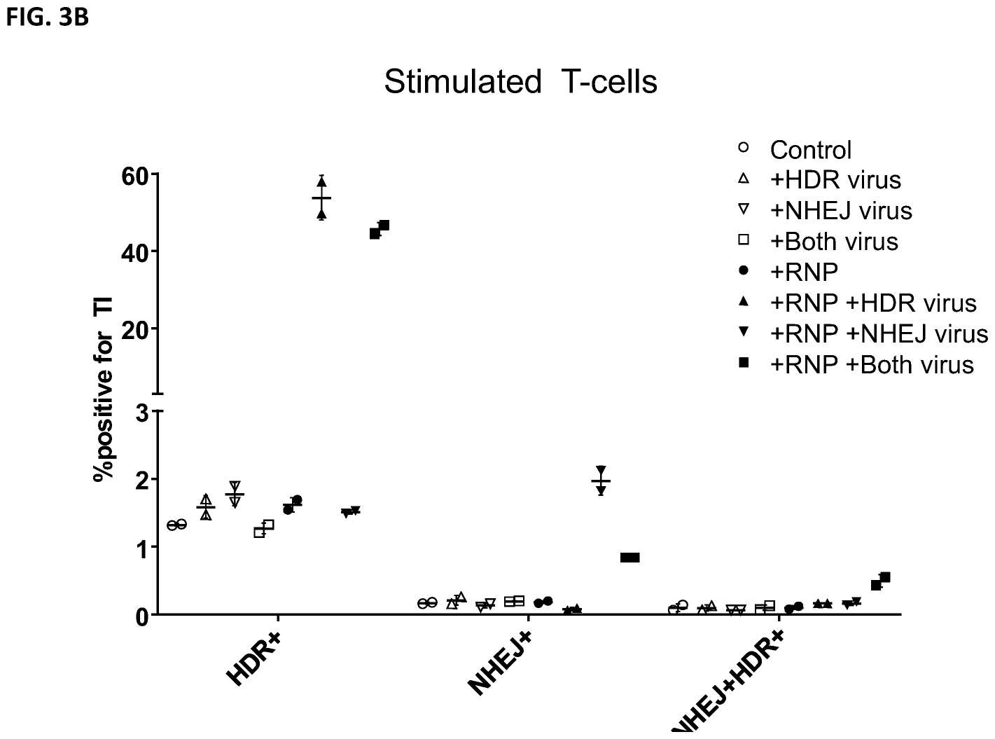

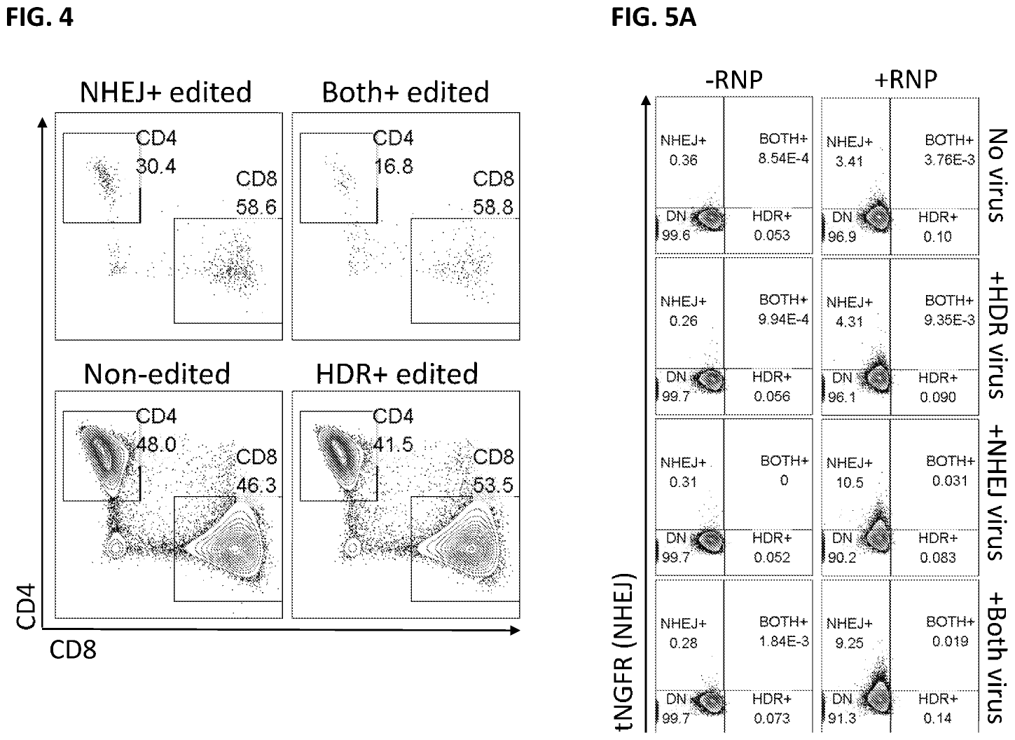

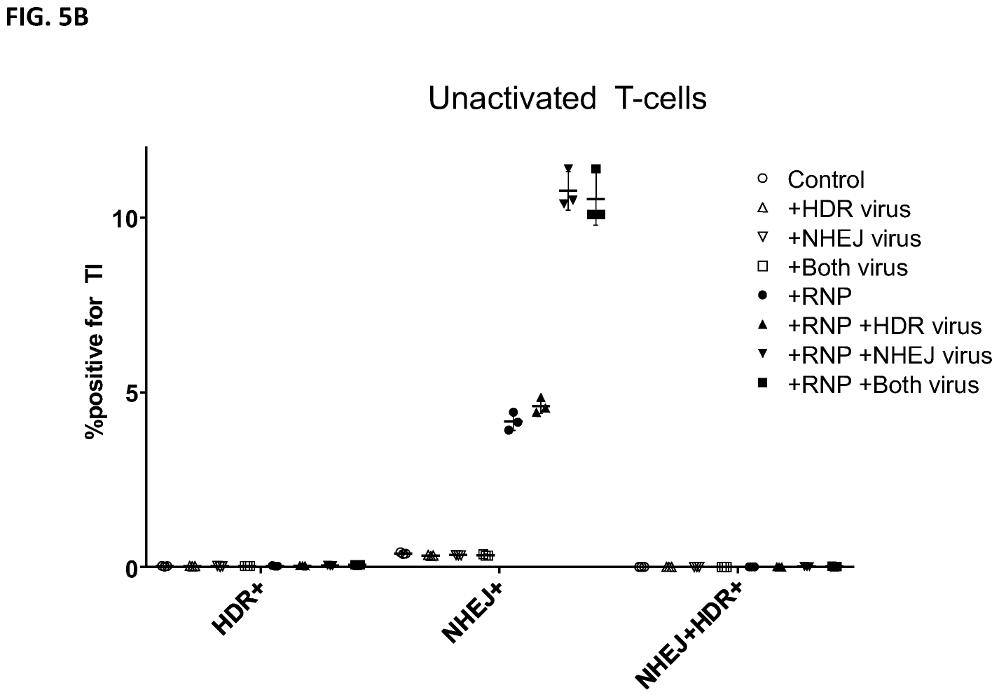

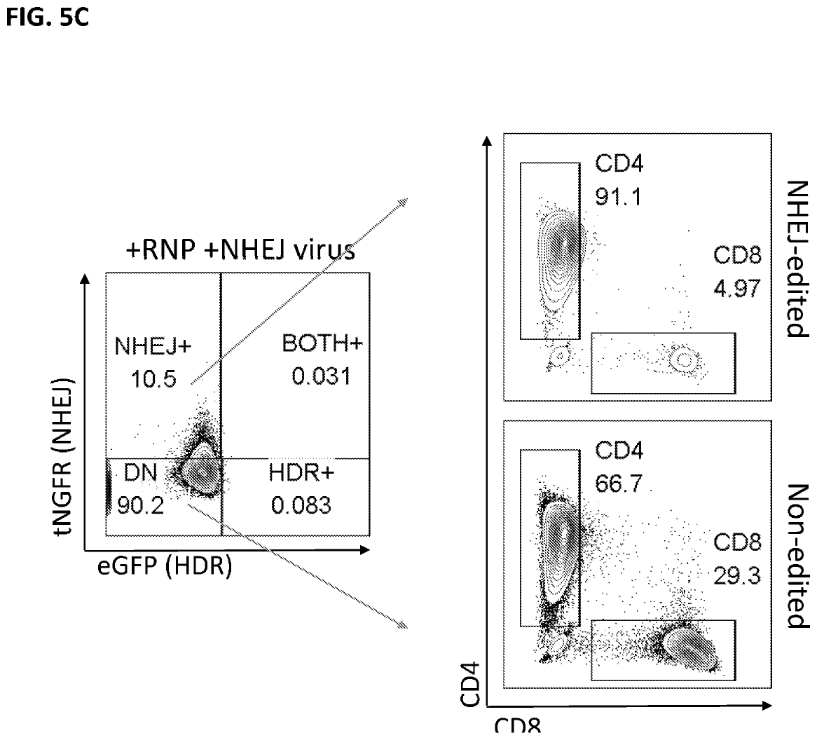

Results are shown in FIGS. 3A, 3B, 4, 5A, 5B, and 5C. HDR-TI was favored in the activated T cells (FIGS. 3A and 3B), whereas NHEJ-TI was favored in the non-activated T cells (where these was no detectable HDR-TI; FIGS. 5A and 5B). Further, as shown in FIG. 5C NHEJ-edited non-activated T cells were predominantly CD4⁺ (91.1% CD4⁺ as compared to 4.97% CD8⁺).

### Example 3: Testing NHEJ-Mediated TI in CD34 Cells

This study was carried out to determine in human CD34⁺ cells, the feasibility of NHEJ-mediated targeted integration stimulated with and without in vivo donor cleavage. In this study, two protocols were compared to determine if order of manipulation affects yields of targeted integration and viability/cellularity. HSCs were treated with scAAV6 carrying either a 2-cut donor (SEQ ID NO: 5) or a 0-cut donor (SEQ ID NO: 4) and RNP.

TABLE 3

| | | Nucleofection |
|---|---|---|
| Group # | Group Condition | order |
| S1 | Stem Cell Technologies cells: Control | Mock treatment |
| S2 | Stem Cell Technologies cells: RNP only | +RNP only |
| | control | |
| S3 | Stem Cell Technologies cells: AAV-0 cut only | +AAV-0 only |
| | control | |
| S4 | Stem Cell Technologies cells: AAV-2 cut only | +AAV-2 only |
| | control | |
| S5 | Stem Cell Technologies cells: P2 AAV-0 cut | +RNP +AAV-0 |
| S6 | Stem Cell Technologies cells: P2 AAV-2 cut | +RNP +AAV-2 |
| S7 | Stem Cell Technologies cells: P1 AAV-0 cut | +AAV-0 +RNP |
| S8 | Stem Cell Technologies cells: P1 AAV-2 cut | +AAV-2 +RNP |
| A1 | AllCell Cells: Control | Mock treatment |
| A2 | AllCell Cells: AAV-2 cut only control | +AAV-2 only |
| A3 | AllCell Cells: CTX AAV-2 cut | +AAV-2 +RNP |

Two hundred thousand CD34⁺ cells were used per condition (from Stem Cell Technologies or AllCells). scAAV6 0-cut NHEJ vector (SEQ ID NO: 4) or 2-cut NHEJ vector (SEQ ID NO: 5) (Vector Biolabs) were used at an MOI of 20,000 vg/cell. RNPs included 24 pmols Cas9 (Feldan) to 120 pmols AAVS1 guide RNA (SEQ ID NO: 8) (Axolabs).

Lonza 4D Nucleofector® was used with Primary Cell P3 nucleofector solution (Lonza V4XP-3032) and nucleofection protocol DZ-100 at manufacturer's recommendation.

### Cell Culture

Aliquoted media and cytokines were thawed immediately before use. Thawed cytokines and small molecules were added to StemSpan SFEM II to create complete media, which was warmed to 37° C. Vials of hCD34⁺ sufficient for 200,000 cells/condition were thawed, and cells were transferred to 15 ml conical and total volume was brought up to 15 ml with CD34⁺ culture media. Cells were spun at 90×g for 9 minutes, supernatant was aspirated, and cells were resuspended in 1 ml CD34⁺ culture media. The total volume was brought up to at least 20 ml in low-binding T75 flasks (at most 200,000 cells/ml), and cells were allowed to rest for at least 24 hours.

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube. Enough RNP was thawed for subsequent experiments. Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of nucleofection strips and nucleofection reagent was added up to 10 μl volume. Culture plates were prepared by adding 1 ml complete media to each well of a 24-well plate. Cell supernatants were aspirated or decanted, and cells were resuspend in 1/2 volume per sample of Buffer P3+Supplement. Ten μl of the cells in nucleofection reagent was added to each well, and the nucleofector was run according to manufacturer's protocol. After nucleofection, 80 μl pre-warmed complete media was immediately added to each well, and cells were gently pipetted cells into each well of the prepared culture plates.

### AAV Infection

AAV were thawed on ice. Thawed cytokines and small molecules were added to StemSpan SFEM II to create complete media immediately before use, which was warmed to 37° C. Cells were collected into 15 ml conical tubes, and 100 μl (1:100 of total) cells were taken and diluted to 1 ml (1:10 dilution) for measuring viability and cell concentration using a Vi-Cell XR. Cells were spun at 90×g for 9 minutes, during which time AAV dilutions were prepared in low-binding protein tubes. Culture plates were prepared by adding 50 μl complete media to each well of a 96-well plate, and corresponding amounts of the AAV dilutions were added to each well. Supernatant was decanted, and cells were resuspended in 50 μl times the number of conditions of complete media. Fifty μl of the cells were added to each well, and cells were placed back in a humidified 37° C. normoxic incubator for two hours. Cells were then collected into 1.5 ml sterile eppendorf tubes and spun at 90×g for 10 minutes, during which time culture plates were prepared by adding 0.7 ml complete media in each well of a 24-well plate. Supernatant was decanted and cells were resuspended in 300 μl complete culture media and transferred to the prepared culture plate.

### Condition-Specific Protocols

### Protocol 1 (P1)

Protocol 1 (P1) was carried out with AAV treatment first, followed by RNP treatment. Cells were treated with AAV, and two hours later were collected into 1.5 ml sterile Eppendorf tubes. Cells were spun at 90×g for 10 minutes, during with time nucleofection reagents and RNP were prepared, as well as 24-well culture plates with 900 μl complete media per well. Cells were resuspended in 20 μl P3+/−RNP, and 100 μl of cells were transferred to the prepared 24-well culture plates. Cells were cultured in a normoxic incubator for up to +5 days, adding media every two days.

### Protocol 2 (P2)

Protocol 2 (P2) was carried out with RNP treatment first, followed by AAV treatment. Cells were nucleofected with or without RNP, and 100 μl of cells were transferred to prepared culture plates. The cells were allowed to rest for one hour, followed by treatment with AAV for two hours. Cells were then collected, spun, and resuspended in fresh media, and cultured in a normoxic incubator for up to +5 days, adding media every two days.

### Data Collection

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: 1. Denature: 95° C. 00:010:00; 2. Denature: 95° C. 00:00:15; 3. Annealing: 60° C. 00:00:15; 4. Extension: 72° C. 00:02:00; Repeat steps 2-4 ×35 cycles; 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11); 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15)); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA; 1 μl GC-enhancer; and H₂O up to 20 μl total.

### TOPO-TA Cloning

TOPO-TA cloning was carried out using 4 μl in-out PCR amplicons, 1 μl salt solution, and 1 μl TOPO-TA vector (pCRTM2.1-TOPO® vector). Reactions were incubated at 25° C. for 15 minutes following by chilling on ice.

TOP10 chemically competent cells were thawed on ice, and 2 μl TOPO-TA Cloning products were added to the thawed TOP10 chemically competent cells. Cell suspensions were mixed by gentle tapping and incubated on ice for 5 minutes followed by heat shock at 42° C. for 45 seconds. Cells were then incubated on ice for 5 minutes, 250 μl SOC media was added, and cells were incubated at 37° C. for 30 minutes in a bacteria shaker. One hundred fifty μl of cells were plated on X-gal-coated carbenicillin agar plates and incubated overnight. White colonies were selected for plasmid growth and sequencing.

### Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system.

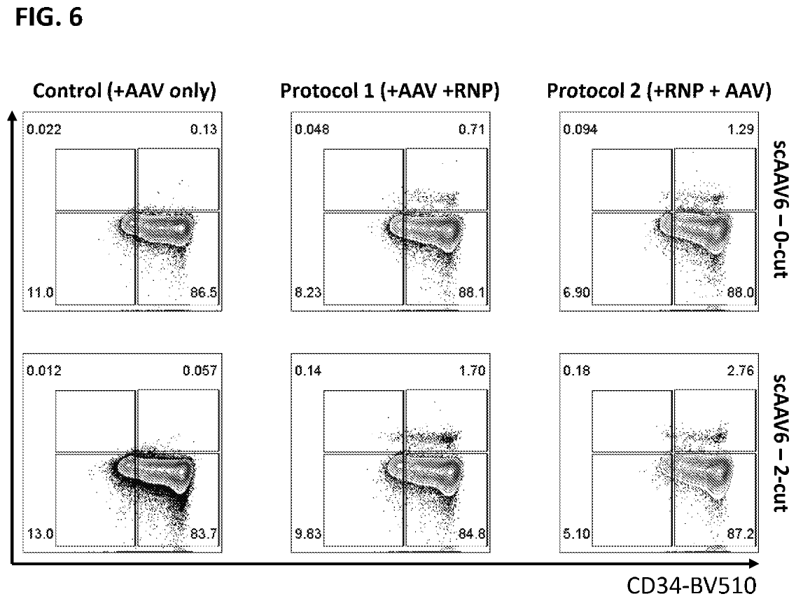

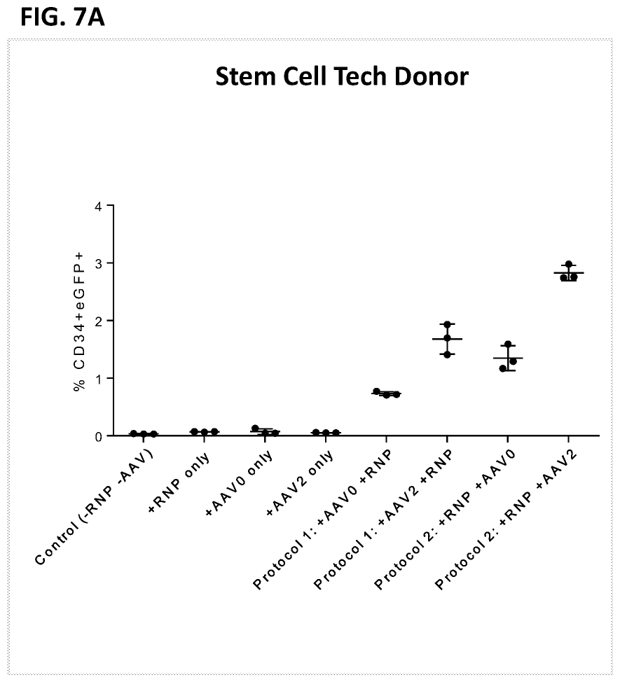

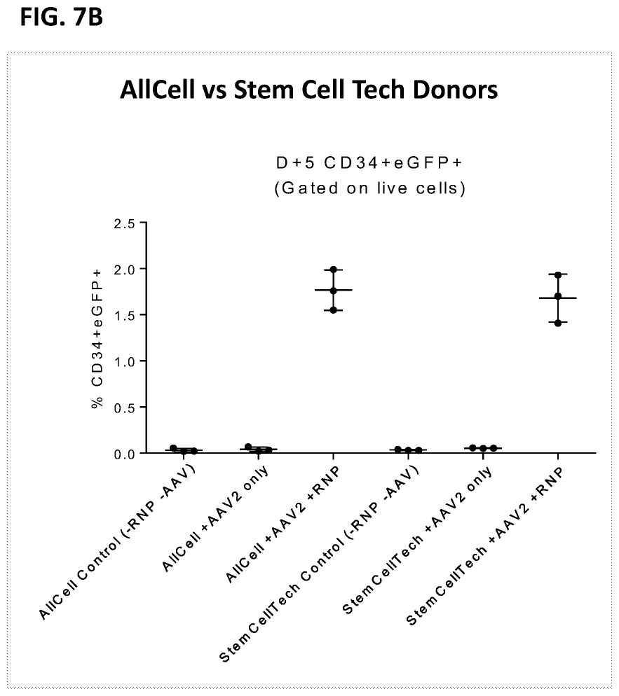

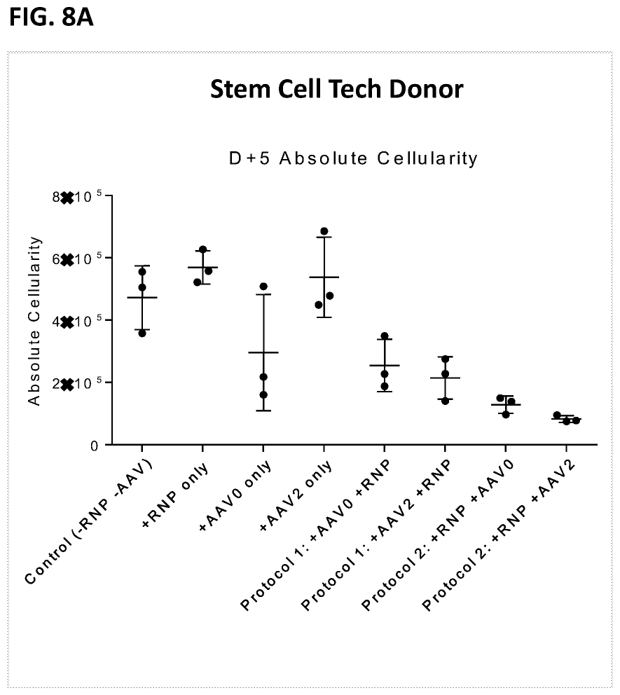

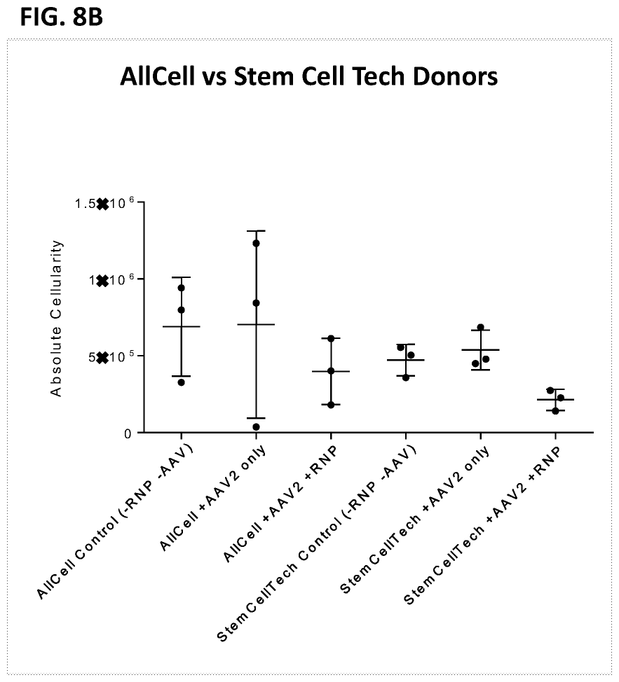

Results are shown in FIGS. 6, 7A, 7B, 8A, and 8B. For both P1 and P2, editing efficiency was higher for the 2-cut donor than the 0-cut donor, and P2 showed greater editing efficiency as compared to P1 for corresponding conditions (FIGS. 6 and 7A), further demonstrating the advantage of donor cleavage and suggesting that RNP treatment first followed by AAV treatment leads to more efficient targeted integration.

### Example 4: Testing NHEJ-Mediated TI in CD34 Cells: SCGM Vs SFEM-II Media, Hypoxia Vs Normoxia, D1-Edit Vs D2-Edit

This study was carried out to determine in human CD34⁺ cells the feasibility of NHEJ-mediated targeted integration stimulated with and without in vivo donor cleavage. Here, it was examined whether the media or 02 levels had an effect on NHEJ-TI. One day (D1) vs two day (D2) resting period conditions were also tested, as both P2 and P1 rest and pre-stimulation HSCs for 48 hours before editing via HDR-mediated targeted integration.

TABLE 4
D1-edit Conditions

| Normoxia | No RNP | +RNP |
|---|---|---|
| SCGM Media: No virus | Mock Nucleofection | RNP Nucleofection |
| SCGM Media: 2-cut scAAV6 | −RNP | +RNP |
| SFEM-II Media: No virus | Mock Nucleofection | RNP Nucleofection |
| SFEM-II Media: 2-cut | −RNP | +RNP |
| scAAV6 | | |
| Hypoxia | | |
| SCGM Media: No virus | Mock Nucleofection | RNP Nucleofection |
| SCGM Media: 2-cut scAAV6 | −RNP | +RNP |
| SFEM-II Media: No virus | Mock Nucleofection | RNP Nucleofection |
| SFEM-II Media: 2-cut | −RNP | +RNP |
| scAAV6 | | |

TABLE 5
D2-edit Conditions

| Normoxia | No RNP | +RNP |
|---|---|---|
| SCGM Media: No virus | Mock Nucleofection | RNP Nucleofection |
| SCGM Media: 2-cut scAAV6 | −RNP | +RNP |
| SFEM-II Media: No virus | Mock Nucleofection | RNP Nucleofection |
| SFEM-II Media: 2-cut | −RNP | +RNP |
| scAAV6 | | |
| Hypoxia | | |
| SCGM Media: No virus | Mock Nucleofection | RNP Nucleofection |
| SCGM Media: 2-cut scAAV6 | −RNP | +RNP |
| SFEM-II Media: No virus | Mock Nucleofection | RNP Nucleofection |
| SFEM-II Media: 2-cut | −RNP | +RNP |
| scAAV6 | | |

**Experiments done in triplicate

In these experiments, 200,000 CD34⁺ cells/condition (from Stem Cell Technologies) were used. The cells were cultured in SCGM (CellGenix, GMP grade) or SFEM-II (Stem Cell Technologies) with SCF 100 ng/ml; TPO 100 ng/ml; FLT3L 100 ng/ml; and IL6 100 ng/ml in either hypoxic conditions (5% 02, 5% CO₂, 90% N2) or normoxic conditions (ATM 02, 5% CO₂, 90% N2).

RNPs included 24 pmols Cas9 (Feldan) to 120 pmols AAVS1 guide RNA (SEQ ID NO: 8) (Axolabs). scAAV6 2-cut NHEJ vector (SEQ ID NO: 5) (Vector Biolabs) was used at an MOI of 20,000 vg/cell with two hour incubation at 2×10⁶ cells/ml followed by transfer to 0.9 ml fresh media.

Lonza 4D Nucleofector® was used with Primary Cell P3 nucleofector solution (Lonza V4XP-3032) and nucleofection protocol DZ-100 at manufacturer's recommendation.

### Cell Culture

Aliquoted media and cytokines were thawed immediately before use. Thawed cytokines and small molecules were added to SCGM or SFEM II to create complete media, which was warmed to 37° C. Vials of hCD34⁺ sufficient for 200,000 cells/conditions were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cell s/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube (enough RNP was thawed for subsequent experiments). Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of the nucleofection strips and nucleofection reagent was added up to 10 μl volume. Culture plates were prepared by adding 1 ml complete media to each well of a 24-well plate. Cell supernatants were aspirated or decanted and cells were resuspended in 1/2 volume per sample of Buffer P3+Supplement. Ten μl of the cells in nucleofection reagent were added to each well, and the nucleofector was run according to manufacturer's protocol. After nucleofection 80 μl pre-warmed complete media was immediately added to each well and cells were gently pipetted into each well of the prepared culture plates.

### AAV Infection

AAV were thawed on ice. Thawed cytokines and small molecules were added to SCGM or SFEM II to create complete media immediately before use, which was warmed to 37° C., during which time AAV dilutions were prepared in low-binding protein tubes. 96-well culture plates were prepared, and 80 μl complete media and corresponding amounts of the AAV dilutions were added to each well. 100 μl of nucleofected cells (with 80 μl rescue media added) were transferred from the 16-well strips to the prepared culture plates, and cells were placed back in a humidified 37° C. hypoxic incubator for two hours. Cells were then collected, during which time culture plates were prepared by adding 0.7 ml complete media in each well of a 24-well plate. Cell supernatants were decanted and cells were resuspended and transferred to the prepared 24-well culture plate. The 96-well culture plates were washed with 200 μl fresh media and vigorous pipetting, and media was transferred to corresponding wells of the 24-well plate.

Cells were then cultured for four days, after which they were collected and pelleted in 1.5 ml tubes. gDNA was collected using the Machery-Nagel genomic DNA extraction kit according to manufacturer's protocol.

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: 1. Denature: 95° C. 00:010:00; 2. Denature: 95° C. 00:00:15; 3. Annealing: 60° C. 00:00:15; 4. Extension: 72° C. 00:02:00 (Repeat steps 2-4 ×35 cycles); 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11) 1 μl; 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15) 1 μl); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA, 1 μl GC-enhancer, and H₂O up to 20 μl total.

Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system. The flow cytometry CD34 ex vivo panel included CD34 BV510; CD90 PerCP-Cy5.5; CD38 APC; CD45RA PE; and Ghost Red 780 viability dye.

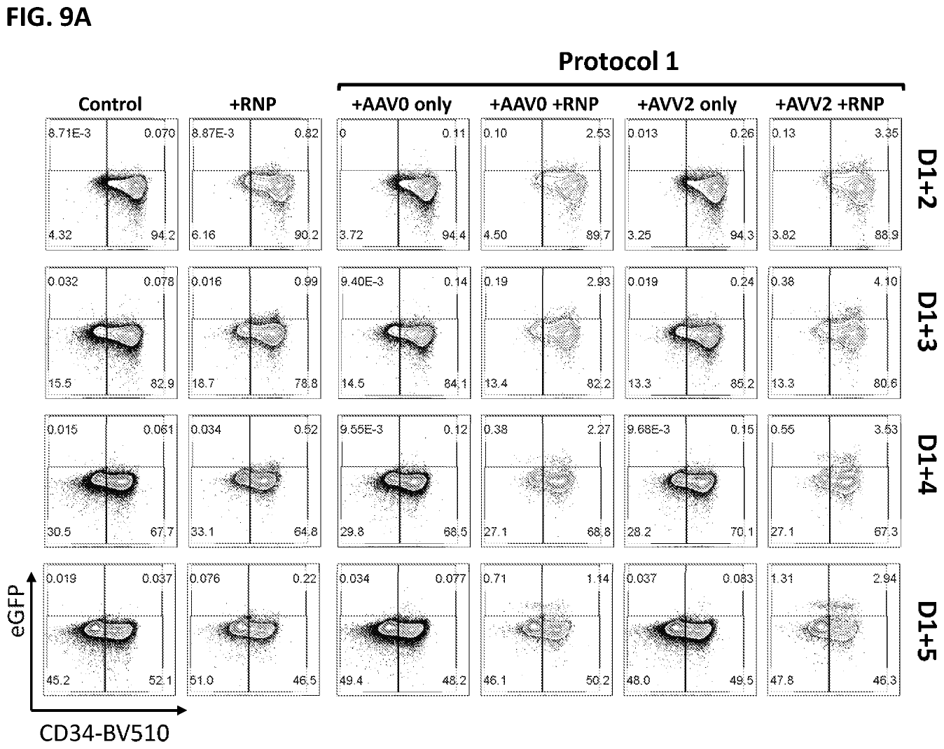

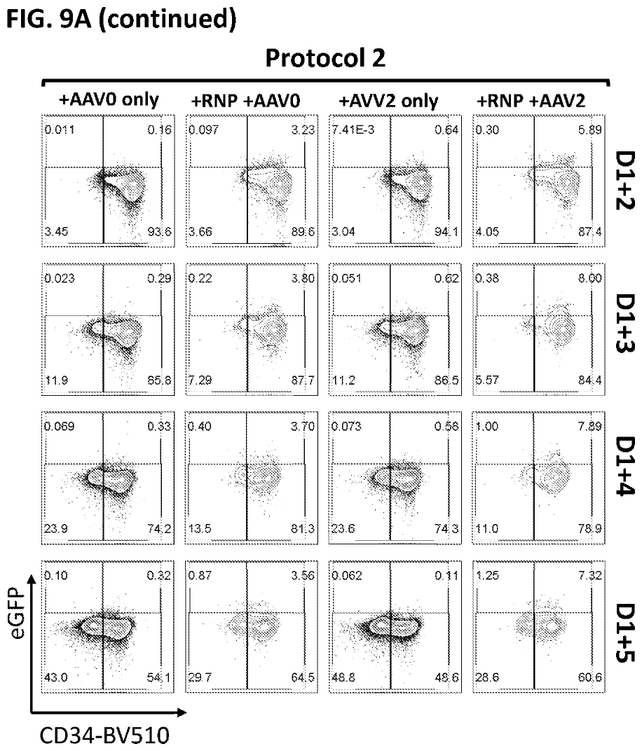

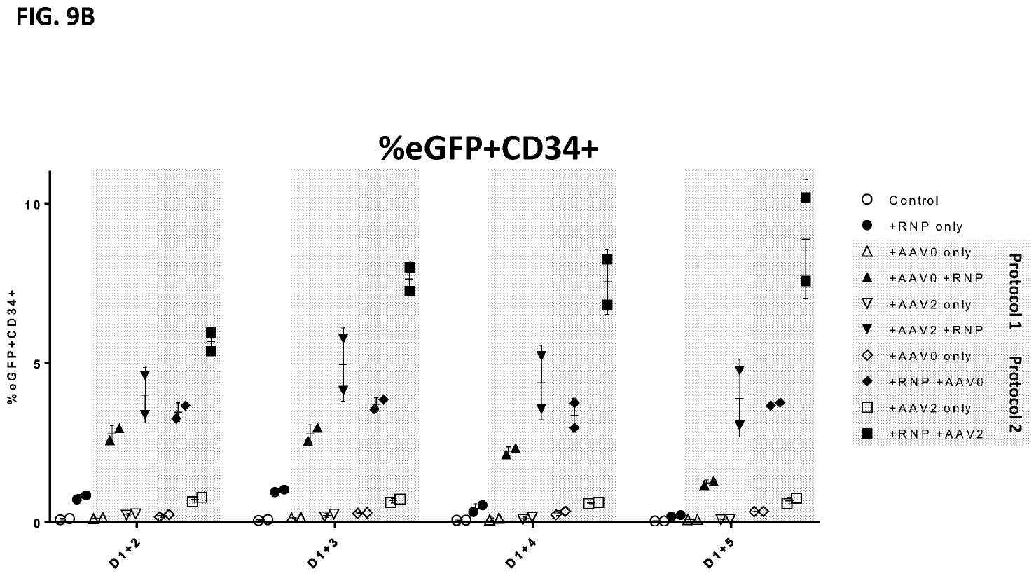

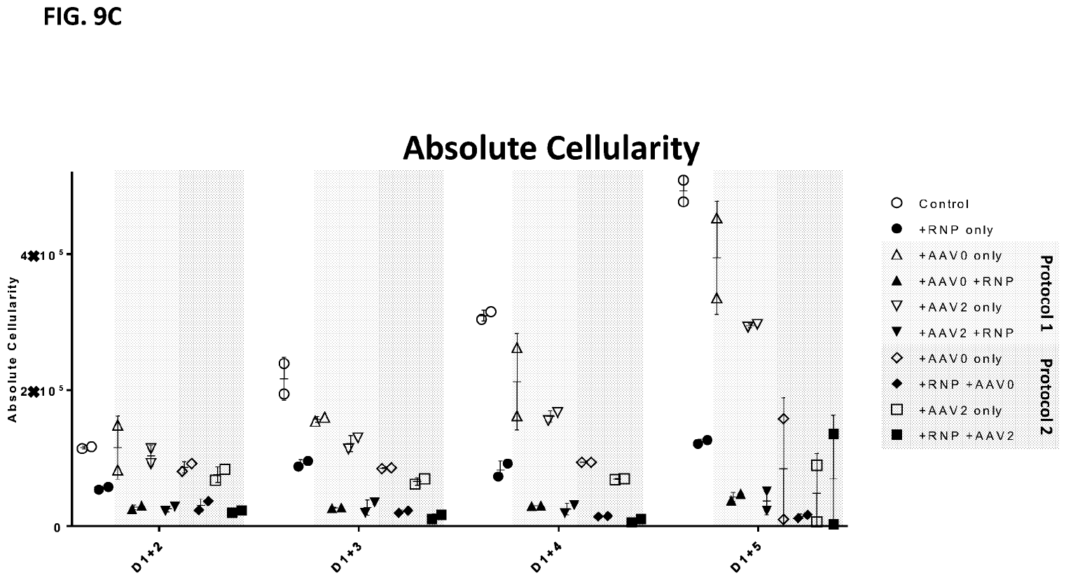

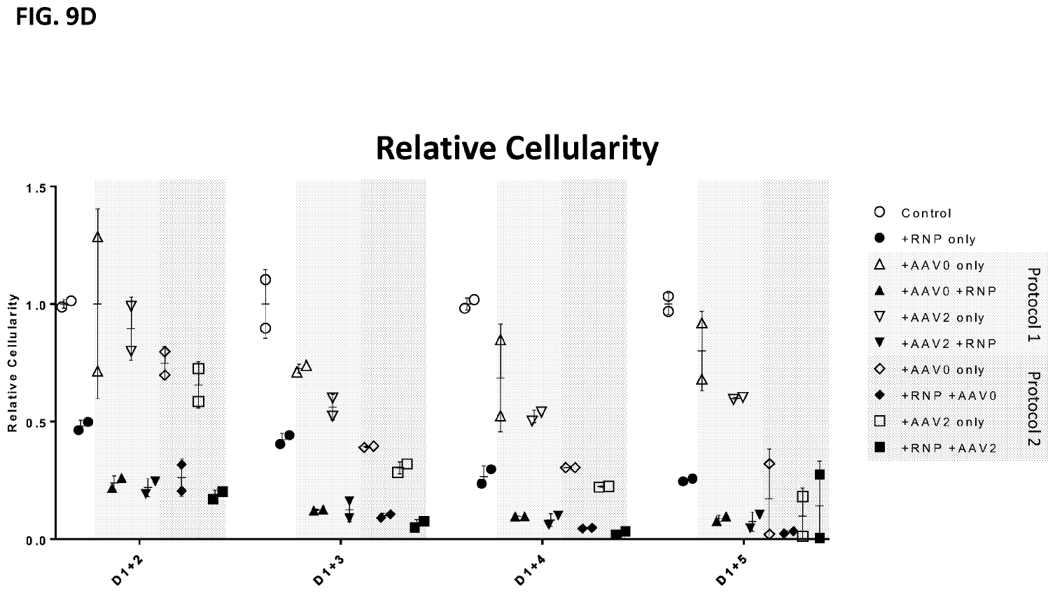

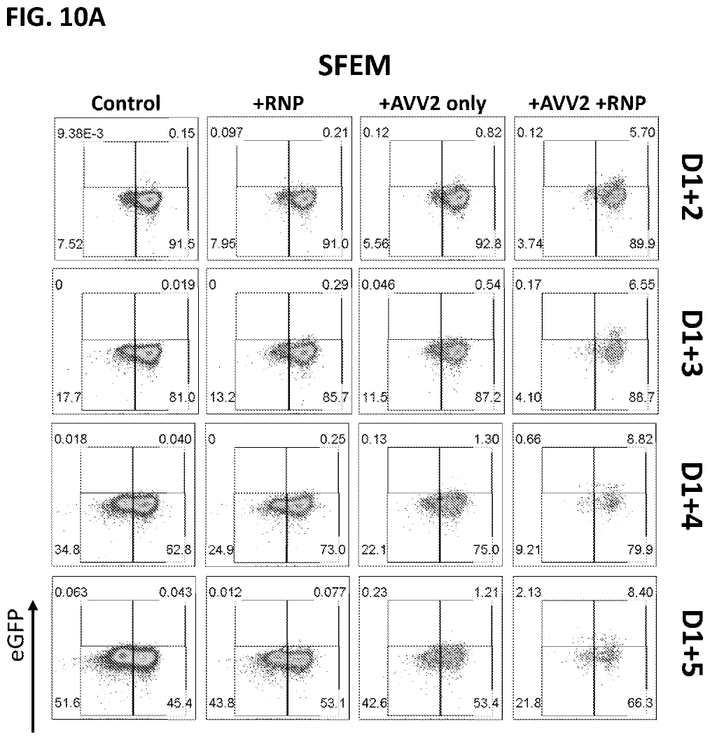

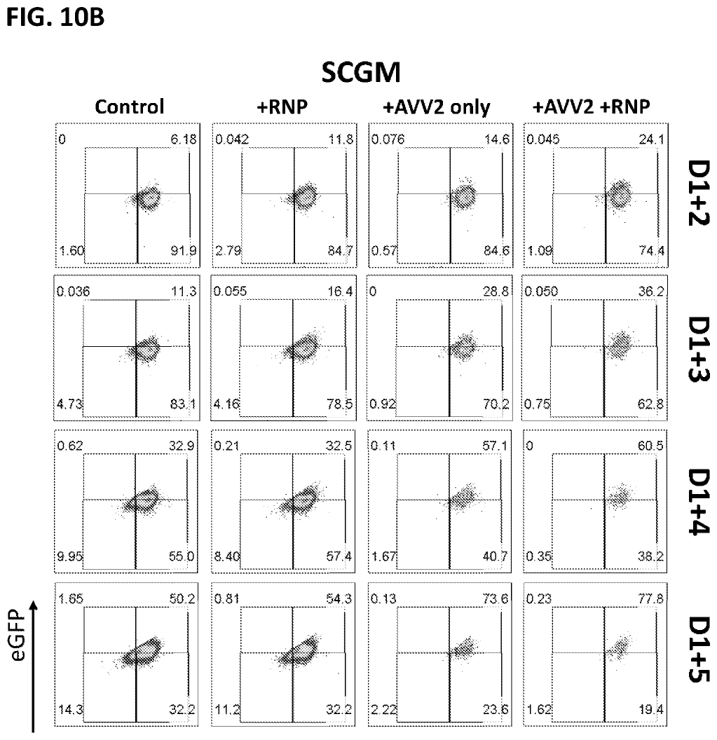

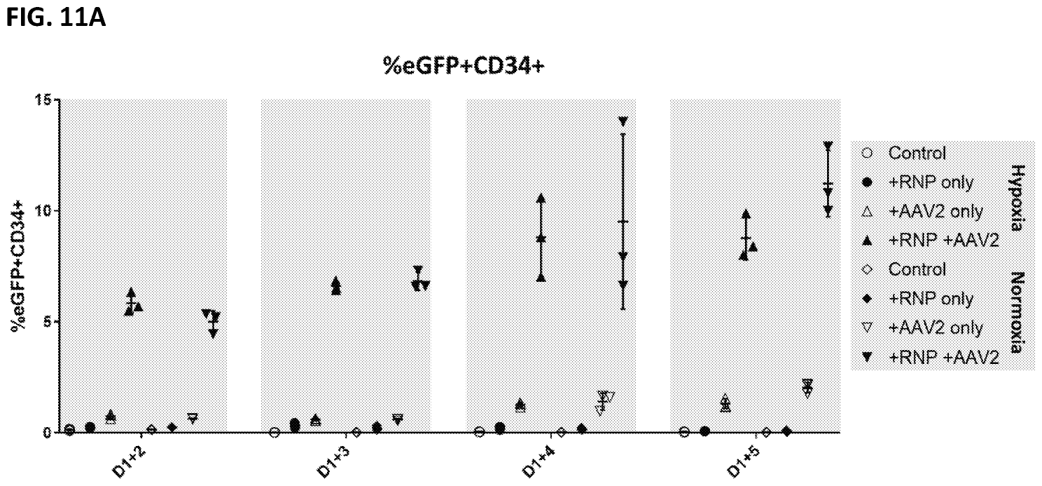

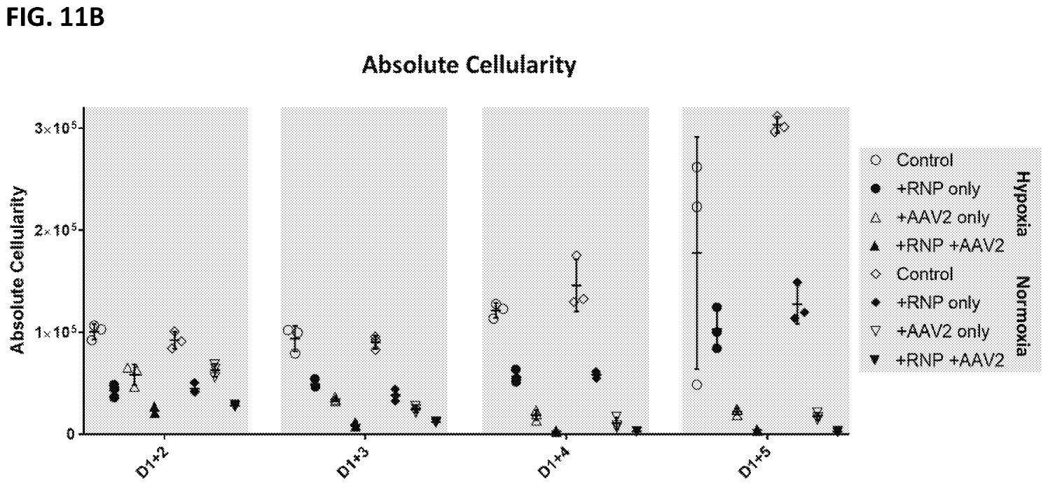

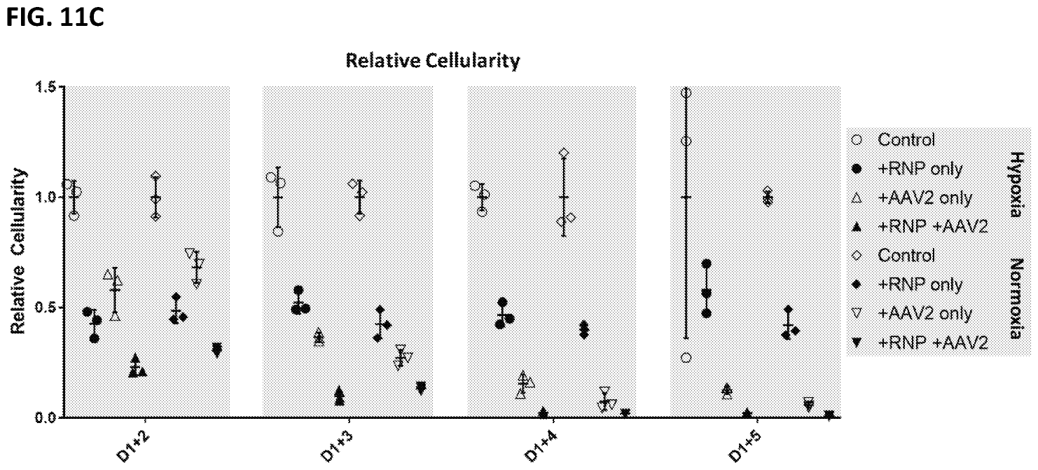

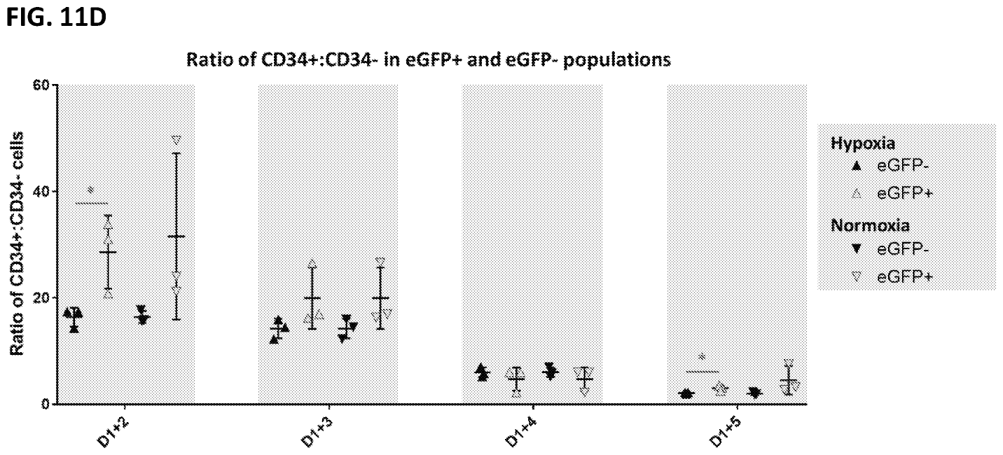

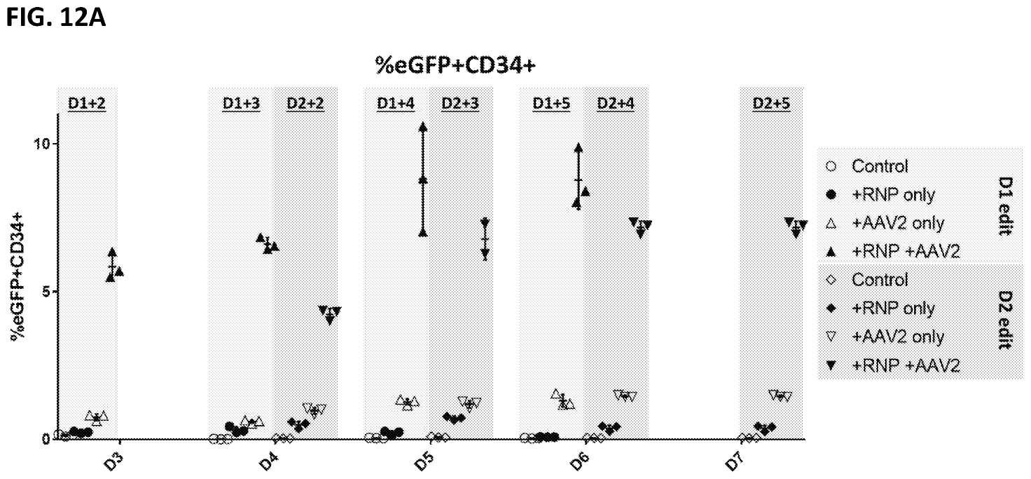

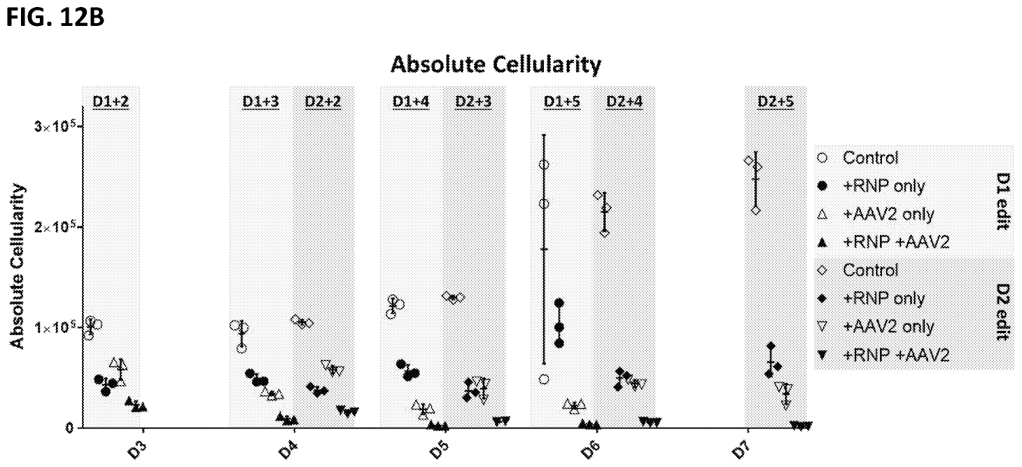

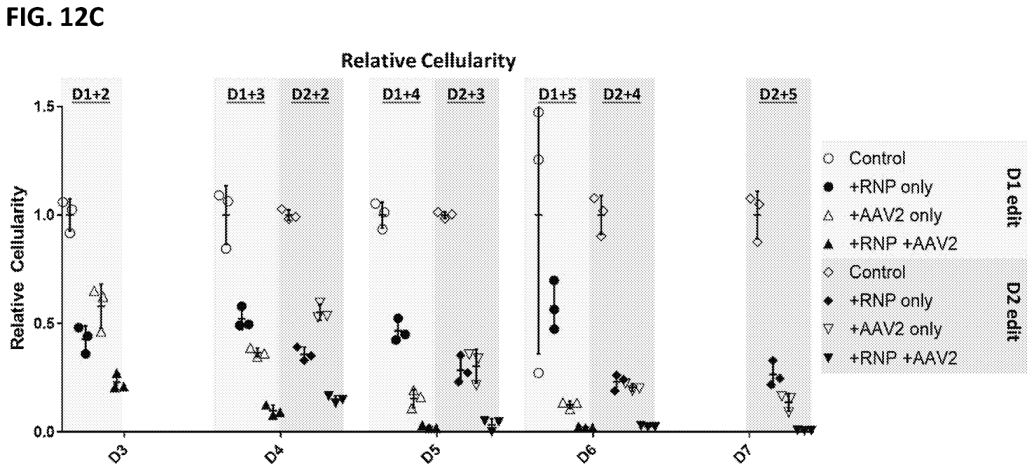

Results are shown in FIGS. 9A, 9B, 9C, 9D, 10A, 10B, 11A, 11B, 11C, 11D, 12A, 12B, and 12C. SCGM media caused a shift in the 500 nm range, causing a shift in negative populations in the eGFP and CD34-BV510 channels. This could explain the lack of a clear population of eGFP⁺ cells. Oxygen levels did not have a significant effect on editing rates. However, hypoxic conditions decreased cell growth, and possibly cell cycling, suggesting that HSCs are more quiescent. LT-HSC populations were retained in hypoxia relative to normoxia. D1-edit showed a slight advantage over D2-edit. Decreasing the amount of time HSCs spend in culture may allow maintenance of engraftment potential. It has previously been shown that HSCs cultured for an extended period of time lose their engraftment potential. While two day pre-stimulation was necessary for HDR-mediated targeted integration, it appears HSCs do not require the extra day for NHEJ-mediated TI.

### Example 5: Testing NHEJ-Mediated TI in CD34 Cells: H1-Edit Vs D1-Edit

This study was carried out to determine in human CD34⁺ cells the feasibility of NHEJ-mediated targeted integration stimulated with and without in vivo donor cleavage. Previously, it was determined that 24 hour (D1) pre-stimulation/rescue culture after thawing is sufficient for NHEJ-mediated targeted integration while maintaining high viability. In this study, it was investigated if a one hour (H1) post-thaw rescue culture was sufficient for efficient and viable NHEJ-mediated TI relative to a 24 hour post-thaw rescue culture.

TABLE 9

| H1-edit | −RNP | +RNP |
|---|---|---|
| Control | Mock Nucleofection | RNP only |
| +AAV 0-cut | AAV only | H2edit |
| D1-edit | | |
| Control | Mock Nucleofection | RNP only |
| +AAV 0-cut | AAV only | D1 edit |

**Experiments done in triplicate

In these experiments, 200,000 CD34⁺ cells/condition (from Stem Cell Technologies) were used. The cells were cultured in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

RNPs included Cas9 (GeneArt V2) and AAVS1 guide RNA (SEQ ID NO: 8) (Synthego) at a 10:25 pmol ratio. scAAV6 0-cut NHEJ vector (SEQ ID NO: 4) (Vector Biolabs) was used at an MOI of 20,000 vg/cell with two hour incubation at 2×10⁶ cells/ml followed by transfer to 0.9 ml fresh media.

Lonza 4D Nucleofector® was used with Primary Cell P3 nucleofector solution (Lonza V4XP-3032) and nucleofection protocol DZ-100 at manufacturer's recommendation.

### Cell Culture

For the H1-edit condition, cells were manipulated as stated below one to two hours after thawing. For the D1-edit condition, cells were manipulated as stated below 24 hours after thawing.

Aliquoted media and cytokines were thawed immediately before use. Thawed cytokines and small molecules were added to SFEM II to create complete media, which was warmed to 37° C. Vials of hCD34⁺ sufficient for 200,000 cells/conditions were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cell s/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube (enough RNP was thawed for subsequent experiments). Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of the nucleofection strips and nucleofection reagent was added up to 10 μl volume. Culture plates were prepared by adding 1 ml complete media to each well of a 24-well plate. Cell supernatants were aspirated or decanted and cells were resuspended in 1/2 volume per sample of Buffer P3+Supplement. Ten μl of the cells in nucleofection reagent were added to each well, and the nucleofector was run according to manufacturer's protocol. After nucleofection 80 μl pre-warmed complete media was immediately added to each well and cells were gently pipetted into each well of the prepared culture plates.

### AAV Infection

AAV were thawed on ice. Thawed cytokines and small molecules were added to SFEM II to create complete media immediately before use, which was warmed to 37° C., during which time AAV dilutions were prepared in low-binding protein tubes. 96-well culture plates were prepared, and 80 μl complete media and corresponding amounts of the AAV dilutions were added to each well. 100 μl of nucleofected cells (with 80 μl rescue media added) were transferred from the 16-well strips to the prepared culture plates, and cells were placed back in a humidified 37° C. hypoxic incubator for two hours. Cells were then collected, during which time culture plates were prepared by adding 0.7 ml complete media in each well of a 24-well plate. Cell supernatants were decanted and cells were resuspended and transferred to the prepared 24-well culture plate. The 96-well culture plates were washed with 200 μl fresh media and vigorous pipetting, and media was transferred to corresponding wells of the 24-well plate.

Cells were then cultured for four days, after which they were collected and pelleted in 1.5 ml tubes. gDNA was collected using the Machery-Nagel genomic DNA extraction kit according to manufacturer's protocol.

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: Denature: 95° C. 00:010:00; 2. Denature: 95° C. 00:00:15; 3. Annealing: 60° C. 00:00:15; 4. Extension: 72° C. 00:02:00 (Repeat steps 2-4 ×35 cycles); 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11); 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15)); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA; 1 μl GC-enhancer; and H₂O up to 20 μl total.

### Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system. The flow cytometry CD34 ex vivo panel included CD34 BV510; CD90 PerCP-Cy5.5; CD38 APC; CD45RA PE; and Ghost Red 780 viability dye.

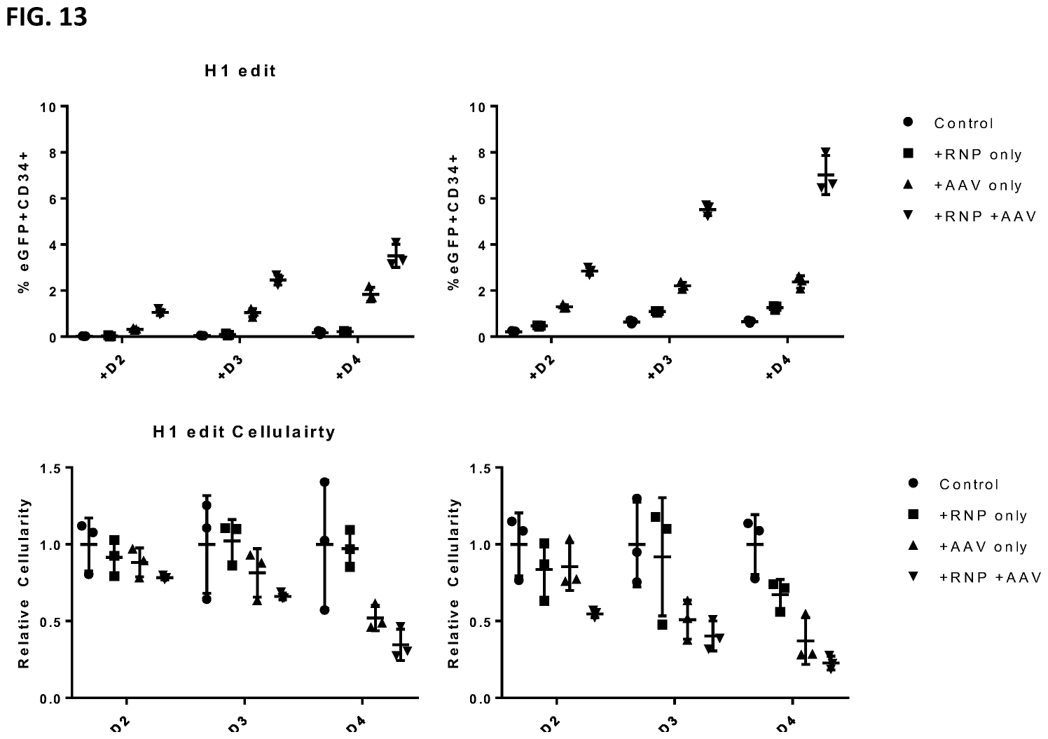

Results are shown in FIG. 13. The cells that were edited one hour post-thaw showed evidence of successful NHEJ-mediated targeted integration, albeit at slightly lower efficiency. However, the relative cellularity and viability was slightly increased when the cells were edited on H1 relative to D1. A protocol where cells are thawed, edited, and injected on the same day could simplify clinical applications of the technology. This would be unique to NHEJ-TI since it does not require a pre-stimulation step as required for HDR-mediated TI.

### Example 6: Testing NHEJ-Mediated TI in CD34 Cells: Nucleofection Protocol (DZ-100 vs CA-137), Cas9 Source (Aldevron SpyFi, GeneArt V2)

This study was carried out to determine in human CD34⁺ cells the feasibility of NHEJ-mediated targeted integration stimulated with and without in vivo donor cleavage. Here, nucleofection protocols were compared, as well as the source of Cas9.

TABLE 6

| DZ-100 | No RNP | +SpyFi RNP | +GeneArt RNP |
|---|---|---|---|
| No virus | Mock | RNP | RNP |
| | Nucleofection | Nucleofection | Nucleofection |
| 2-cut scAAV6 | −RNP | +RNP | +RNP |
| CA-137 | | | |
| No virus | Mock | RNP | RNP |
| | Nucleofection | Nucleofection | Nucleofection |
| 2-cut scAAV6 | −RNP | +RNP | +RNP |

**Experiments done in duplicate

In these experiments, 200,000 CD34⁺ cells/condition (from Stem Cell Technologies) were used. The cells were cultured in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

RNPs included 24 pmols Cas9 (GeneArt V2 vs Aldevron SpyFi) to 120 pmols AAVS1 guide RNA (SEQ ID NO: 8) (Synthego). scAAV6 2-cut NHEJ vector (SEQ ID NO: 5) (Vector Biolabs) was used at an MOI of 20,000 vg/cell with two hour incubation at 2×10⁶ cells/ml followed by transfer to 0.9 ml fresh media.

Lonza 4D Nucleofector® was used with Primary Cell P3 nucleofector solution (Lonza V4XP-3032) and nucleofection protocol DZ-100 or CA-137 at manufacturer's recommendation.

### Cell Culture

Aliquoted media and cytokines were thawed immediately before use. Thawed cytokines and small molecules were added to SFEM II to create complete media, which was warmed to 37° C. Vials of hCD34⁺ sufficient for 200,000 cells/conditions were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cell s/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube (enough RNP was thawed for subsequent experiments). Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of the nucleofection strips and nucleofection reagent was added up to 10 μl volume. Culture plates were prepared by adding 1 ml complete media to each well of a 24-well plate. Cell supernatants were aspirated or decanted and cells were resuspended in 1/2 volume per sample of Buffer P3+Supplement. Ten μl of the cells in nucleofection reagent were added to each well, and the nucleofector was run according to manufacturer's protocol. After nucleofection 80 μl pre-warmed complete media was immediately added to each well and cells were gently pipetted into each well of the prepared culture plates.

### AAV Infection

AAV were thawed on ice. Thawed cytokines and small molecules were added to SFEM II to create complete media immediately before use, which was warmed to 37° C., during which time AAV dilutions were prepared in low-binding protein tubes. 96-well culture plates were prepared, and 80 μl complete media and corresponding amounts of the AAV dilutions were added to each well. 100 μl of nucleofected cells (with 80 μl rescue media added) were transferred from the 16-well strips to the prepared culture plates, and cells were placed back in a humidified 37° C. hypoxic incubator for two hours. Cells were then collected, during which time culture plates were prepared by adding 0.7 ml complete media in each well of a 24-well plate. Cell supernatants were decanted and cells were resuspended and transferred to the prepared 24-well culture plate. The 96-well culture plates were washed with 200 μl fresh media and vigorous pipetting, and media was transferred to corresponding wells of the 24-well plate.

Cells were then cultured for four days, after which they were collected and pelleted in 1.5 ml tubes. gDNA was collected using the Machery-Nagel genomic DNA extraction kit according to manufacturer's protocol.

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: 1. Denature: 95° C. 00:010:00; 2. Denature: 95° C. 00:00:15; 3. Annealing: 60° C. 00:00:15; 4. Extension: 72° C. 00:02:00 (Repeat steps 2-4 ×35 cycles); 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11); 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15)); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA; 1 μl GC-enhancer; and H₂O up to 20 μl total.

### Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system. The flow cytometry CD34 ex vivo panel included CD34 BV510; CD90 PerCP-Cy5.5; CD38 APC; CD45RA PE; and Ghost Red 780 viability dye.

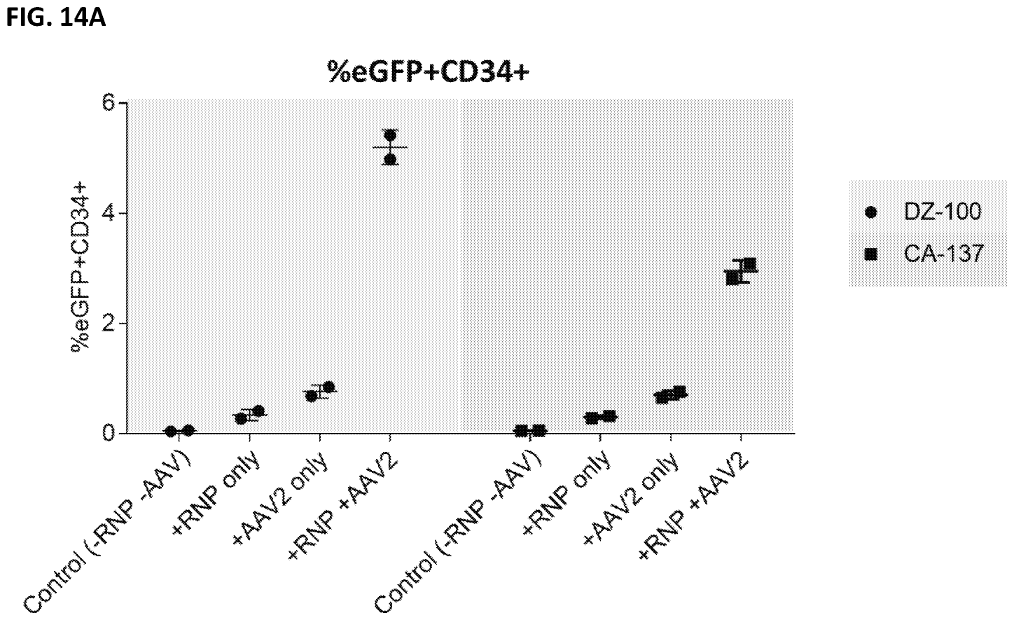

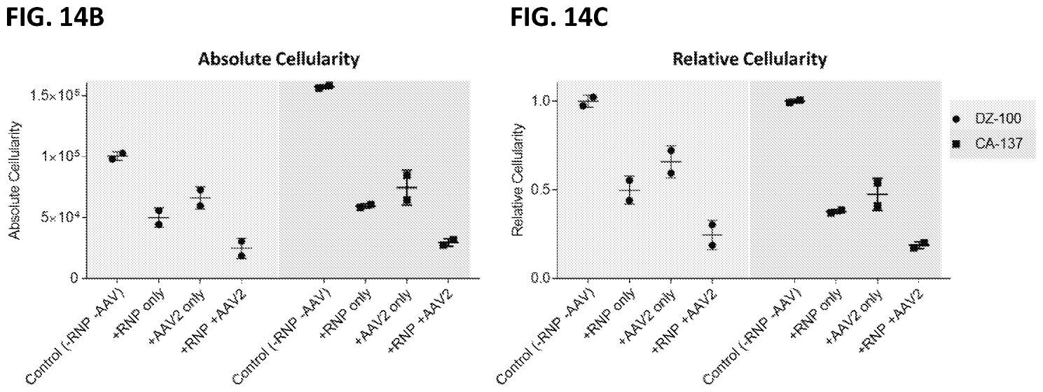

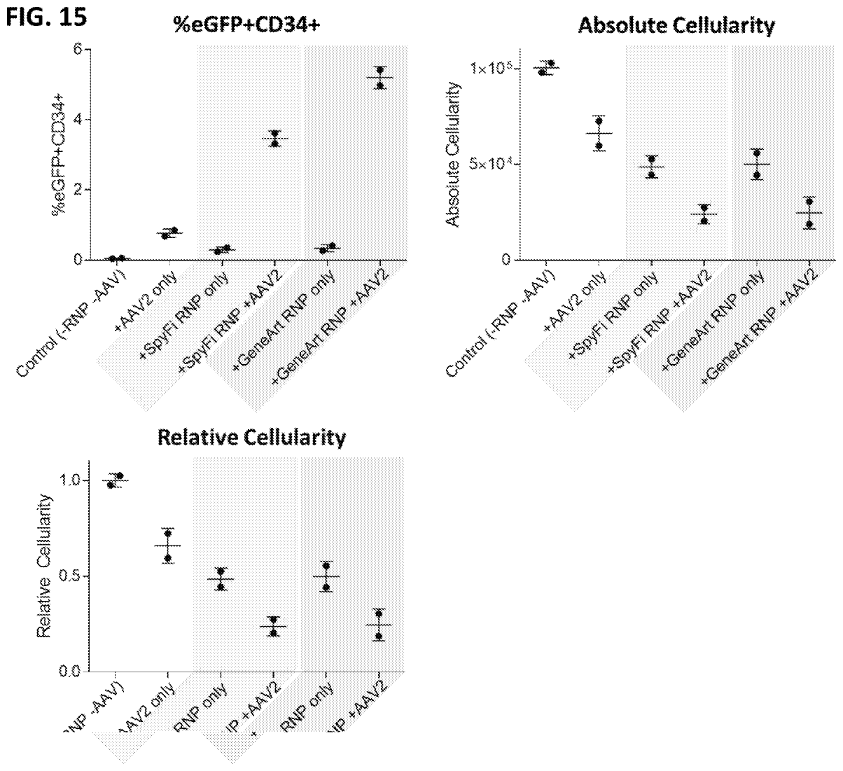

Results are shown in FIGS. 14A, 14B, 14C, and 15. The DZ-100 nucleofection program performed better than the CA-137 nucleofection program. Editing efficiency was significantly higher using DZ-100, though absolute cellularity of the cells was not significantly different. GeneArt V2 Cas9 performed better than Aldevron's SpyFi Cas9. Editing efficiency was significantly higher with GeneArt V2 Cas9, while SpyFi did not improve viability.

### Example 7: Testing NHEJ-Mediated TI in CD34 Cells: RNP and AAV Titration

This study was carried out to determine in human CD34⁺ cells the feasibility of NHEJ-mediated targeted integration stimulated with and without in vivo donor cleavage. In an effort to increase viability while maintaining NHEJ-mediated TI efficiency, RNP levels and AAV levels were titrated.

TABLE 7

| DZ-100 | No RNP | +Lo RNP | +Hi RNP |
|---|---|---|---|
| No virus | Mock Nucleofection | +Lo RNP | +Hi RNP |
| Lo 2-cut scAAV6 | −RNP | +Lo RNP | +Hi RNP |
| Hi 2-cut scAAV6 | −RNP | +Lo RNP | +Hi RNP |

**Experiments done in triplicate

In these experiments, 200,000 CD34⁺ cells/condition (from Stem Cell Technologies) were used. The cells were cultured in SFEM-II (Stem Cell Technologies) with SCF 100 ng/ml; PO 100 ng/ml; FLT3L 100 ng/ml; and IL6 100 ng/ml in hypoxic conditions (5% 02, 5% CO₂, 90% N2).

RNPs included Cas9 (GeneArt V2):AAVS1 guide RNA (SEQ ID NO: 8) (Synthego) at a ratio of 1) Lo: 10:50 pmols; or 2) Hi: 20:100 pmols. scAAV6 2-cut NHEJ vector (SEQ ID NO: 5) (Vector Biolabs) was used at an MOI of 1) Lo: 20,000 vg/cell; or 2) Hi: 60,000 vg/cell, with two hour incubation at 2×10⁶ cells/ml followed by transfer to 0.9 ml fresh media.

Lonza 4D Nucleofector® was used with Primary Cell P3 nucleofector solution (Lonza V4XP-3032) and nucleofection protocol DZ-100 at manufacturer's recommendation.

### Cell Culture

Aliquoted media and cytokines were thawed immediately before use. Thawed cytokines and small molecules were added to SFEM II to create complete media, which was warmed to 37° C. Vials of hCD34⁺ sufficient for 200,000 cells/conditions were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cell s/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube (enough RNP was thawed for subsequent experiments). Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of the nucleofection strips and nucleofection reagent was added up to 10 μl volume. Culture plates were prepared by adding 1 ml complete media to each well of a 24-well plate. Cell supernatants were aspirated or decanted and cells were resuspended in 1/2 volume per sample of Buffer P3+Supplement. Ten μl of the cells in nucleofection reagent were added to each well, and the nucleofector was run according to manufacturer's protocol. After nucleofection 80 μl pre-warmed complete media was immediately added to each well and cells were gently pipetted into each well of the prepared culture plates.

### AAV Infection

AAV were thawed on ice. Thawed cytokines and small molecules were added to SFEM II to create complete media immediately before use, which was warmed to 37° C., during which time AAV dilutions were prepared in low-binding protein tubes. 96-well culture plates were prepared, and 80 μl complete media and corresponding amounts of the AAV dilutions were added to each well. 100 μl of nucleofected cells (with 80 μl rescue media added) were transferred from the 16-well strips to the prepared culture plates, and cells were placed back in a humidified 37° C. hypoxic incubator for two hours. Cells were then collected, during which time culture plates were prepared by adding 0.7 ml complete media in each well of a 24-well plate. Cell supernatants were decanted and cells were resuspended and transferred to the prepared 24-well culture plate. The 96-well culture plates were washed with 200 μl fresh media and vigorous pipetting, and media was transferred to corresponding wells of the 24-well plate.

Cells were then cultured for four days, after which they were collected and pelleted in 1.5 ml tubes. gDNA was collected using the Machery-Nagel genomic DNA extraction kit according to manufacturer's protocol.

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: 1. Denature: 95° C. 00:010:00; 2. Denature: 95° C. 00:00:15; 3. Annealing: 60° C. 00:00:15; 4. Extension: 72° C. 00:02:00 (Repeat steps 2-4 ×35 cycles); 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11) 1 μl; 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15) 1 μl); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA, 1 μl GC-enhancer, and H₂O up to 20 μl total.

### Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system. The flow cytometry CD34 ex vivo panel included CD34 BV510; CD90 PerCP-Cy5.5; CD38 APC; CD45RA PE; and Ghost Red 780 viability dye.

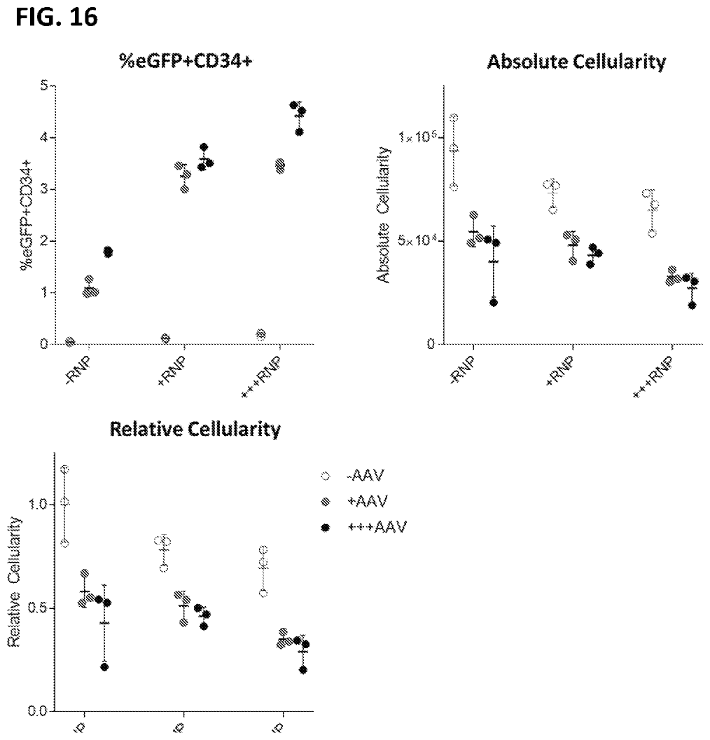

Results are shown in FIG. 16. Decreased RNP levels decreased editing efficiency. Increased AAV levels significantly increased efficiency when used with increased levels of RNP, and did not decrease relative viability.

### Example 8: PCR Analysis of Targeted Donor Integration of 2-Cut Donors

This study was carried out to determine the requirement for Cas9/gRNA RNP in mediating targeted donor integration in human CD34⁺ cells.

In these experiments, 200,000 CD34⁺ cells/condition were used. The cells were cultured in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

RNPs included Cas9 (GeneArt V2):AAVS1 guide RNA (Synthego) at a ratio of 20:50 pmols. Ad5/35 2-cut NHEJ vector (Welgen Inc) was used at an MOI of 5000 vp/cell with two hour incubation at 2×10⁶ cells/ml.

Lonza 4D Nucleofector® was used with Primary Cell P3 nucleofector solution (Lonza V4XP-3032) and nucleofection protocol DZ-100 at manufacturer's recommendation.

RNP nucleofection and viral infection were carried out as previously described. In-out PCR analysis for integration detection was conducted 7 days post nucleofection and Ad5/35 treatment.

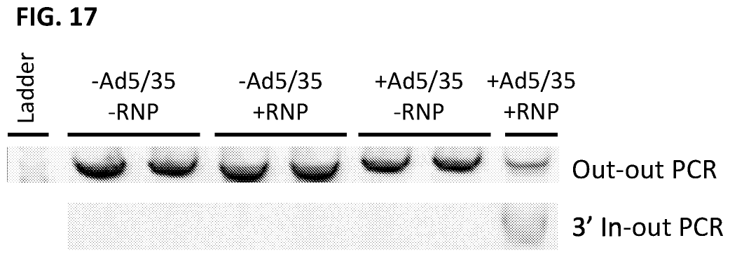

Results are shown in FIG. 17. All samples had the out-out band, but only the sample treated with both Ad5/35 and RNP nucleofection showed integration of the transgene as evidenced by the in-out PCR band.

### Example 9: Testing NHEJ-Mediated TI in CD34 Cells: Culture Conditions—Matrix Proteins

The previous Examples show overall optimized editing conditions for NHEJ-mediated targeted integration in CD34 HSCs. Culture conditions were optimized for HDR, necessitating cell cycling in the HSCs. However, inducing cell proliferation in HSCs generally induces differentiation, depleting the fraction of LT-HSCs. NHEJ-mediated TI does not require the cells to cycle and grow. Here, it was examined whether matrix proteins can help maintain LT-HSC phenotype while maintaining efficient targeted integration. The use of tenascin C, a matrix protein, and DLL1-Fc coating for the induction of NOTCH signaling was examined. Both of these factors have been associated with the bone marrow niche, HSC development, and maintenance.

TABLE 8

| | | | | +TenC |
|---|---|---|---|---|
| | No Coating | +TenC | +DLL1-Fc | +DLL1-Fc |
| No edit | Control | No edit | No edit | No edit |
| +RNP +scAAV6 | Editing | Edit | Edit | Edit |
| 0-cut | control | +TenC | +DLL1-Fc | +Both |

**Experiments done in triplicate

In these experiments, 200,000 CD34⁺ cells/condition (from Stem Cell Technologies) were used. The cells were cultured in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

Culture plates (tissue culture-treated 24-well plates) were prepared as follows. 1. Control plates (low-attachment plates). 2. Tenascin-C: 1 μg/cm² (EMD Millipore); 3. DLL1-Fc: 1 μg/cm² (R&D Systems); 4. Tenascin-C: 1 μg/cm² (EMD Millipore)+DLL1-Fc: 1 μg/cm² (R&D Systems).

RNPs included Cas9 (GeneArt V2):AAVS1 guide RNA (SEQ ID NO: 8) (Synthego) at a ratio of 10:25 pmols. scAAV6 0-cut NHEJ vector (SEQ ID NO: 4) (Vector Biolabs) was used at an MOI of 20,000 vg/cell with two hour incubation at 2×10⁶ cells/ml followed by transfer to 0.9 ml fresh media.

Lonza 4D Nucleofector® was used with Primary Cell P3 nucleofector solution (Lonza V4XP-3032) and nucleofection protocol DZ-100 at manufacturer's recommendation.

### Cell Culture

Cell culture plates coated with Tenascin C were prepared as follows. 100 μg of thawed Tenascin C was diluted in 24 ml cold PBS and sterilized by filtration with a 0.22 μm PVDF membrane, and 0.5 ml/well of the PBS-TenC mixture was added to wells of a 24-well plate. Plates were covered with parafilm and incubated overnight at 4° C. The PBS-TenC mixture was aspirated immediately before use.

Cell culture plates coated with DLL1-Fc were prepared as follows. DLL1-Fc was reconstituted in PBS (manufacturer's protocol) by diluting 50 μg of DLL1-Fc in 12 ml cold PBS, and 0.5 ml/well of the PBS-DLL1-Fc mixture was added to wells of a 24-well plate. Plates were covered with parafilm and incubated overnight at 4° C. The PBS-DLL1-Fc mixture was aspirated immediately before use.

Cell culture plates coated with Tenascin C and DLL1-Fc were prepared as follows. 50 of thawed Tenascin C was diluted in 12 ml cold PBS and sterilized by filtration with a 0.22 PVDF membrane. 50 μg reconstituted DLL1-Fc was added, and 0.5 ml/well of the PBS-TenC-DLL1-Fc mixture was added to wells of a 24-well plate. Plates were covered with parafilm and incubated overnight at 4° C. The PBS-TenC-DLL1-Fc mixture was aspirated immediately before use.

Aliquoted media and cytokines were thawed immediately before use. Thawed cytokines and small molecules were added to SFEM II to create complete media, which was warmed to 37° C. Vials of hCD34⁺ sufficient for 200,000 cells/conditions were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cells/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μl/tube (enough RNP was thawed for subsequent experiments). Cells were collected into 1.5, 15, or 50 ml tubes, and 100 μl (1:100 of total) cells were taken and diluted to 1 ml (1:10 dilution) for measuring viability and cell concentration using a Vi-Cell XR. Cells were spun at 90×g for 10 minutes, during which time corresponding amounts of RNP or mRNA were added into each well of the nucleofection strips and nucleofection reagent was added up to 10 μl volume. Culture plates were prepared by adding 1 ml complete media to each well of a 24-well plate. Cell supernatants were aspirated or decanted and cells were resuspended in 1/2 volume per sample of Buffer P3+Supplement. Ten μl of the cells in nucleofection reagent were added to each well, and the nucleofector was run according to manufacturer's protocol. After nucleofection 80 μl pre-warmed complete media was immediately added to each well and cells were gently pipetted into each well of the prepared culture plates.

### AAV Infection

AAV were thawed on ice. Thawed cytokines and small molecules were added to SFEM II to create complete media immediately before use, which was warmed to 37° C., during which time AAV dilutions were prepared in low-binding protein tubes. 96-well culture plates were prepared, and 80 μl complete media and corresponding amounts of the AAV dilutions were added to each well. 100 μl of nucleofected cells (with 80 μl rescue media added) were transferred from the 16-well strips to the prepared culture plates, and cells were placed back in a humidified 37° C. hypoxic incubator for two hours. Cells were then collected, during which time culture plates were prepared by adding 0.7 ml complete media in each well of a 24-well plate. Cell supernatants were decanted and cells were resuspended and transferred to the prepared 24-well culture plate. The 96-well culture plates were washed with 200 μl fresh media and vigorous pipetting, and media was transferred to corresponding wells of the 24-well plate.

Cells were then cultured for four days, after which they were collected and pelleted in 1.5 ml tubes. gDNA was collected using the Machery-Nagel genomic DNA extraction kit according to manufacturer's protocol.

### TIDE Analysis

Cells were collected into 1.5 ml microcentrifuge tubes. Cells were then rinsed and leftover media was collected with 1 ml PBS. Cells were pelleted by centrifugation for 5 minutes at 350×g, and supernatant was decanted. If total volume was over 1.2 ml, steps 1˜4 were repeated in the same tube. gDNA was extracted using the Qiagen DNeasy® kit (catalog number 69506) according to manufacturer's protocol.

The gDNA samples were PCR amplified using 10 μl AmpliTaq Gold® 360 2× Master Mix, 1 μl Primer 1 AAVS1-outsideF (cggaactctgccctctaacg, SEQ ID NO: 10), 1 μl Primer 2 AAVS1-outsideR (ctgggataccccgaagagtg, SEQ ID NO: 11), 200 ng genomic DNA, 1 μl GC-enhancer, and H₂O up to 20 μl total with the following parameters: 1. Denature: 95° C. 00:010:00; 2. Denature: 95° C. 00:00:15; 3. Annealing: 60° C. 00:00:15; 4. Extension: 72° C. 00:02:00 (Repeat steps 2-4 ×35 cycles); 5. Final Extension 72° C. 00:07:00; 6. Hold 12° C.

The purified PCR product was then sequenced as follows. PCR cleanup was performed using the Qiagen PCR purification kit (catalog number 28106) according to the manufacturer's protocol. The purified PCR products were submitted to Sequetech for Sanger sequencing. Nested primers used for sequencing: AAVS1 TIDE-4R: cctctccatcctcttgctttctttg (SEQ ID NO: 12); and AAVS1 TIDE-4F: aactgcttctcctcttgggaagt (SEQ ID NO: 13).

INDEL analysis was carried out for CRISPR/Cas9 cutting efficiency using TSUNAMI Batch TIDE Analysis (webpage at 54.234.74.37/home/ or 54.158.189.0/home/). The required data files were uploaded to begin analysis: CSV List of Files—a “.csv” file containing four columns with no header (List of sample names—used for labeling in output files; Expected gRNA sequence—a 20 nt (5′-3′) DNA character string representing the expected gRNA sequence immediately upstream of the PAM sequence (PAM not included); Name of test sample; Name of control sample); Sequencing Files (.abl or .scf file). The following parameters were entered for analysis: Left boundary: Default is 100 bp; Right boundary: Default set at break site—10 bp; Decomposition Window: Determines the sequence segment used for decomposition (The default setting is the largest window possible for the uploaded sequences); Indel Size Range: Set the maximum size of indels to be modeled. The default value is 10; P-Value Threshold: Default is p<0.001. Results were then generated and quality measures in output were assessed and parameters adjusted if necessary (Average aberrant sequence signal before the break site <10% (both control and test sample); R2>0.9 for the decomposition result). Reference: Brinkman et al, Nucleic Acids Res. 2014 Dec. 16; 42(22):e168. doi: 10.1093/nar/gku936.

### In-Out PCR for Integration Detection

The same gDNA extracted for TIDE analysis was used for in-out PCR for integration detection. gDNA samples were PCR amplified using 10 μl AmpliTaq Gold® 360 2× Master Mix, one of the following primer combinations: 5′ correct orientation AB (1 μl Primer A: AAVS1-outsideF (cggaactctgccctctaacg, SEQ ID NO: 10); 1 μl Primer B: IntAmp1 (GATGGGGGTGTTCTGCTGG, SEQ ID NO: 14)); 3′ correct orientation CV (1 μl Primer V: AAVS1-outsideR (ctgggataccccgaagagtg, SEQ ID NO: 11) 1 μl; 1 μl Primer C: IntAmp2 (CGTAAACGGCCACAAGTTCA, SEQ ID NO: 15)); 5′ reverse orientation AC (1 μl Primer A: AAVS1-outsideF (cggaactctgccctctaacg, SEQ ID NO: 10); 1 μl Primer C: IntAmp2 (CGTAAACGGCCACAAGTTCA, SEQ ID NO: 15) 1 μl); 3′ reverse orientation BV (1 μl Primer V: AAVS1-outsideR (ctgggataccccgaagagtg, SEQ ID NO: 11); 1 μl Primer B: IntAmp1 (GATGGGGGTGTTCTGCTGG, SEQ ID NO: 14)); 100 ng genomic DNA, 1 μl GC-enhancer, and H₂O up to 20 μl total.

### Flow Cytometry

Cells were collected from cell culture plates into 5 ml FACS tubes, and up to 4 ml of FACS buffer was added. Cells were spun at 350×g for 5 minutes and supernatant was decanted. Viability dye and/or conjugated antibodies for cell surface antigens was added and cells were incubated for 20 minutes at RT. Up to 4 ml of FACS buffer was added and cells were spun at 350×g for 5 minutes. Supernatant was decanted and total volume was brought up to 200 μl, and cells were transferred to U-bottom 96-well plates and run using Attune high throughput system. The flow cytometry CD34 ex vivo panel included CD34 BV510; CD90 PerCP-Cy5.5; CD38 APC; CD45RA PE; and Ghost Red 780 viability dye.

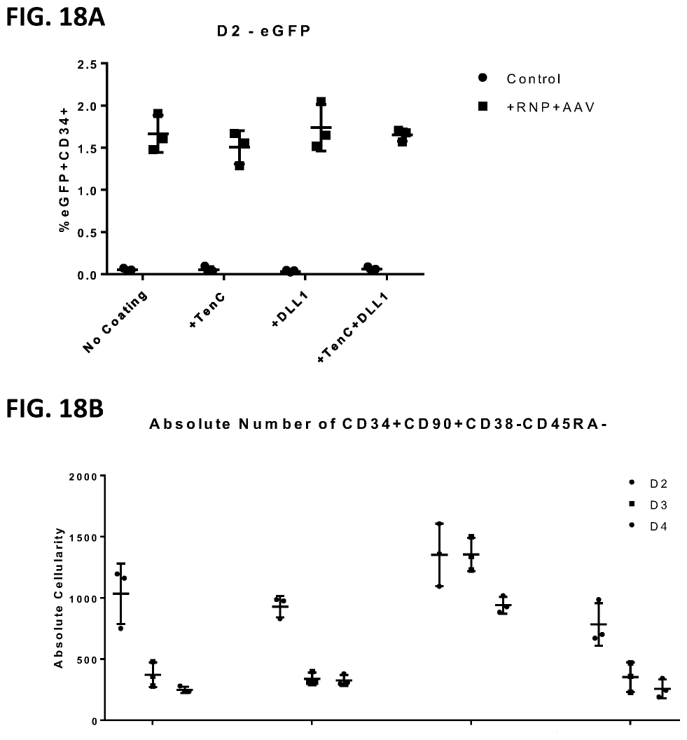

Results are shown in FIGS. 18A, and 18B. Culturing the cells on matrix proteins did not increase viability and absolute cellularity of edited cells relative to control cells. Interestingly, cells cultured on DLL1-Fc had sustained cell numbers with an LT-HSC phenotype (CD34⁺CD91⁺CD45RA⁻CD38″). The effect of Tenascin C negated the effect of DLL1-Fc. Neither DLL1-Fc nor Tenascin C had an effect on NHEJ-mediated targeted integration efficiency.

### Example 10: NHEJ-TI Engraftment Study 2

This study was carried out to determine in human CD34⁺ cells the feasibility of NHEJ-mediated targeted integration stimulated with and without in vivo donor cleavage. Here, the engraftment potential of CD34⁺ HSCs modified by NHEJ-TI, with and without intracellular donor cleavage before injection, was examined.

A total of 20 mice were used in this study for the following conditions. Culture Control: three mice; RNP only: three mice; scAAV6 0-cut only: three mice; scAAV6 2-cut only mice: three mice; RNP+scAAV6 0-cut: four mice; and RNP+scAAV6 2-cut: four mice.

500,000 CD34⁺ cells/mouse (from Stem Cell Technologies) were used, culture in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

RNPs included Cas9 (GeneArt V2):AAVS1 guide RNA (SEQ ID NO: 8) (Synthego) at a ratio of 50:250 pmols. scAAV6 0-cut NHEJ vector (SEQ ID NO: 4) (Vector Biolabs) or scAAV6 2-cut NHEJ vector (SEQ ID NO: 5) (Vector Biolabs) were used at an MOI of 60,000 vg/cell with two hour incubation at 2×10⁶ cells/ml.

Lonza 4D Nucleofector® was used with Primary Cell P3 nucleofector solution (Lonza V4XP-3032) and nucleofection protocol DZ-100 at manufacturer's recommendation.

The experimental timeline was as follows. Day 0 (D0)—cells were thawed: 10 million mobilized CD34⁺ HSCs (Stem Cell Technologies). Day 1 (D1)—Cell were manipulated: nucleofection of RNP and scAAV6 infection. Day 2 (D2)—mice were injected: mice irradiated @ 200 cGy in the morning followed by injection in the afternoon. Week 6—perform interim bleed. Week 9—perform interim bleed. Week 13—perform interim bleed. Week 16—end-point analysis.

### Cell Culture

Aliquoted media and cytokines were thawed immediately before use. Thawed cytokines and small molecules were added to SFEM II to create complete media, which was warmed to 37° C. Vials of hCD34⁺ sufficient for 500,000 cells/mouse were thawed, and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 200,000 cells/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μL/tube (enough RNP was thawed for subsequent experiments). Cells were collected into 50 mL tubes and 100 μL (1:100 of total) cells were taken and diluted to 1 mL (1:10 dilution) for measuring viability and cell concentration using a Vi-Cell XR. Cells were spun at 90×g for 10 minutes, during which time corresponding amounts of RNP were added into each nucleofection cuvette and nucleofection reagent was added up to 50 volume. Culture flasks were prepared by adding 5 mL complete media to individual T25 flasks for each condition. Cell supernatants were aspirated or decanted and cells were resuspended in 1/2 volume per sample of Buffer P3+Supplement. Fifty μL of the cells in nucleofection reagent were added to each cuvette, and the nucleofector was run according to manufacturer's protocol. After nucleofection 500 μL pre-warmed complete media was immediately added to each cuvette and cells were gently pipetted into the prepared culture flasks.

### AAV Infection

AAV were thawed on ice. Thawed cytokines and small molecules were added to SFEM II to create complete media immediately before use, which was warmed to 37° C., during which time AAV dilutions were prepared in low-binding protein tubes. Culture flasks were prepared by adding 750 μl or 1 ml complete media and corresponding amounts of the AAV dilutions to T75 flasks. 600 μl of nucleofected cells (with 500 μl rescue media added) were transferred from the nucleofection flasks to the prepared AAV infection culture flasks, and cells were placed back in a humidified 37° C. hypoxic incubator for six hours. Cells were then transferred to 15 ml conical tubes and spun at 90×g for 10 minutes, supernatant was aspirated, and cells were resuspended in 7.5 ml or 10 ml and transferred to T75 flasks for continued culturing overnight, reserving an aliquot of cells to continue culture for editing analysis.

Cells were collected and pelleted in 15 ml conical tubes, supernatant was aspirated, and cells were resuspended in 165 μl or 220 μl injection buffer (PBS+0.5% BSA). A 10 μl aliquot of cells was diluted 1:100 and counted using a Vi-Cell. The remaining cells were kept on ice until the time of injection. The level of cell loss from thawing the cells, during manipulation, to preparing for injection was greater than anticipated and each mouse only received up to 200,000 cells.

The editing efficiency of the cells were as follows:

D2 eGFP levels: Culture Control: 0.31% eGFP⁺; RNP only: 0.21% eGFP⁺; scAAV6 0-cut only: 0.28% eGFP⁺; scAAV6 2-cut only: 1.29% eGFP⁺; RNP+scAAV6 0-cut: 1.10% eGFP⁺; RNP+scAAV6 2-cut: 2.93% eGFP⁺.

D3 eGFP levels: Culture Control: 0.064% eGFP⁺; RNP only: 0.17% eGFP⁺; scAAV6 0-cut only: 0.32% eGFP⁺; scAAV6 2-cut only: 0.71% eGFP⁺; RNP+scAAV6 0-cut: 1.64% eGFP⁺; RNP+scAAV6 2-cut: 4.14% eGFP⁺.

D4 eGFP levels: Culture Control: 0.24% eGFP⁺; RNP only: 0.38% eGFP⁺; scAAV6 0-cut only: 0.35% eGFP⁺; scAAV6 2-cut only: 0.53% eGFP⁺; RNP+scAAV6 0-cut: 1.67% eGFP⁺; RNP+scAAV6 2-cut: 4.49% eGFP⁺.

These data further demonstrate the improved editing efficiency when employing donor cleavage.

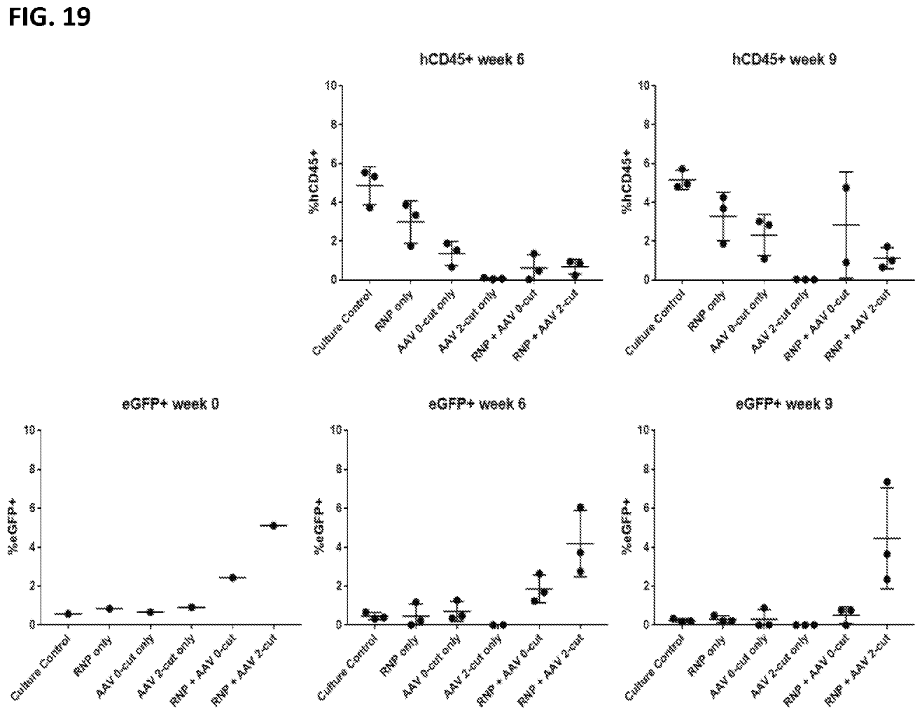

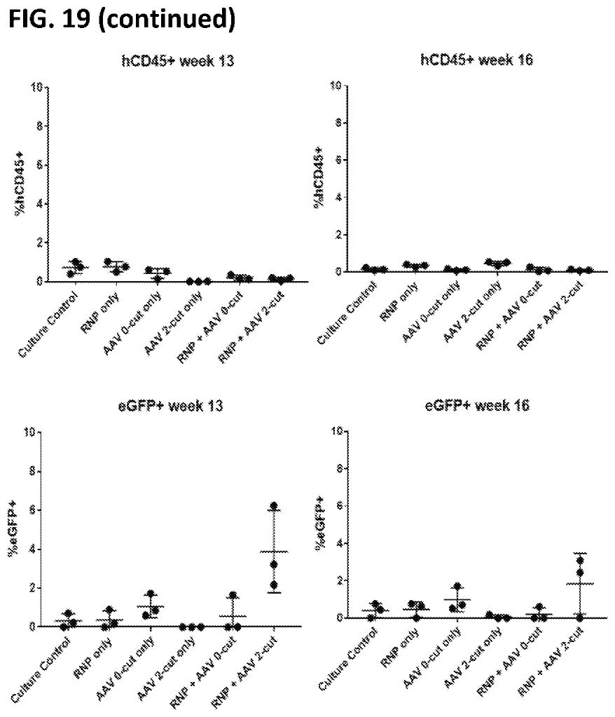

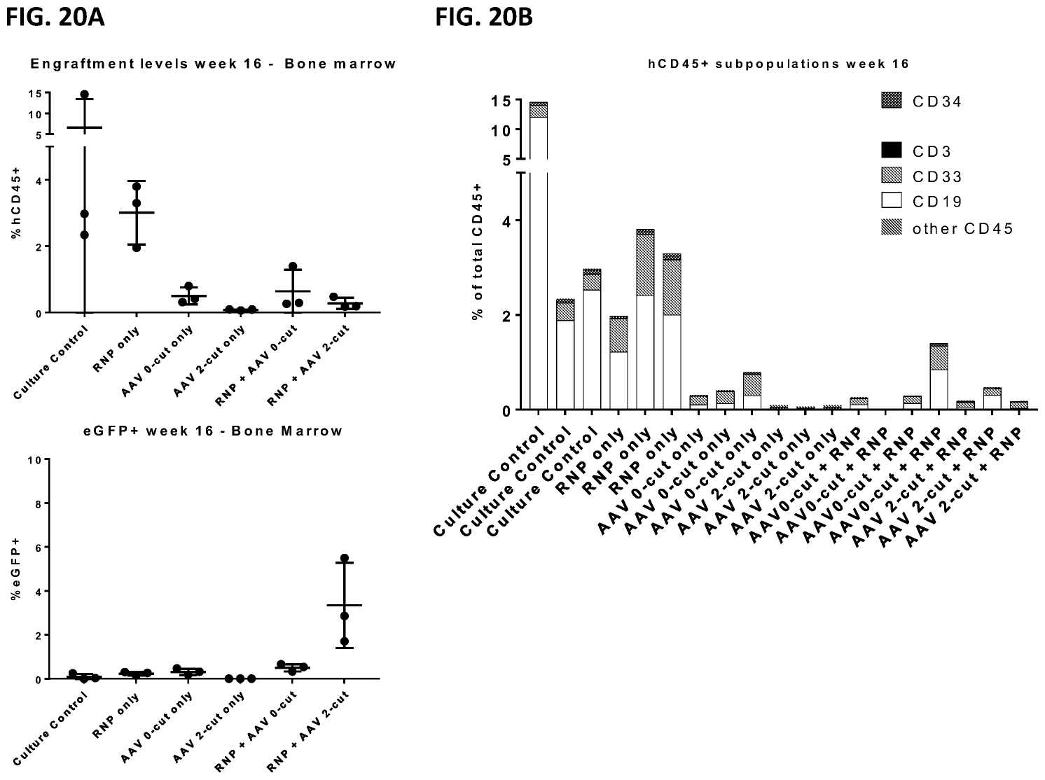

Results are shown in FIG. 19 for hCD45⁺ cells (top panels) and GFP⁺ cells (bottom panels) at weeks 0, 6, 9, 13, and 16 from peripheral blood of mice injected at D2 with human CD34⁺ cells edited at D1 by NHEJ-mediated targeted integration of scAAV6 0-cut or scAAV6 2-cut, demonstrating the persistence of GFP⁺ cells in the mice injected with cells treated with RNP+scAAV6 2-cut through week 16 following injection and suggesting the edited cells were long-term engrafting HSCs. FIG. 20A shows results for hCD45⁺ cells (top panel) and GFP⁺ cells (bottom panel) at 16 weeks from bone marrow of the mice, further demonstrating the persistence of GFP⁺ cells in the mice injected with cells treated with RNP+scAAV6 2-cut at this timepoint. FIG. 20B shows results for the relative amount of hCD34⁺, hCD3⁺, hCD33⁺, hCD19⁺, and other hCD45⁺ cells as a percent of total CD45⁺ cells from bone marrow of the mice at 16 weeks, demonstrating that the largest subpopulation of hCD45⁺ cells in mice injected with cells treated with RNP+scAAV6 2-cut at this timepoint were CD33⁺. The results are surprising and differ from what is generally seen with HDR-mediated targeted integration in HSPCs, where the transgenic fraction often declines precipitously.

### Example 11: NHEJ-TI Engraftment Study 3

In this study, the NHEJ-mediated targeted integration protocol was further optimized. The engraftment potential of CD34⁺ HSCs modified by NHEJ-TI was evaluated, accounting for cell loss during manipulation. The engraftment potential of CD34⁺ HSCs cultured on DLL1-Fc and thaw-edit-inject conditions was also evaluated.

Thirty-six mice were used in this experiment: 1) PBS control: three mice; 2) culture control: three mice; 3) RNP only: three mice; 4) scAAV6 0-cut only: three mice; 5) scAAV6 2-cut only: three mice; 6) RNP+scAAV6 0-cut: four mice; 7) RNP+scAAV6 2-cut: four mice; 8) RNP+scAAV6 2-cut on DLL1 coated plates: four mice; 9) Fresh Thaw: three mice; 10) RNP+scAAV6 2-cut (D0 edit—thaw—inject): four mice.

One million CD34⁺ cells/mouse (from Stem Cell Technologies) were used for the D1+1 condition. 1.6 million CD34⁺ cells/mouse were thawed to account for cell loss. 500,000 CD34⁺ cells/mouse were used for the Fresh Thaw and Culture control conditions. Cells were cultured in SFEM-II (Stem Cell Technologies) with SCF 100 ng/ml; TPO 100 ng/ml; FLT3L 100 ng/ml; and IL6 100 ng/ml in hypoxic conditions (5% 02, 5% CO₂, 90% N2).

RNPs included Cas9 (Feldan):AAVS1 guide RNA (SEQ ID NO: 8) (Synthego) at a ratio of 50:250 pmols. scAAV6 0-cut NHEJ vector (SEQ ID NO: 4) (Vector Biolabs) and scAAV6 2-cut NHEJ vector (SEQ ID NO: 5) (Vector Biolabs) were used at an MOI of 50,000 vg/cell with two hour incubation at 2×10⁶ cells/ml.

Lonza 4D Nucleofector® was used with Primary Cell P3 nucleofector solution (Lonza V4XP-3032) and nucleofection protocol DZ-100 or CA-137 at manufacturer's recommendation.

The experimental timeline used in the study was as follows. Day 0 (D0): cells were thawed; D1+1 injection: 40 million CD34+ cells (GCSF-mobilized). Day 1 (D1): D1+1 cells were manipulated; nucleofection of RNP and scAAV6 infection. Day 2 (D2): mice were injected; Morning—mice irradiated @ 200 cGy; D0 Thaw-Edit-Inject condition: cells were manipulated; Afternoon—D1+1 condition: mice were injected; D0 condition: mice were injected. Weeks 8, 10, 12, and 14: Interim bleed. Week 16: End-point analysis.

### Cell Culture

Aliquoted media and cytokines were thawed immediately before use. Thawed cytokines and small molecules were added to SFEM II to create complete media, which was warmed to 37° C. Vials of hCD34⁺ sufficient for 1,600,000 cells/mouse were thawed (accounting for cell loss), and cells were transferred to 15 ml conical tubes and total volume was brought up to 15 ml with CD34⁺ culture media. Cells were spun at 90×g for 9 minutes, and supernatant was aspirated. Cells were resuspended in 1 ml CD34⁺ culture media, and total volume was brought up to at least 20 ml in low-binding T75 flasks (at most, 250,000 cells/ml).

### RNP Nucleofection

Lonza 4D Nucleofector® was used with the appropriate program for each well. Nucleofection Buffer P3 was pre-mixed with supplement, and Cas9:gRNA RNPs were prepared as previously described and aliquoted at 10 μL/tube (enough RNP was thawed for subsequent experiments). Cells were collected into 50 mL tubes and 100 μL (1:100 of total) cells were taken and diluted to 1 mL (1:10 dilution) for measuring viability and cell concentration using a Vi-Cell XR. Cells were spun at 90×g for 10 minutes, during which time corresponding amounts of RNP were added into each nucleofection cuvette and nucleofection reagent was added up to 50 volume. Culture flasks were prepared by adding 5 mL complete media to individual T25 flasks for each condition. Cell supernatants were aspirated or decanted and cells were resuspended in 1/2 volume per sample of Buffer P3+Supplement. Fifty μL of the cells in nucleofection reagent were added to each cuvette, and the nucleofector was run according to manufacturer's protocol. After nucleofection 500 μL pre-warmed complete media was immediately added to each cuvette and cells were gently pipetted into the prepared culture flasks.

### AAV Infection

AAV were thawed on ice. Thawed cytokines and small molecules were added to SFEM II to create complete media immediately before use, which was warmed to 37° C., during which time AAV dilutions were prepared in low-binding protein tubes. Culture flasks were prepared by adding 750 μl or 1 ml complete media and corresponding amounts of the AAV dilutions to T75 flasks. 600 μl of nucleofected cells (with 500 μl rescue media added) were transferred from the nucleofection flasks to the prepared AAV infection culture flasks, and cells were placed back in a humidified 37° C. hypoxic incubator for six hours. Cells were then transferred to 15 ml conical tubes and spun at 90×g for 10 minutes, supernatant was aspirated, and cells were resuspended in 7.5 ml or 10 ml media and transferred to T75 flasks for continued culturing overnight, reserving an aliquot of cells to continue culture for editing analysis.

Cells were collected and pelleted in 15 ml conical tubes, supernatant was aspirated, and cells were resuspended in 165 μl or 220 μl injection buffer (PBS+0.5% BSA). A 10 μl aliquot of cells was diluted 1:100 and counted using a Vi-Cell. The remaining cells were kept on ice until the time of injection.

DLL1-Fc condition specific protocol: 1) T75 flasks were coated with DLL1-Fc as described previously in Example 9; 2) Cells were thawed and cultured on DLL1-Fc coated plates overnight; 3) To detach the cells for manipulation, the supernatant was collected, and the plate was washed and incubated with 1 mM EDTA in PBS for 5 minutes twice; 4) Cells were edited as described above; 5) After RNP nucleofection, AAV infection was conducted in new DLL1-Fc coated T75 flask; 6) Cells were cultured overnight; 7) Cells were prepared for injection as described above, but again, plates were washed and incubated in 1 mM EDTA in PBS for 5 minutes twice.

Thaw-Edit-Inject condition specific protocol: 1) Cells were thawed on day of injection; 2) Cells were rested for one hour in complete media in low attachment T75 flasks; 3) Cells were manipulated as described above; 4) After two hours of AAV infection, cells were collected instead of diluted, and prepared for injection.

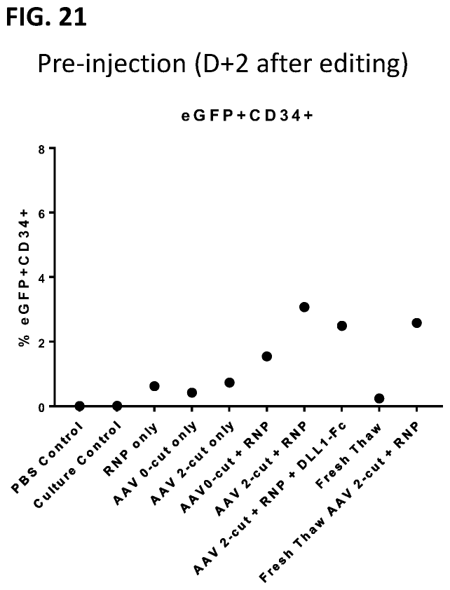

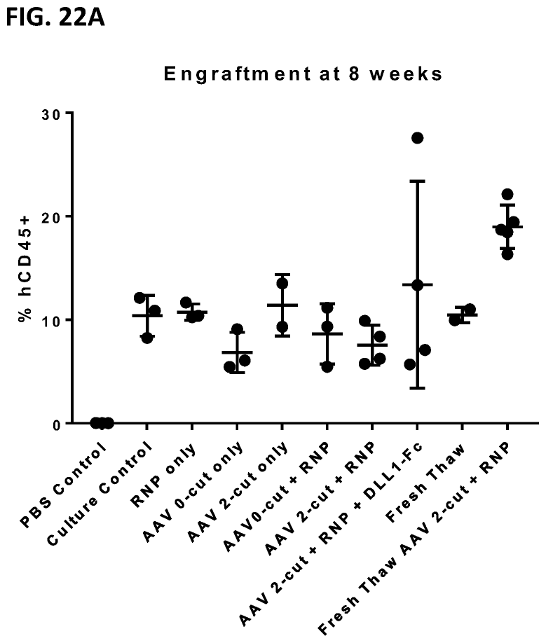

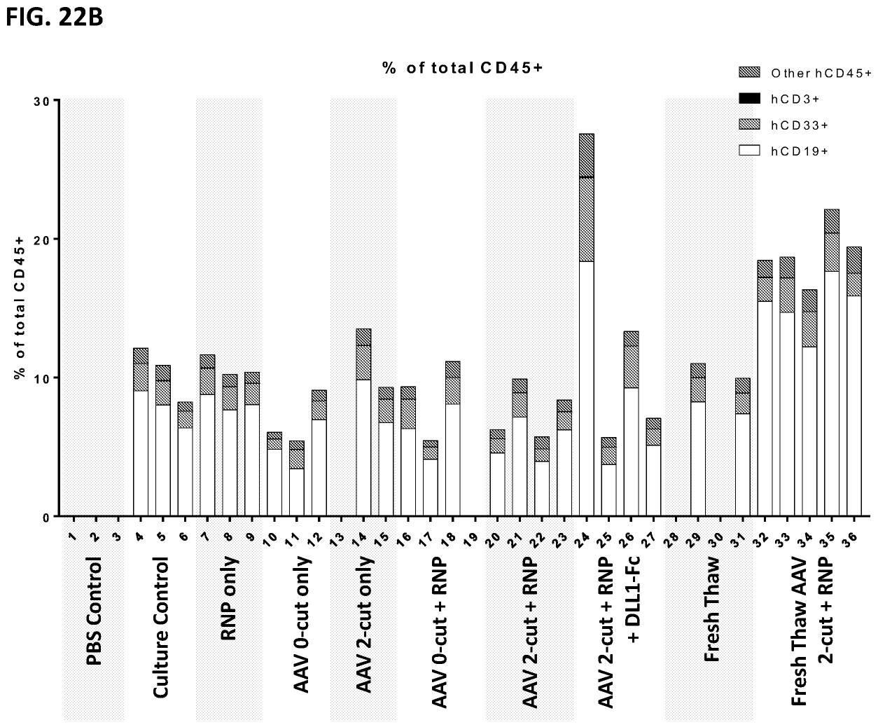

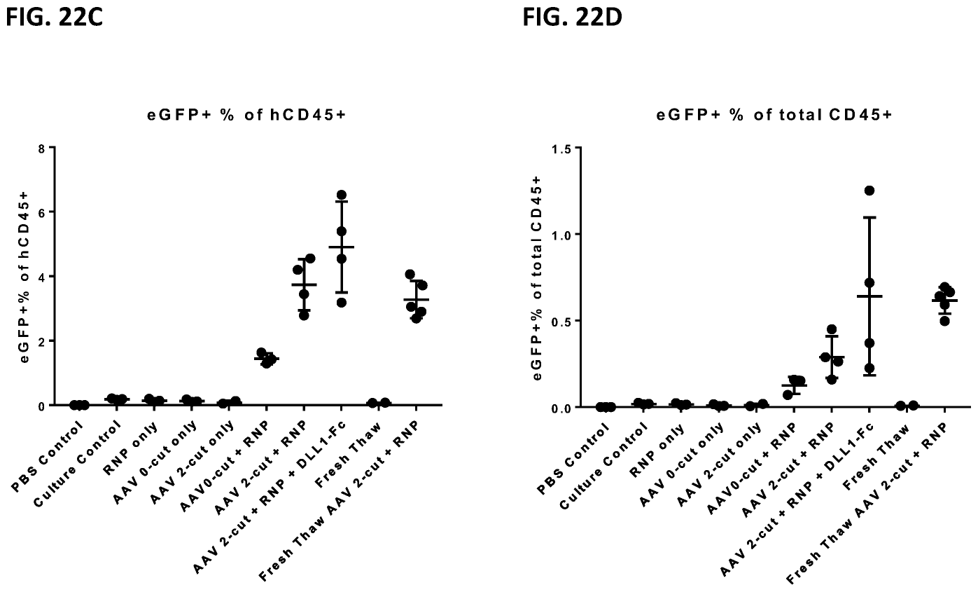

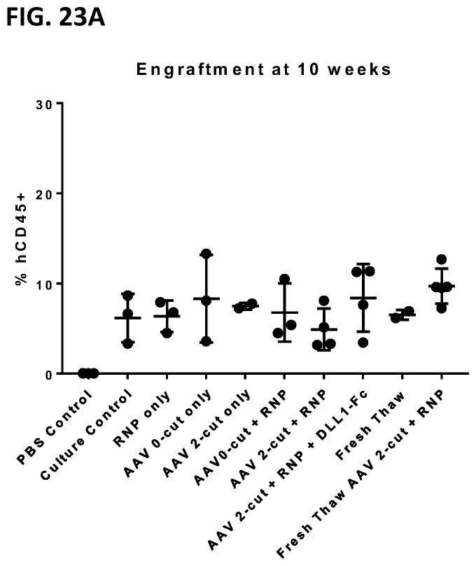

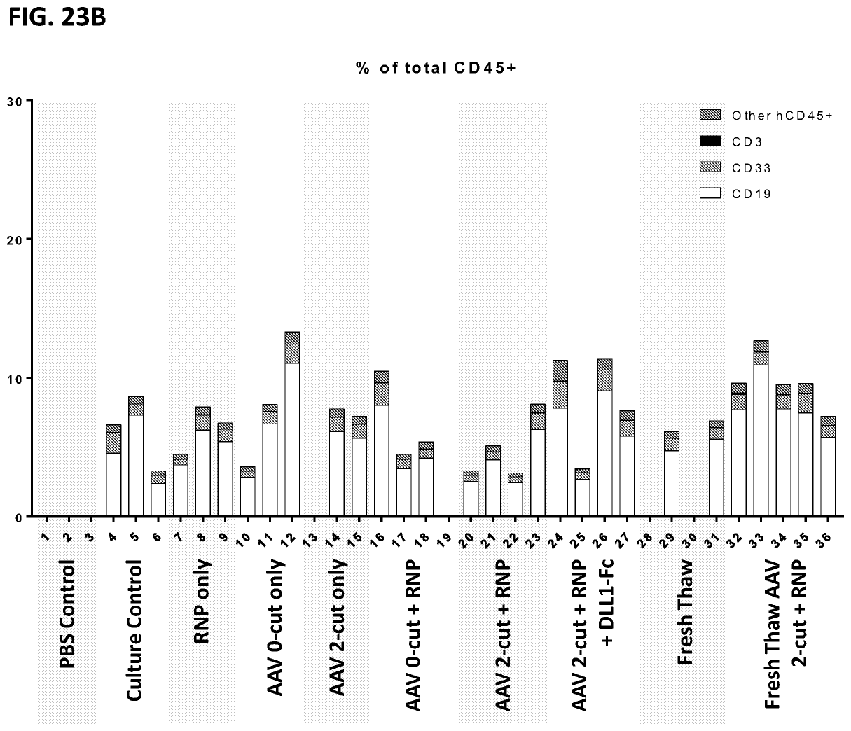

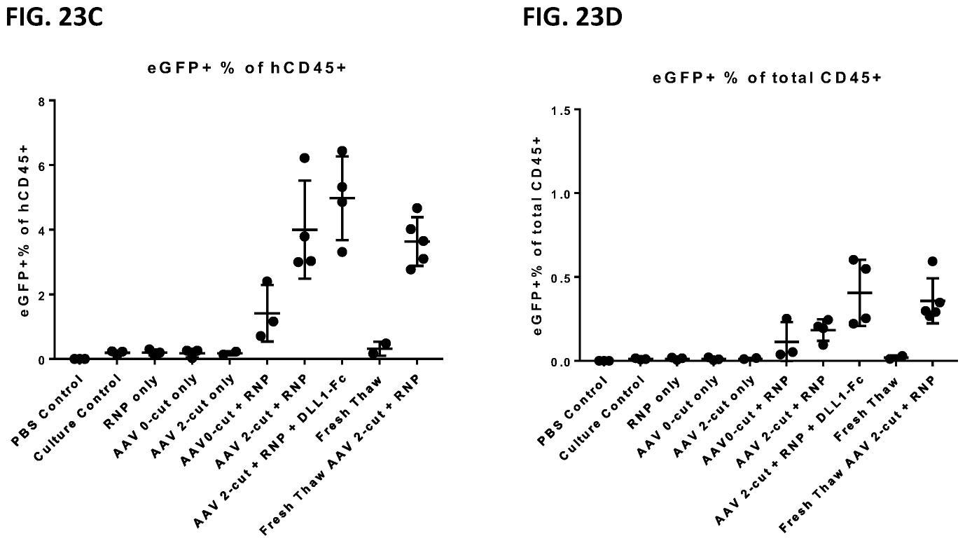

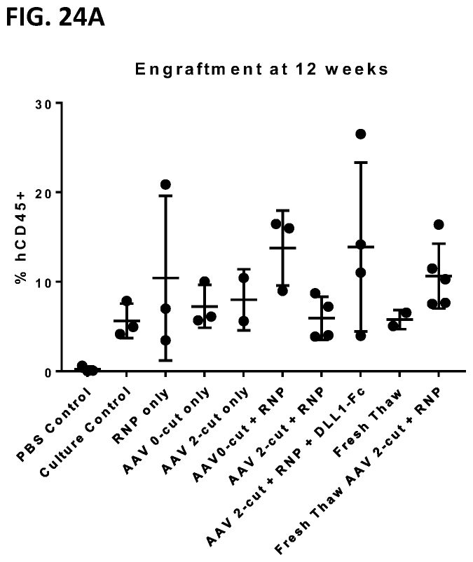

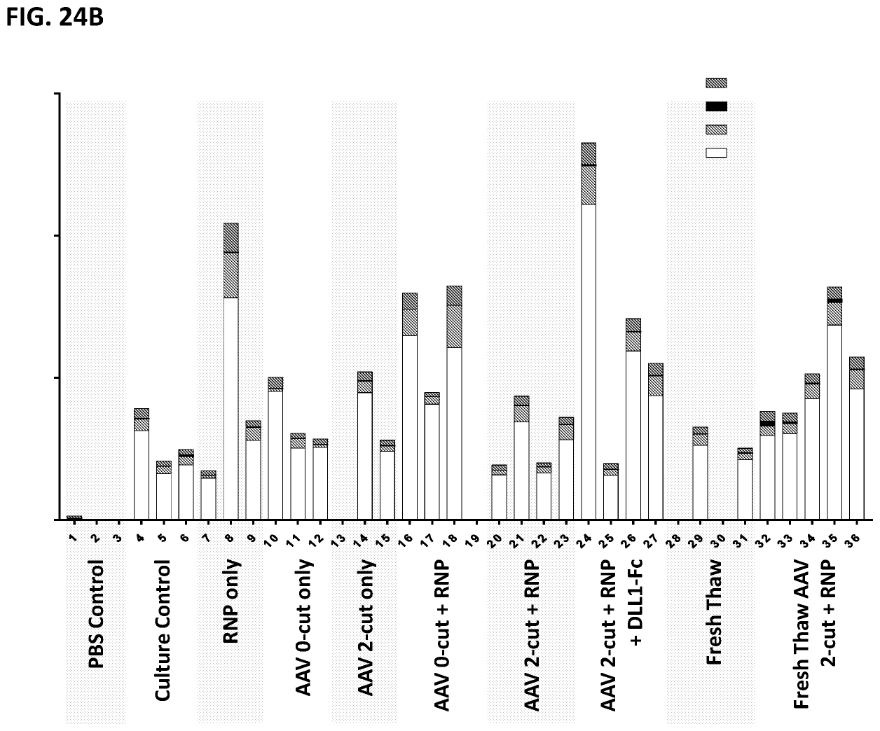

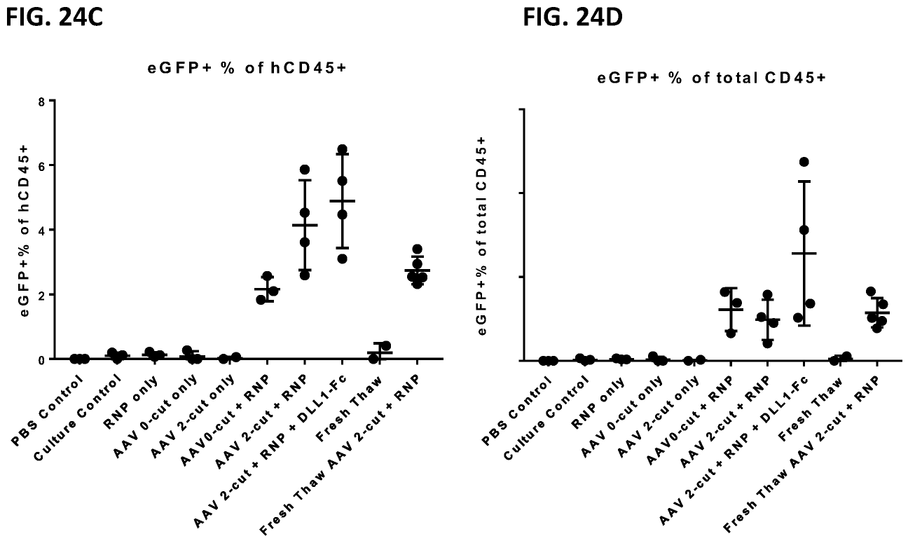

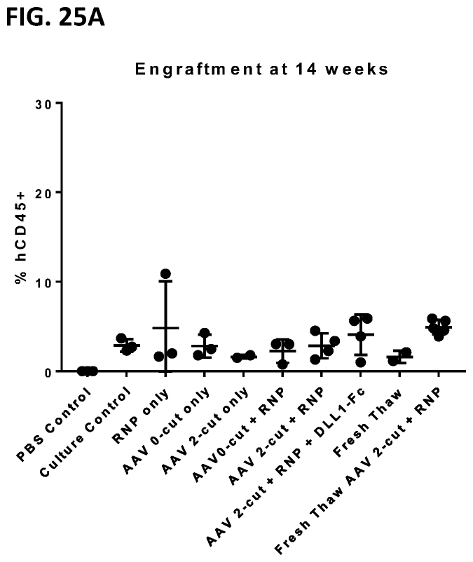

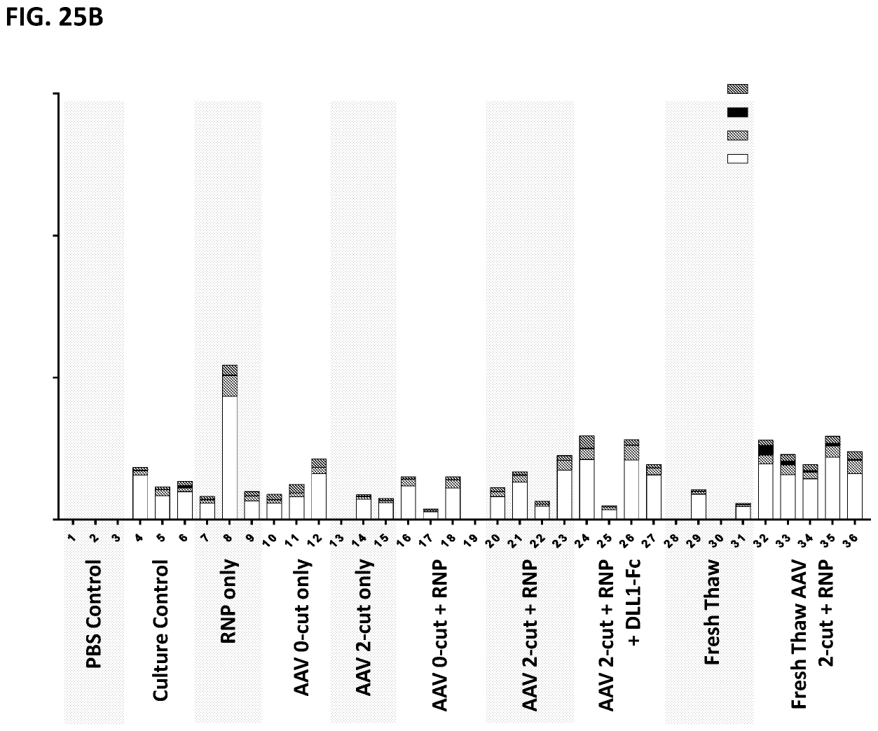

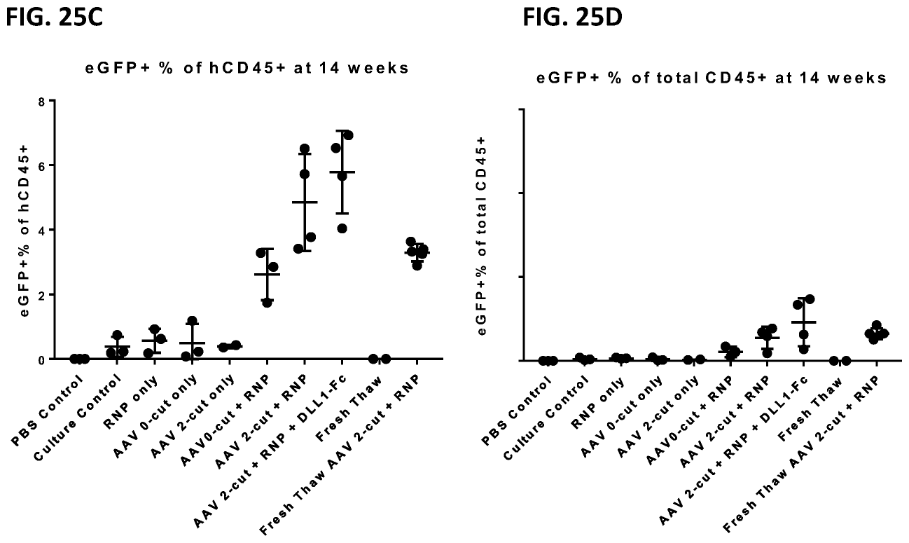

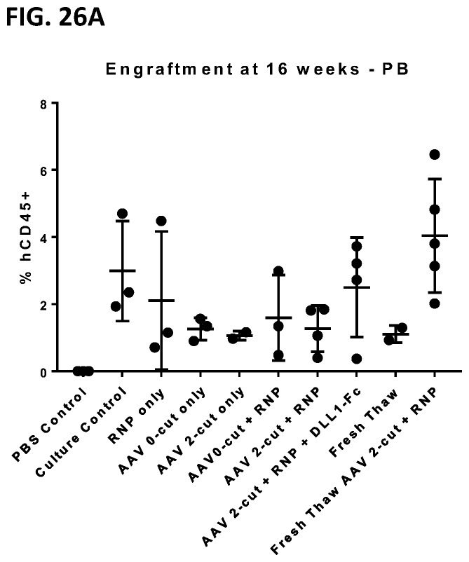

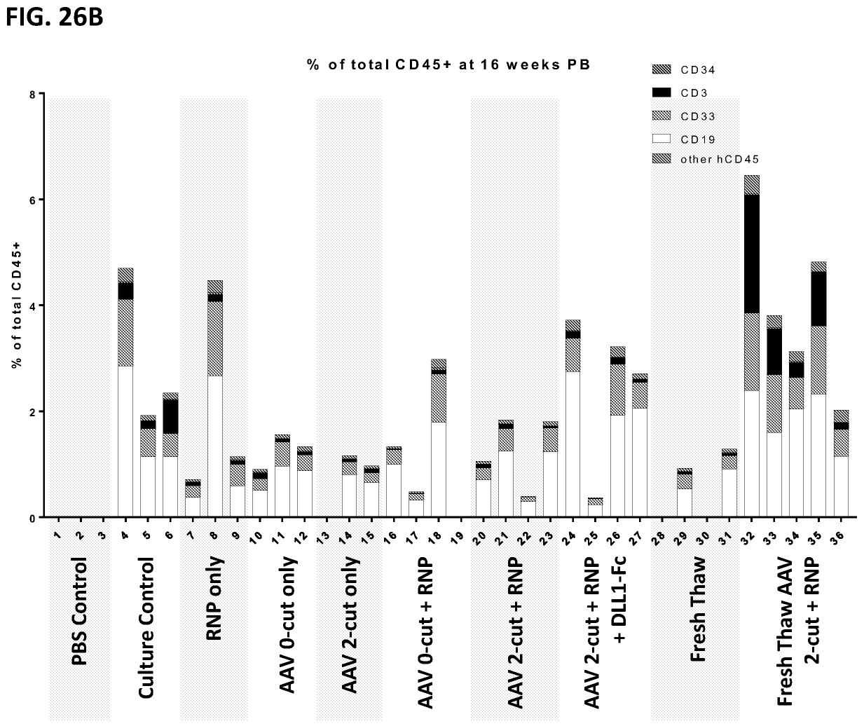

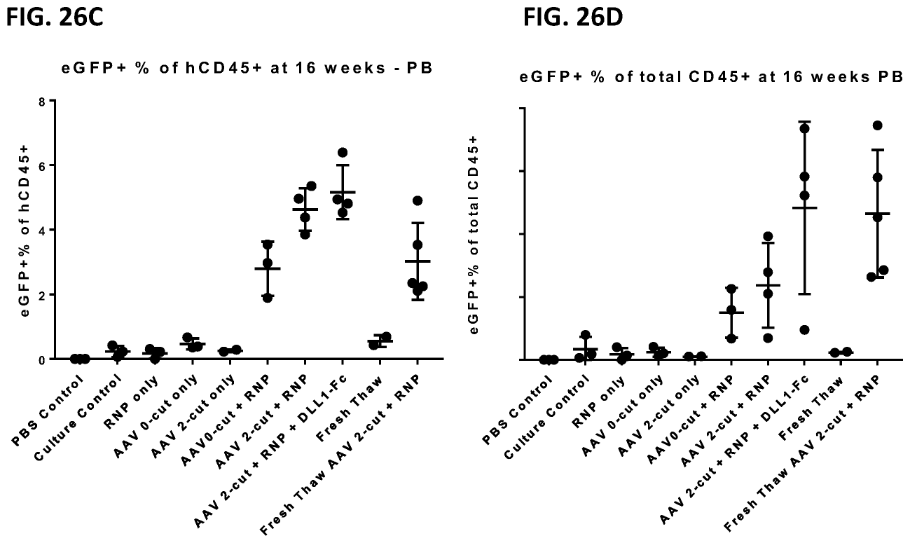

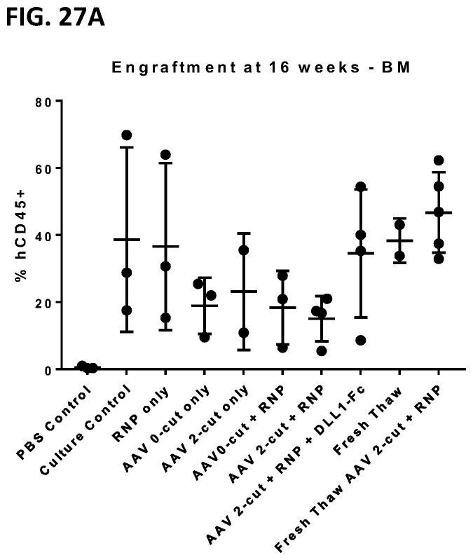

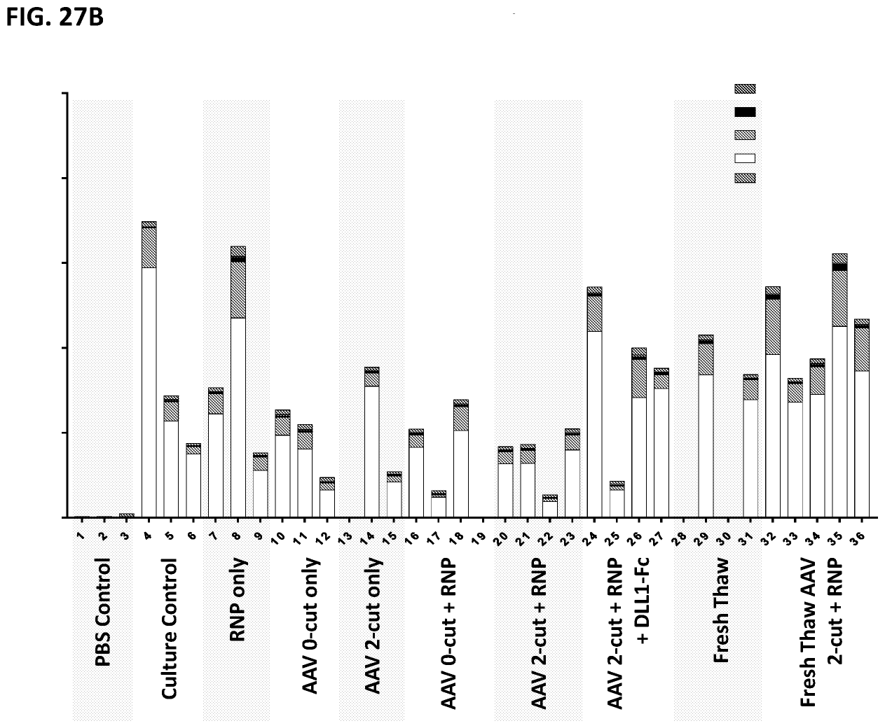

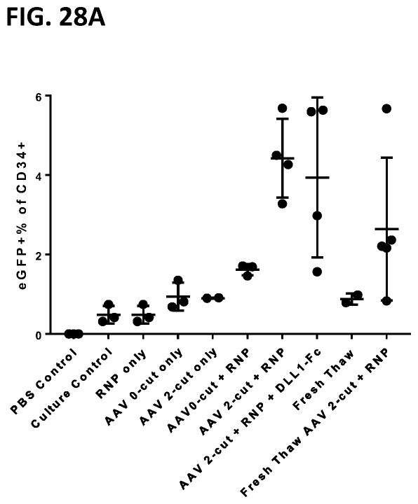

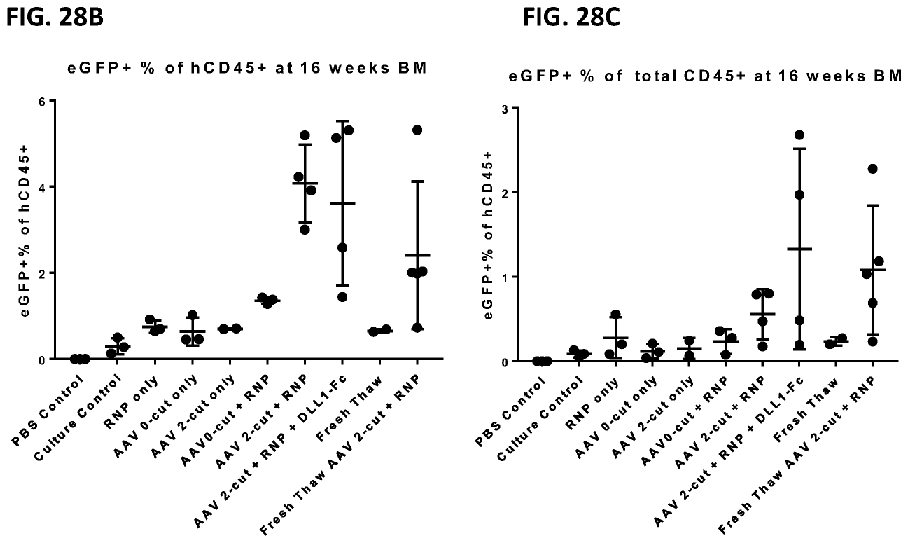

Each mouse received the required number of cells (500,000 cells/mouse for Culture Control and Fresh Thaw, 1,000,000 cells/mouse for other conditions). Results are shown in FIGS. 21, 22A-22D, 23A-23D, 24A-24D, 25A-25D, 26A-26D, 27A, 27B, 28A-28C, and 29A-29C.

The initial editing efficiency of the cells were as follows: Culture Control: 0.01% GFP⁺; RNP only: 0.62% GFP⁺; AAV 0-cut only: 0.42% GFP⁺; AAV2-cut only: 0.73% GFP⁺; AAV 0-cut +RNP: 1.54% GFP⁺; AAV 2-cut +RNP: 3.07% GFP⁺; AAV2-cut +RNP+DLL1-Fc: 2.49% GFP⁺; Fresh Thaw: 0.24% GFP⁺; Fresh Thaw AAV 2-cut +RNP: 2.58% GFP⁺, where the percentages of GFP⁺ signal of Culture Control, RNP only, AAV 0-cut only, AAV 2-cut only, and Fresh Thaw conditions are background noise. These data further demonstrate the improved editing efficiency when employing donor cleavage.

Post 16-week bone marrow engraftment percentages: Culture Control: 38%±27% hCD45⁺; RNP only: 37%±25% hCD45⁺; AAV 0-cut only: 19%±8.4% hCD45⁺; AAV 2-cut only: 23%±17% hCD45⁺; AAV 0-cut +RNP: 18%±11% hCD45⁺; AAV 2-cut +RNP: 15%±6.7% hCD45⁺; AAV 2-cut +RNP+DLL1-Fc: 35%±19% hCD45⁺; Fresh Thaw: 38%±6.6% hCD45⁺; Fresh Thaw AAV 2-cut +RNP: 47%±12% hCD45⁺. The greatest amount of engraftment of hCD45⁺ cells at 16 weeks was observed with the Fresh Thaw condition with AAV 2-cut +RNP.

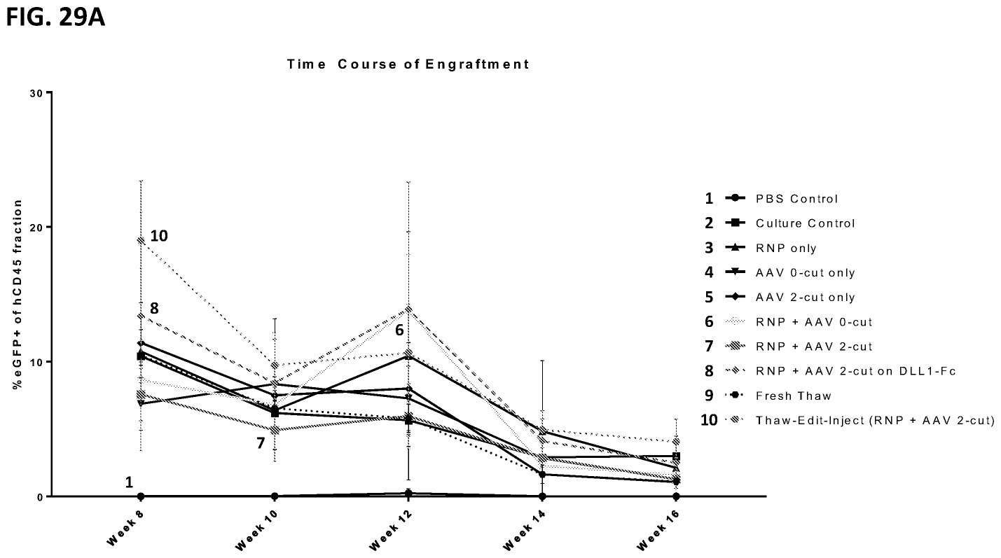

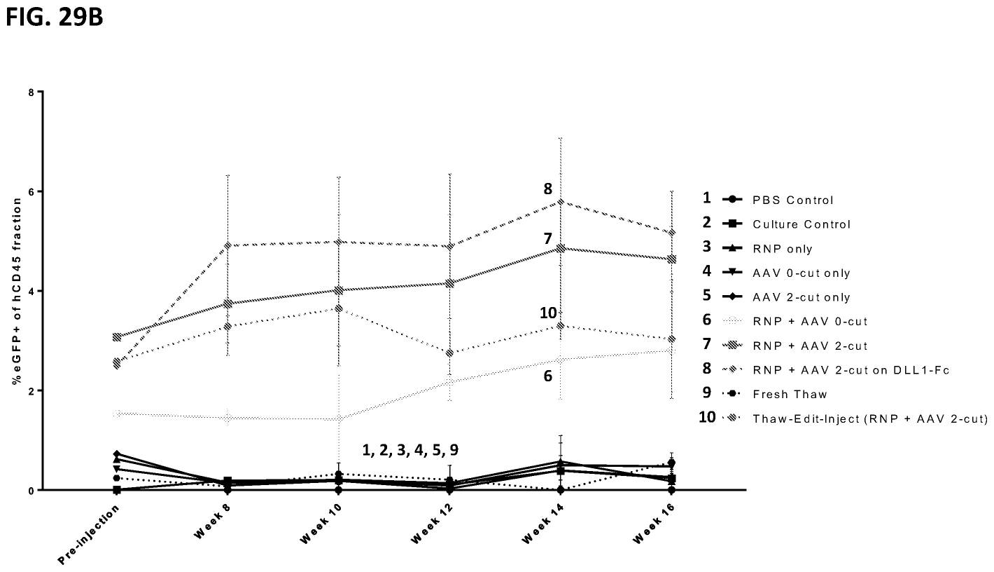

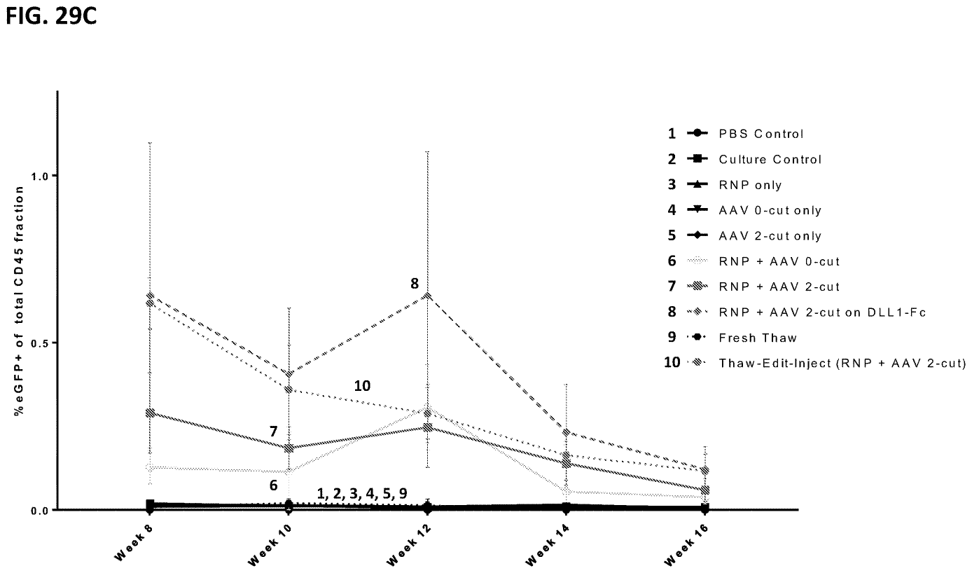

Post 16-week bone marrow GFP⁺ percentages: Culture Control: 0.30%±0.19% GFP⁺; RNP only: 0.76%±0.14% GFP⁺; AAV 0-cut only: 0.64%±0.33% GFP⁺; AAV 2-cut only: 0.70%±0.013% GFP⁺; AAV 0-cut +RNP: 1.36%±0.077% GFP⁺; AAV 2-cut +RNP: 4.1%±0.90% GFP⁺; AAV 2-cut +RNP+DLL1-Fc: 3.6%±1.9% GFP⁺; Fresh Thaw: 0.66%±0.039% GFP⁺; Fresh Thaw AAV 2-cut +RNP: 2.4%±1.7% GFP⁺, where the percentages of GFP⁺ signal of Culture Control, RNP only, AAV 0-cut only, AAV 2-cut only, and Fresh Thaw conditions are background noise. The greatest amount of GFP⁺ cells at 16 weeks was observed with the AAV 2-cut +RNP conditions. As shown in FIG. 29B, the amount of GFP+ cells as a fraction of hCD45+ cells from peripheral bleeds remained steady through week 16 in mice injected with cells treated with RNP+AAV for NHEJ-mediated editing, demonstrating that the edited HSCs were long-term engrafting HSCs.

SEQUENCE LISTING

| SEQ | | |
|---|---|---|
| ID | | |
| NO | Sequence | Description |
| 1 | [CCTGCACTACCAGAGCTAACTCA]¹[CTGACCTCTTCTCTTCCTCCCAC | 0-cut SA-GFP expression |
| | AGG]²GCCTCGAGAGATCTGGCAGCGGA[GGAAGCGGAGCTACTAAC | cassette; |
| | TTCAGCCTGCTGAAGCAGGCTGGAGACGTGGAGGAGAACCCTGGAC | [1-23]¹: 5′ Scrambled |
| | CT]³ACCGGTATGCCTGAACCCTCTAAGTCTGCTCCCGCCCCGAAAAA | Protospacer with PAM; |
| | GGGCTCCAAGAAGGCGGTGACTAAGGCGCAGAAGAAAGGCGGCAA | [24-49]²: Splice Acceptor; |
| | GAAGCGCAAGCGCAGCCGCAAGGAGAGCTATTCCATCTATGTGTAC | [73-138]³: P2A peptide |
| | AAGGTTCTGAAGCAGGTCCACCCTGACACCGGCATTTCGTCCAAGGC | cleavage signal; |
| | CATGGGCATCATGAATTCGTTTGTGAACGACATTTTCGAGCGCATCG | [526-1245]⁴: Venus GFP; |
| | CAGGTGAGGCTTCCCGCCTGGCGCATTACAACAAGCGCTCGACCATC | [1252-1488]⁵: BGH |
| | ACCTCCAGGGAGATCCAGACGGCCGTGCGCCTGCTGCTGCCTGGGG | polyadenylation signal; |
| | AGTTGGCCAAGCACGCCGTGTCCGAGGGTACTAAGGCCGTCACCAA | [1489-1511]⁶: 3′ |
| | GTACACCAGCGCTAAGGAC[ATGGTGAGCAAGGGCGAGGAGCTGTT | Scrambled Protospacer |
| | CACCGGGGTGGTGCCCATCCTGGTCGAGCTGGACGGCGACGTAAAC | with PAM |
| | GGCCACAAGTTCAGCGTGTCCGGCGAGGGCGAGGGCGATGCCACCT | |
| | ACGGCAAGCTGACCCTGAAGTTCATCTGCACCACCGGCAAGCTGCC | |
| | CGTGCCCTGGCCCACCCTCGTGACCACCCTGACCTACGGCGTGCAGT | |
| | GCTTCAGCCGCTACCCCGACCACATGAAGCAGCACGACTTCTTCAAG | |
| | TCCGCCATGCCCGAAGGCTACGTCCAGGAGCGCACCATCTTCTTTAA | |
| | GGATGACGGCAACTACAAGACCCGCGCCGAGGTGAAGTTCGAGGGC | |
| | GACACCCTGGTGAACCGCATCGAGCTGAAGGGCATCGACTTTAAAG | |
| | AGGACGGGAACATCCTGGGGCACAAGCTGGAGTACAACTACAACAG | |
| | CCACAACGTCTATATCATGGCCGACAAGCAGAAGAACGGCATCAAG | |
| | GTGAACTTCAAGATCCGCCACAACATCGAGGACGGCAGCGTGCAGC | |
| | TCGCCGACCACTACCAGCAGAACACCCCCATCGGCGACGGCCCCGT | |
| | GCTGCTGCCCGACAACCACTACCTGAGCACCCAGTCCGCCCTGAGCA | |
| | AAGACCCCAACGAGAAGCGCGATCACATGGTCCTGCTGGAGTTCGT | |
| | GACCGCCGCCGGGATCACTCTCGGCATGGACGAGCTGTACAAGTAA]⁴ | |
| | CATATG[GCTGATCAGCCTCGACTGTGCCTTCTAGTTGCCAGCCATC | |
| | TGTTGTTTGCCCCTCCCCCGTGCCTTCCTTGACCCTGGAAGGTGCCAC | |
| | TCCCACTGTCCTTTCCTAATAAAATGAGGAAATTGCATCGCATTGTC | |
| | TGAGTAGGTGTCATTCTATTCTGGGGGGTGGGGTGGGGCAGGACAG | |
| | CAAGGGGGAGGATTGGGAAGACAATAGCAGGCATGCTGGGGATGC | |
| | GGTGGGCTCTA]⁵[CCTGCACTACCAGAGCTAACTCA]⁶ | |
| 2 | [CCTATCCTGTCCCTAGTGGCCCC]¹[CTGACCTCTTCTCTTCCTCCCAC | 1-cut SA-GFP expression |
| | AGG]²GCCTCGAGAGATCTGGCAGCGGA[GGAAGCGGAGCTACTAAC | cassette; |
| | TTCAGCCTGCTGAAGCAGGCTGGAGACGTGGAGGAGAACCCTGGAC | [1-23]¹: 5′ AAVS1 |
| | CT]³ACCGGTATGCCTGAACCCTCTAAGTCTGCTCCCGCCCCGAAAAA | Protospacer with PAM; |
| | GGGCTCCAAGAAGGCGGTGACTAAGGCGCAGAAGAAAGGCGGCAA | [22-49]²: Splice Acceptor; |
| | GAAGCGCAAGCGCAGCCGCAAGGAGAGCTATTCCATCTATGTGTAC | [73-138]³: P2A peptide |
| | AAGGTTCTGAAGCAGGTCCACCCTGACACCGGCATTTCGTCCAAGGC | cleavage signal; |
| | CATGGGCATCATGAATTCGTTTGTGAACGACATTTTCGAGCGCATCG | [526-1245]⁴: Venus GFP; |
| | CAGGTGAGGCTTCCCGCCTGGCGCATTACAACAAGCGCTCGACCATC | [1252-1488]⁵: BGH |
| | ACCTCCAGGGAGATCCAGACGGCCGTGCGCCTGCTGCTGCCTGGGG | polyadenylation signal; |
| | AGTTGGCCAAGCACGCCGTGTCCGAGGGTACTAAGGCCGTCACCAA | [1489-1511]⁶: 3′ |
| | GTACACCAGCGCTAAGGAC[ATGGTGAGCAAGGGCGAGGAGCTGTT | Scrambled Protospacer |
| | CACCGGGGTGGTGCCCATCCTGGTCGAGCTGGACGGCGACGTAAAC | with PAM |
| | GGCCACAAGTTCAGCGTGTCCGGCGAGGGCGAGGGCGATGCCACCT | |
| | ACGGCAAGCTGACCCTGAAGTTCATCTGCACCACCGGCAAGCTGCC | |
| | CGTGCCCTGGCCCACCCTCGTGACCACCCTGACCTACGGCGTGCAGT | |
| | GCTTCAGCCGCTACCCCGACCACATGAAGCAGCACGACTTCTTCAAG | |
| | TCCGCCATGCCCGAAGGCTACGTCCAGGAGCGCACCATCTTCTTTAA | |
| | GGATGACGGCAACTACAAGACCCGCGCCGAGGTGAAGTTCGAGGGC | |
| | GACACCCTGGTGAACCGCATCGAGCTGAAGGGCATCGACTTTAAAG | |
| | AGGACGGGAACATCCTGGGGCACAAGCTGGAGTACAACTACAACAG | |
| | CCACAACGTCTATATCATGGCCGACAAGCAGAAGAACGGCATCAAG | |
| | GTGAACTTCAAGATCCGCCACAACATCGAGGACGGCAGCGTGCAGC | |
| | TCGCCGACCACTACCAGCAGAACACCCCCATCGGCGACGGCCCCGT | |
| | GCTGCTGCCCGACAACCACTACCTGAGCACCCAGTCCGCCCTGAGCA | |
| | AAGACCCCAACGAGAAGCGCGATCACATGGTCCTGCTGGAGTTCGT | |
| | GACCGCCGCCGGGATCACTCTCGGCATGGACGAGCTGTACAAGTAA]⁴ | |
| | CATATG[GCTGATCAGCCTCGACTGTGCCTTCTAGTTGCCAGCCATC | |
| | TGTTGTTTGCCCCTCCCCCGTGCCTTCCTTGACCCTGGAAGGTGCCAC | |
| | TCCCACTGTCCTTTCCTAATAAAATGAGGAAATTGCATCGCATTGTC | |
| | TGAGTAGGTGTCATTCTATTCTGGGGGGTGGGGTGGGGCAGGACAG | |
| | CAAGGGGGAGGATTGGGAAGACAATAGCAGGCATGCTGGGGATGC | |
| | GGTGGGCTCTA]⁵[CCTGCACTACCAGAGCTAACTCA]⁶ | |
| 3 | [CCTATCCTGTCCCTAGTGGCCCC]¹[CTGACCTCTTCTCTTCCTCCCAC | 2-cut SA-GFP expression |
| | AGG]²GCCTCGAGAGATCTGGCAGCGGA[GGAAGCGGAGCTACTAAC | cassette; |
| | TTCAGCCTGCTGAAGCAGGCTGGAGACGTGGAGGAGAACCCTGGAC | [1-23]¹: 5′ AAVS1 |
| | CT]³ACCGGTATGCCTGAACCCTCTAAGTCTGCTCCCGCCCCGAAAAA | Protospacer with PAM; |
| | GGGCTCCAAGAAGGCGGTGACTAAGGCGCAGAAGAAAGGCGGCAA | [24-49]²: Splice Acceptor; |
| | GAAGCGCAAGCGCAGCCGCAAGGAGAGCTATTCCATCTATGTGTAC | [73-138]³: P2A peptide |
| | AAGGTTCTGAAGCAGGTCCACCCTGACACCGGCATTTCGTCCAAGGC | cleavage signal; |
| | CATGGGCATCATGAATTCGTTTGTGAACGACATTTTCGAGCGCATCG | [526-1245]⁴: Venus GFP; |
| | CAGGTGAGGCTTCCCGCCTGGCGCATTACAACAAGCGCTCGACCATC | [1252-1488]⁵: BGH |
| | ACCTCCAGGGAGATCCAGACGGCCGTGCGCCTGCTGCTGCCTGGGG | polyadenylation signal; |
| | AGTTGGCCAAGCACGCCGTGTCCGAGGGTACTAAGGCCGTCACCAA | [1489-1511]⁶: 3′ AAVS1 |
| | GTACACCAGCGCTAAGGAC[ATGGTGAGCAAGGGCGAGGAGCTGTT | Protospacer with PAM |
| | CACCGGGGTGGTGCCCATCCTGGTCGAGCTGGACGGCGACGTAAAC | |
| | GGCCACAAGTTCAGCGTGTCCGGCGAGGGCGAGGGCGATGCCACCT | |
| | ACGGCAAGCTGACCCTGAAGTTCATCTGCACCACCGGCAAGCTGCC | |
| | CGTGCCCTGGCCCACCCTCGTGACCACCCTGACCTACGGCGTGCAGT | |
| | GCTTCAGCCGCTACCCCGACCACATGAAGCAGCACGACTTCTTCAAG | |
| | TCCGCCATGCCCGAAGGCTACGTCCAGGAGCGCACCATCTTCTTTAA | |
| | GGATGACGGCAACTACAAGACCCGCGCCGAGGTGAAGTTCGAGGGC | |
| | GACACCCTGGTGAACCGCATCGAGCTGAAGGGCATCGACTTTAAAG | |
| | AGGACGGGAACATCCTGGGGCACAAGCTGGAGTACAACTACAACAG | |
| | CCACAACGTCTATATCATGGCCGACAAGCAGAAGAACGGCATCAAG | |
| | GTGAACTTCAAGATCCGCCACAACATCGAGGACGGCAGCGTGCAGC | |
| | TCGCCGACCACTACCAGCAGAACACCCCCATCGGCGACGGCCCCGT | |
| | GCTGCTGCCCGACAACCACTACCTGAGCACCCAGTCCGCCCTGAGCA | |
| | AAGACCCCAACGAGAAGCGCGATCACATGGTCCTGCTGGAGTTCGT | |
| | GACCGCCGCCGGGATCACTCTCGGCATGGACGAGCTGTACAAGTAA]⁴ | |
| | CATATG[GCTGATCAGCCTCGACTGTGCCTTCTAGTTGCCAGCCATC | |
| | TGTTGTTTGCCCCTCCCCCGTGCCTTCCTTGACCCTGGAAGGTGCCAC | |
| | TCCCACTGTCCTTTCCTAATAAAATGAGGAAATTGCATCGCATTGTC | |
| | TGAGTAGGTGTCATTCTATTCTGGGGGGTGGGGTGGGGCAGGACAG | |
| | CAAGGGGGAGGATTGGGAAGACAATAGCAGGCATGCTGGGGATGC | |
| | GGTGGGCTCTA]⁵[CCTATCCTGTCCCTAGTGGCCCC]⁶ | |
| 4 | [CCTGCAGGCAGCTGCGCGCTCGCTCGCTCACTGAGGCCGCCCGGGC | 0-cut SA-GFP scAAV; |
| | AAAGCCCGGGCGTCGGGCGACCTTTGGTCGCCCGGCCTCAGTGAGC | [1-117]¹: Mutant ITR; |
| | GAGCGAGCGCGCAGAGAGGGAGTGG]¹ACGCGTAGGCCTAAGCTTGG | [188-1695]²: 0-cut SA- |
| | TACCGGATCCACTAGTAACGGCCGCCAGTGTGCTGGAATTCGCCCTT | GFP expression cassette; |
| | CTT[TGAGTTAGCTCTGGTAGTGCAGGTAGAGCCCACCGCATCCCCA | [1752-1892]³: Wild-type |
| | GCATGCCTGCTATTGTCTTCCCAATCCTCCCCCTTGCTGTCCTGCCCC | ITR |
| | ACCCCACCCCCCAGAATAGAATGACACCTACTCAGACAATGCGATG | |
| | CAATTTCCTCATTTTATTAGGAAAGGACAGTGGGAGTGGCACCTTCC | |
| | AGGGTCAAGGAAGGCACGGGGGAGGGGCAAACAACAGATGGCTGG | |
| | CAACTAGAAGGCACAGTCGAGGCTGATCAGCCATATGTTACTTGTAC | |
| | AGCTCGTCCATGCCGAGAGTGATCCCGGCGGCGGTCACGAACTCCA | |
| | GCAGGACCATGTGATCGCGCTTCTCGTTGGGGTCTTTGCTCAGGGCG | |
| | GACTGGGTGCTCAGGTAGTGGTTGTCGGGCAGCAGCACGGGGCCGT | |
| | CGCCGATGGGGGTGTTCTGCTGGTAGTGGTCGGCGAGCTGCACGCTG | |
| | CCGTCCTCGATGTTGTGGCGGATCTTGAAGTTCACCTTGATGCCGTT | |
| | CTTCTGCTTGTCGGCCATGATATAGACGTTGTGGCTGTTGTAGTTGTA | |
| | CTCCAGCTTGTGCCCCAGGATGTTCCCGTCCTCTTTAAAGTCGATGC | |
| | CCTTCAGCTCGATGCGGTTCACCAGGGTGTCGCCCTCGAACTTCACC | |
| | TCGGCGCGGGTCTTGTAGTTGCCGTCATCCTTAAAGAAGATGGTGCG | |
| | CTCCTGGACGTAGCCTTCGGGCATGGCGGACTTGAAGAAGTCGTGCT | |
| | GCTTCATGTGGTCGGGGTAGCGGCTGAAGCACTGCACGCCGTAGGT | |
| | CAGGGTGGTCACGAGGGTGGGCCAGGGCACGGGCAGCTTGCCGGTG | |
| | GTGCAGATGAACTTCAGGGTCAGCTTGCCGTAGGTGGCATCGCCCTC | |
| | GCCCTCGCCGGACACGCTGAACTTGTGGCCGTTTACGTCGCCGTCCA | |
| | GCTCGACCAGGATGGGCACCACCCCGGTGAACAGCTCCTCGCCCTTG | |
| | CTCACCATGTCCTTAGCGCTGGTGTACTTGGTGACGGCCTTAGTACC | |
| | CTCGGACACGGCGTGCTTGGCCAACTCCCCAGGCAGCAGCAGGCGC | |
| | ACGGCCGTCTGGATCTCCCTGGAGGTGATGGTCGAGCGCTTGTTGTA | |
| | ATGCGCCAGGCGGGAAGCCTCACCTGCGATGCGCTCGAAAATGTCG | |
| | TTCACAAACGAATTCATGATGCCCATGGCCTTGGACGAAATGCCGGT | |
| | GTCAGGGTGGACCTGCTTCAGAACCTTGTACACATAGATGGAATAG | |
| | CTCTCCTTGCGGCTGCGCTTGCGCTTCTTGCCGCCTTTCTTCTGCGCC | |
| | TTAGTCACCGCCTTCTTGGAGCCCTTTTTCGGGGCGGGAGCAGACTT | |
| | AGAGGGTTCAGGCATACCGGTAGGTCCAGGGTTCTCCTCCACGTCTC | |
| | CAGCCTGCTTCAGCAGGCTGAAGTTAGTAGCTCCGCTTCCTCCGCTG | |
| | CCAGATCTCTCGAGGCCCTGTGGGAGGAAGAGAAGAGGTCAGTGAG | |
| | TTAGCTCTGGTAGTGC]²AGGTACCAAGGGCGAATTCTGCAGATATCC | |
| | ATCACACTGGCTTAATTAAGGGCCGC[AGGAACCCCTAGTGATGGAG | |
| | TTGGCCACTCCCTCTCTGCGCGCTCGCTCGCTCACTGAGGCCGGGCG | |
| | ACCAAAGGTCGCCCGACGCCCGGGCTTTGCCCGGGCGGCCTCAGTG | |
| | AGCGAGCGAGCGCGCAGCTGCCTGCAGG]³ | |
| 5 | [CCTGCAGGCAGCTGCGCGCTCGCTCGCTCACTGAGGCCGGGCGACC | 2-cut SA-GFP scAAV; |
| | AAAGGTCGCCCGACGCCCGGGCTTTGCCCGCCCGGCCTCAGTGAGC | [1-117]¹: Mutant ITR; |
| | GAGCGAGCGCGCAGAGAGGGAGTGG]¹ACGCGTAGGCCTAAGCTTGG | [212-1699]²: 2-cut SA- |
| | TACCGGATCCACTAGTAACGGCCGCCAGTGTGCTGGAATTCGCCCTT | GFP expression cassette; |
| | GGTACCTATCCTGTCCCTAGTGGCCCC[CTGACCTCTTCTCTTCCTCCC | [1755-1895]³: Wild-type |
| | ACAGGGCCTCGAGAGATCTGGCAGCGGAGGAAGCGGAGCTACTAAC | ITR |
| | TTCAGCCTGCTGAAGCAGGCTGGAGACGTGGAGGAGAACCCTGGAC | |
| | CTACCGGTATGCCTGAACCCTCTAAGTCTGCTCCCGCCCCGAAAAAG | |
| | GGCTCCAAGAAGGCGGTGACTAAGGCGCAGAAGAAAGGCGGCAAG | |
| | AAGCGCAAGCGCAGCCGCAAGGAGAGCTATTCCATCTATGTGTACA | |
| | AGGTTCTGAAGCAGGTCCACCCTGACACCGGCATTTCGTCCAAGGCC | |
| | ATGGGCATCATGAATTCGTTTGTGAACGACATTTTCGAGCGCATCGC | |
| | AGGTGAGGCTTCCCGCCTGGCGCATTACAACAAGCGCTCGACCATC | |
| | ACCTCCAGGGAGATCCAGACGGCCGTGCGCCTGCTGCTGCCTGGGG | |
| | AGTTGGCCAAGCACGCCGTGTCCGAGGGTACTAAGGCCGTCACCAA | |
| | GTACACCAGCGCTAAGGACATGGTGAGCAAGGGCGAGGAGCTGTTC | |
| | ACCGGGGTGGTGCCCATCCTGGTCGAGCTGGACGGCGACGTAAACG | |
| | GCCACAAGTTCAGCGTGTCCGGCGAGGGCGAGGGCGATGCCACCTA | |
| | CGGCAAGCTGACCCTGAAGTTCATCTGCACCACCGGCAAGCTGCCC | |
| | GTGCCCTGGCCCACCCTCGTGACCACCCTGACCTACGGCGTGCAGTG | |
| | CTTCAGCCGCTACCCCGACCACATGAAGCAGCACGACTTCTTCAAGT | |
| | CCGCCATGCCCGAAGGCTACGTCCAGGAGCGCACCATCTTCTTTAAG | |
| | GATGACGGCAACTACAAGACCCGCGCCGAGGTGAAGTTCGAGGGCG | |
| | ACACCCTGGTGAACCGCATCGAGCTGAAGGGCATCGACTTTAAAGA | |
| | GGACGGGAACATCCTGGGGCACAAGCTGGAGTACAACTACAACAGC | |
| | CACAACGTCTATATCATGGCCGACAAGCAGAAGAACGGCATCAAGG | |
| | TGAACTTCAAGATCCGCCACAACATCGAGGACGGCAGCGTGCAGCT | |
| | CGCCGACCACTACCAGCAGAACACCCCCATCGGCGACGGCCCCGTG | |
| | CTGCTGCCCGACAACCACTACCTGAGCACCCAGTCCGCCCTGAGCAA | |
| | AGACCCCAACGAGAAGCGCGATCACATGGTCCTGCTGGAGTTCGTG | |
| | ACCGCCGCCGGGATCACTCTCGGCATGGACGAGCTGTACAAGTAAC | |
| | ATATGGCTGATCAGCCTCGACTGTGCCTTCTAGTTGCCAGCCATCTG | |
| | TTGTTTGCCCCTCCCCCGTGCCTTCCTTGACCCTGGAAGGTGCCACTC | |
| | CCACTGTCCTTTCCTAATAAAATGAGGAAATTGCATCGCATTGTCTG | |
| | AGTAGGTGTCATTCTATTCTGGGGGGTGGGGTGGGGCAGGACAGCA | |
| | AGGGGGAGGATTGGGAAGACAATAGCAGGCATGCTGGGGATGCGG | |
| | TGGGCTCTACCTATCCTGTCCCTAGTGGCCCC]²AAGCTTAAGGGCGA | |
| | ATTCTGCAGATATCCATCACACTGGCTTAATTAAGGGCCGC[AGGAA | |
| | CCCCTAGTGATGGAGTTGGCCACTCCCTCTCTGCGCGCTCGCTCGCT | |
| | CACTGAGGCCGGGCGACCAAAGGTCGCCCGACGCCCGGGCTTTGCC | |
| | CGGGCGGCCTCAGTGAGCGAGCGAGCGCGCAGCTGCCTGCAGG]³ | |
| 6 | [CCTGCAGGCAGCTGCGCGCTCGCTCGCTCACTGAGGCCGCCCGGGC | 2-cut SA-tNGFR scAAV; |
| | AAAGCCCGGGCGTCGGGCGACCTTTGGTCGCCCGGCCTCAGTGAGC | [1-117]¹: Mutant ITR; |
| | GAGCGAGCGCGCAGAGAGGGAGTGG]¹ACGCGTAGGCCTAAGCTTGG | [212-1417]²: 2-cut SA- |
| | TACCGGATCCACTAGTAACGGCCGCCAGTGTGCTGGAATTCGCCCTT | tNGFR transgene; |
| | GGTACCTATCCTGTCCCTAGTGGCCCC[CTGACCTCTTCTCTTCCTCCC | [1473-1613]³: Wild-type |
| | ACAGGGCCTCGAGAGATCTGGCAGCGGAGGAAGCGGAGCTACTAAC | ITR |
| | TTCAGCCTGCTGAAGCAGGCTGGAGACGTGGAGGAGAACCCTGGAC | |
| | CTACCGGTATGGGGGCAGGTGCCACCGGCCGCGCCATGGACGGGCC | |
| | GCGCCTGCTGCTGTTGCTGCTTCTGGGGGTGTCCCTTGGAGGTGCCA | |
| | AGGAGGCATGCCCCACAGGCCTGTACACACACAGCGGTGAGTGCTG | |
| | CAAAGCCTGCAACCTGGGCGAGGGTGTGGCCCAGCCTTGTGGAGCC | |
| | AACCAGACCGTGTGTGAGCCCTGCCTGGACAGCGTGACGTTCTCCGA | |
| | CGTGGTGAGCGCGACCGAGCCGTGCAAGCCGTGCACCGAGTGCGTG | |
| | GGGCTCCAGAGCATGTCGGCGCCGTGCGTGGAGGCCGACGACGCCG | |
| | TGTGCCGCTGCGCCTACGGCTACTACCAGGATGAGACGACTGGGCG | |
| | CTGCGAGGCGTGCCGCGTGTGCGAGGCGGGCTCGGGCCTCGTGTTCT | |
| | CCTGCCAGGACAAGCAGAACACCGTGTGCGAGGAGTGCCCCGACGG | |
| | CACGTATTCCGACGAGGCCAACCACGTGGACCCGTGCCTGCCCTGCA | |
| | CCGTGTGCGAGGACACCGAGCGCCAGCTCCGCGAGTGCACACGCTG | |
| | GGCCGACGCCGAGTGCGAGGAGATCCCTGGCCGTTGGATTACACGG | |
| | TCCACACCCCCAGAGGGCTCGGACAGCACAGCCCCCAGCACCCAGG | |
| | AGCCTGAGGCACCTCCAGAACAAGACCTCATAGCCAGCACGGTGGC | |
| | AGGTGTGGTGACCACAGTGATGGGCAGCTCCCAGCCCGTGGTGACC | |
| | CGAGGCACCACCGACAACCTCATCCCTGTCTATTGCTCCATCCTGGC | |
| | TGCTGTGGTTGTGGGCCTTGTGGCCTACATAGCCTTCTGACATATGG | |
| | CTGATCAGCCTCGACTGTGCCTTCTAGTTGCCAGCCATCTGTTGTTTG | |
| | CCCCTCCCCCGTGCCTTCCTTGACCCTGGAAGGTGCCACTCCCACTG | |
| | TCCTTTCCTAATAAAATGAGGAAATTGCATCGCATTGTCTGAGTAGG | |
| | TGTCATTCTATTCTGGGGGGTGGGGTGGGGCAGGACAGCAAGGGGG | |
| | AGGATTGGGAAGACAATAGCAGGCATGCTGGGGATGCGGTGGGCTC | |
| | TACCTATCCTGTCCCTAGTGGCCCC]²AAGCTTAAGGGCGAATTCTGC | |
| | AGATATCCATCACACTGGCTTAATTAAGGGCCGC[AGGAACCCCTAG | |
| | TGATGGAGTTGGCCACTCCCTCTCTGCGCGCTCGCTCGCTCACTGAG | |
| | GCCGGGCGACCAAAGGTCGCCCGACGCCCGGGCTTTGCCCGGGCGG | |
| | CCTCAGTGAGCGAGCGAGCGCGCAGCTGCCTGCAGG]³ | |
| 7 | [CCTGCAGGCAGCTGCGCGCTCGCTCGCTCACTGAGGCCGCCCGGGC | HDR-TI SA-GFP ssAAV; |
| | AAAGCCCGGGCGTCGGGCGACCTTTGGTCGCCCGGCCTCAGTGAGC | [1-141]¹: 5′ wild-type ITR; |
| | GAGCGAGCGCGCAGAGAGGGAGTGGCCAACTCCATCACTAGGGGTT | [269-1067]²: 5 Homology |
| | CCT]¹GCGGCCAATTCAGTCGATAACTATAACGGTCCTAAGGTAGCGA | Arm; |
| | TTTAAATACGCGCTCTCTTAAGGTAGCCCCGGGACGCGTCAATTGAG | [1085-2541]³: SA-GFP |
| | ATCTGGATCCGGTACCTGCACTACCAGAGCTAACTCA[TGCTTTCTCT | expression cassette; |
| | GACCAGCATTCTCTCCCCTGGGCCTGTGCCGCTTTCTGTCTGCAGCTT | [2543-3356]⁴: 3′ |
| | GTGGCCTGGGTCACCTCTACGGCTGGCCCAGATCCTTCCCTGCCGCC | Homology Arm; |
| | TCCTTCAGGTTCCGTCTTCCTCCACTCCCTCTTCCCCTTGCTCTCTGCT | [3451-3591]⁵: 3′ wild-type |
| | GTGTTGCTGCCCAAGGATGCTCTTTCCGGAGCACTTCCTTCTCGGCG | ITR |
| | CTGCACCACGTGATGTCCTCTGAGCGGATCCTCCCCGTGTCTGGGTC | |
| | CTCTCCGGGCATCTCTCCTCCCTCACCCAACCCCATGCCGTCTTCACT | |
| | CGCTGGGTTCCCTTTTCCTTCTCCTTCTGGGGCCTGTGCCATCTCTCG | |
| | TTTCTTAGGATGGCCTTCTCCGACGGATGTCTCCCTTGCGTCCCGCCT | |
| | CCCCTTCTTGTAGGCCTGCATCATCACCGTTTTTCTGGACAACCCCAA | |
| | AGTACCCCGTCTCCCTGGCTTTAGCCACCTCTCCATCCTCTTGCTTTC | |
| | TTTGCCTGGACACCCCGTTCTCCTGTGGATTCGGGTCACCTCTCACTC | |
| | CTTTCATTTGGGCAGCTCCCCTACCCCCCTTACCTCTCTAGTCTGTGC | |
| | TAGCTCTTCCAGCCCCCTGTCATGGCATCTTCCAGGGGTCCGAGAGC | |
| | TCAGCTAGTCTTCTTCCTCCAACCCGGGCCCCTATGTCCACTTCAGG | |
| | ACAGCATGTTTGCTGCCTCCAGGGATCCTGTGTCCCCGAGCTGGGAC | |
| | CACCTTATATTCCCAGGGCCGGTTAATGTGGCTCTGGTTCTGGGTAC | |
| | TTTTATCTGTCCCCTCCACCCCACAGT]²GGGGCCACTAGGGACAG[CT | |
| | GACCTCTTCTCTTCCTCCCACAGGGCTCTGGCAGCGGAGGAAGCGGA | |
| | GCTACTAACTTTAGTCTGCTTAATCAAGCTGGAGACGTGGAGGAGA | |
| | ACCCTGGACCTACCGGTATGCCTGAACCCTCTAAGTCTGCTCCCGCC | |
| | CCGAAAAAGGGCTCCAAGAAGGCGGTGACTAAGGCGCAGAAGAAA | |
| | GGCGGCAAGAAGCGCAAGCGCAGCCGCAAGGAGAGCTATTCCATCT | |
| | ATGTGTACAAGGTTCTGAAGCAGGTCCACCCTGACACCGGCATTTCG | |
| | TCCAAGGCCATGGGCATCATGAATTCGTTTGTGAACGACATTTTCGA | |
| | GCGCATCGCAGGTGAGGCTTCCCGCCTGGCGCATTACAACAAGCGC | |
| | TCGACCATCACCTCCAGGGAGATCCAGACGGCCGTGCGCCTGCTGCT | |
| | GCCTGGGGAGTTGGCCAAGCACGCCGTGTCCGAGGGTACTAAGGCC | |
| | GTCACCAAGTACACCAGCGCTAAGGACATGGTGAGCAAGGGCGAGG | |
| | AGCTGTTCACCGGGGTGGTGCCCATCCTGGTCGAGCTGGACGGCGA | |
| | CGTAAACGGCCACAAGTTCAGCGTGTCCGGCGAGGGCGAGGGCGAT | |
| | GCCACCTACGGCAAGCTGACCCTGAAGTTCATCTGCACCACCGGCA | |
| | AGCTGCCCGTGCCCTGGCCCACCCTCGTGACCACCCTGACCTACGGC | |
| | GTGCAGTGCTTCAGCCGCTACCCCGACCACATGAAGCAGCACGACTT | |
| | CTTCAAGTCCGCCATGCCCGAAGGCTACGTCCAGGAGCGCACCATCT | |
| | TCTTTAAGGATGACGGCAACTACAAGACCCGCGCCGAGGTGAAGTT | |
| | CGAGGGCGACACCCTGGTGAACCGCATCGAGCTGAAGGGCATCGAC | |
| | TTTAAAGAGGACGGGAACATCCTGGGGCACAAGCTGGAGTACAACT | |
| | ACAACAGCCACAACGTCTATATCATGGCCGACAAGCAGAAGAACGG | |
| | CATCAAGGTGAACTTCAAGATCCGCCACAACATCGAGGACGGCAGC | |
| | GTGCAGCTCGCCGACCACTACCAGCAGAACACCCCCATCGGCGACG | |
| | GCCCCGTGCTGCTGCCCGACAACCACTACCTGAGCACCCAGTCCGCC | |
| | CTGAGCAAAGACCCCAACGAGAAGCGCGATCACATGGTCCTGCTGG | |
| | AGTTCGTGACCGCCGCCGGGATCACTCTCGGCATGGACGAGCTGTAC | |
| | AAGTAACATATGGCTGATCAGCCTCGACTGTGCCTTCTAGTTGCCAG | |
| | CCATCTGTTGTTTGCCCCTCCCCCGTGCCTTCCTTGACCCTGGAAGGT | |
| | GCCACTCCCACTGTCCTTTCCTAATAAAATGAGGAAATTGCATCGCA | |
| | TTGTCTGAGTAGGTGTCATTCTATTCTGGGGGGTGGGGTGGGGCAGG | |
| | ACAGCAAGGGGGAGGATTGGGAAGACAATAGCAGGCATGCTGGGG | |
| | ATGCGGTGGGCTCTAT]³G[ATTGGTGACAGAAAAGCCCCATCCTTAG | |
| | GCCTCCTCCTTCCTAGTCTCCTGATATTGGGTCTAACCCCCACCTCCT | |
| | GTTAGGCAGATTCCTTATCTGGTGACACACCCCCATTTCCTGGAGCC | |
| | ATCTCTCTCCTTGCCAGAACCTCTAAGGTTTGCTTACGATGGAGCCA | |
| | GAGAGGATCCTGGGAGGGAGAGCTTGGCAGGGGGTGGGAGGGAAG | |
| | GGGGGGATGCGTGACCTGCCCGGTTCTCAGTGGCCACCCTGCGCTAC | |
| | CCTCTCCCAGAACCTGAGCTGCTCTGACGCGGCTGTCTGGTGCGTTT | |
| | CACTGATCCTGGTGCTGCAGCTTCCTTACACTTCCCAAGAGGAGAAG | |
| | CAGTTTGGAAAAACAAAATCAGAATAAGTTGGTCCTGAGTTCTAACT | |
| | TTGGCTCTTCACCTTTCTAGTCCCCAATTTATATTGTTCCTCCGTGCG | |
| | TCAGTTTTACCTGTGAGATAAGGCCAGTAGCCAGCCCCGTCCTGGCA | |
| | GGGCTGTGGTGAGGAGGGGGGTGTCCGTGTGGAAAACTCCCTTTGT | |
| | GAGAATGGTGCGTCCTAGGTGTTCACCAGGTCGTGGCCGCCTCTACT | |
| | CCCTTTCTCTTTCTCCATCCTTCTTTCCTTAAAGAGTCCCCAGTGCTA | |
| | TCTGGGACATATTCCTCCGCCCAGAGCAGGGTCCCGCTTCCCTAAGG | |
| | CCCTGCTCTGGGCTTCTGGGTTTGAGTCCTTGGCAAGCCCAGGAGAG | |
| | GCGCTCAGGCTTCCCTGTCCCCCTTCCTCGTCCACCATCTCATGCCCC | |
| | TGGCTCTCCTGCCCCTTCCCTACAGGGGTTCCT]⁴CACTACCAGAGC | |
| | TAACTCAAAGCTTCTAGATATCCTCTCTTAAGGTAGCATCGAGATTT | |
| | AAATTAGGGATAACAGGGTAATGGCGCGGGCCGC[AGGAACCCCTA | |
| | GTGATGGAGTTGGCCACTCCCTCTCTGCGCGCTCGCTCGCTCACTGA | |
| | GGCCGGGCGACCAAAGGTCGCCCGACGCCCGGGCTTTGCCCGGGCG | |
| | GCCTCAGTGAGCGAGCGAGCGCGCAGCTGCCTGCAGG]⁵ | |
| 8 | GGGGCCACUAGGGACAGGAUGUUUUAGAGCUAGAAAUAGCAAGU | AAVS1 sgRNA |
| | UAAAAUAAGGCUAGUCCGUUAUCAACUUGAAAAAGUGGCACCGAG | |
| | UCGGUGCUUUU | |
| 9 | GGGGCCACTAGGGACAGGATTGG | Wild-type AAVS1 |
| | | protospacer with PAM |
| 10 | CGGAACTCTGCCCTCTAACG | Primer 1 AAVS1-outsideF |
| 11 | CTGGGATACCCCGAAGAGTG | Primer 2 AAVS1-outsideR |
| 12 | CCTCTCCATCCTCTTGCTTTCTTTG | AAVS1 TIDE-4R |
| 13 | AACTGCTTCTCCTCTTGGGAAGT | AAVS1 TIDE-4F |
| 14 | GATGGGGGTGTTCTGCTGG | IntAmp1 |
| 15 | CGTAAACGGCCACAAGTTCA | IntAmp2 |
| 16 | GGGGCCACTAGGGAGAGGATAGG | AAVS1 DAP with PAM 1 |
| 17 | GGGGCCACTAGGGCCAGGATAGG | AAVS1 DAP with PAM 2 |
| 18 | GGGGCCACTAGGAACAGGATAGG | AAVS1 DAP with PAM 3 |
| 19 | GGGACCACTAGGGACAGGATAGG | AAVS1 DAP with PAM 4 |
| 20 | GGAGCCACTAGGGACAGGATAGG | AAVS1 DAP with PAM 5 |
| 21 | GAGGCCACTAGGGACAGGATAGG | AAVS1 DAP with PAM 6 |
| 22 | TGGGCCACTAGGGACAGGATAGG | AAVS1 DAP with PAM 7 |
| 23 | CAGGCCACTAGGGACAGGATAGG | AAVS1 DAP with PAM 8 |
| 24 | TAGGCCACTAGGGACAGGATAGG | AAVS1 DAP with PAM 9 |
| 25 | CTAGCCACTAGGGACAGGATAGG | AAVS1 DAP with PAM |
| | | 10 |
| 26 | TATGCCACTAGGGACAGGATAGG | AAVS1 DAP with PAM |
| | | 11 |
| 27 | GATGCCACTAGGGACAGGATAGG | AAVS1 DAP with PAM |
| | | 12 |
| 28 | GTAGCCACTAGGGACAGGATAGG | AAVS1 DAP with PAM |
| | | 13 |

What is claimed is:

1. A method for genome modification at a target locus in a hematopoietic stem cell (HSC), comprising:
(a) introducing a nuclease or nucleic acid encoding the nuclease into the HSC, wherein the target locus comprises a first recognition sequence for the nuclease;
(b) introducing a double-stranded donor nucleic acid into the HSC, wherein the double-stranded nucleic acid comprises an exogenous nucleic acid sequence and is configured to be inserted into the target locus by a homology-independent mechanism.

2. The method of claim 1, wherein the HSC is a long-term engrafting HSC (LT-HSC) or a SCID-repopulating cell.

3. The method of claim 1, wherein the HSC is characterized by the following markers: Lin⁻/CD34⁺/CD38⁻/CD90⁺/CD45RA⁻.

4. The method of claim 3, wherein Lin⁻ is characterized as one or more of CD235a⁻, CD41a⁻, CD3⁻, CD19⁻, CD14⁻, CD16⁻, CD20⁻, and CD56″.

5. The method of claim 4, wherein Lin⁻ is characterized as CD235a⁻/CD41a⁻/CD3″/CD19⁻/CD14⁻/CD16⁻/CD20⁻/CD56⁻.

6. A method for genome modification at a target locus in a quiescent T cell, comprising:
(a) introducing a nuclease or nucleic acid encoding the nuclease into the quiescent T cell, wherein the target locus comprises a first recognition sequence for the nuclease;
(b) introducing a double-stranded donor nucleic acid into the quiescent T cell, wherein the double-stranded nucleic acid comprises an exogenous nucleic acid sequence and is configured to be inserted into the target locus by a homology-independent mechanism.

7. The method of claim 6, wherein the quiescent T cell is a non-activated T cell.

8. A method for genome modification at a target locus in an HDR-deficient cell, comprising:
(a) introducing a nuclease or nucleic acid encoding the nuclease into the HDR-deficient cell, wherein the target locus comprises a first recognition sequence for the nuclease;
(b) introducing a double-stranded donor nucleic acid into the HDR-deficient cell, wherein the double-stranded nucleic acid comprises an exogenous nucleic acid sequence and is configured to be inserted into the target locus by a homology-independent mechanism, and
(c) culturing the HDR-deficient cell for a time sufficient for integration of the double-stranded donor nucleic acid into the target locus, wherein
steps (a), (b), and (c) are carried out such that the insertion efficiency for the double-stranded donor nucleic acid is at least about 4%.

9. The method of claim 8, wherein the insertion efficiency for the double-stranded donor nucleic acid is at least about 8%.

10. The method of any one of claims 1-9, wherein the double-stranded donor nucleic acid further comprises a second recognition sequence for the nuclease flanking a first end of the exogenous nucleic acid sequence.

11. The method of claim 10, wherein the double-stranded donor nucleic acid further comprises a third recognition sequence flanking a second end of the exogenous nucleic acid sequence.

12. The method of claim 10 or 11, wherein the double-stranded donor nucleic acid is cleaved at the second and/or third recognition sequence following introduction into the cell.

13. The method of any one of claims 1-12, wherein the double-stranded donor nucleic acid is configured such that insertion of the cleaved double-stranded donor nucleic acid into the target locus in a desired orientation does not create recognition sequences for the nuclease in the modified target locus and insertion of the cleaved double-stranded donor nucleic acid into the target locus in the other orientation creates a recognition sequence for the nuclease in the modified target locus.

14. The method of any one of claims 1-13, wherein the nuclease is an RNA-guided endonuclease (RGEN), each of the recognition sequences for the nuclease in the target locus and double-stranded donor nucleic acid is a protospacer sequence, and the method further comprises introducing into the cell one or more gRNAs targeting one or more of the protospacer sequences.

15. The method of claim 14, comprising introducing into the cell a gRNA comprising a spacer targeting the protospacers in the target locus and the donor nucleic acid.

16. The method of claim 15, wherein the protospacer in the target locus is in a forward orientation, the exogenous nucleic acid in the double-stranded donor nucleic acid is in a forward orientation, and the protospacers in the double-stranded donor nucleic acid are in a reverse orientation.

17. The method of claim 15 or 16, wherein the protospacers in the target locus and the donor nucleic acid are the same.

18. The method of claim 15 or 16, wherein at least one of the protospacers in the target locus and the donor nucleic acid is a delayed-action protospacer (DAP) incompletely matching the gRNA spacer.

19. The method of claim 18, wherein the DAP i) is shorter in length than the gRNA spacer by at least about 1 nucleotide; and/or ii) comprises at least about 1 nucleotide mismatch with the gRNA spacer.

20. The method of claim 18 or 19, wherein the protospacers in the donor nucleic acid are DAPs, and the protospacer in the target locus completely matches the gRNA spacer.

21. The method of claim 20, wherein the double-stranded donor nucleic acid comprises two DAPs flanking the exogenous nucleic acid sequence.

22. The method of claim 18 or 19, wherein the protospacers in the donor nucleic acid completely match the gRNA spacer, and the protospacer in the target locus is a DAP.

23. The method of any one of claims 14-22, wherein the RGEN is a Cas9 nuclease.

24. The method of any one of claims 14-23, comprising introducing into the cell a ribonucleoprotein (RNP) comprising the RGEN and the one or more gRNAs.

25. The method of any one of claims 14-23, comprising introducing into the cell an mRNA encoding the RGEN.

26. The method of any one of claims 1-25, wherein the double-stranded donor nucleic acid is a double-stranded virus genome.

27. The method of claim 26, wherein the double-stranded virus genome is an adenovirus genome, a lentivirus genome, or an adeno-associated virus (AAV) genome.

28. The method of claim 27, wherein the AAV genome is a self-complementary AAV (scAAV) genome.

29. The method of claim 28, wherein the scAAV genome is an scAAV6 genome.

30. The method of any one of claims 1-29, wherein the nuclease or nucleic acid encoding the nuclease is introduced into the cell before the donor nucleic acid is introduced into the cell.

31. The method of claim 30, wherein the nuclease or nucleic acid encoding the nuclease is introduced into the cell no more than 1 hour before the donor nucleic acid is introduced into the cell.

32. The method of claim 31, wherein the nuclease or nucleic acid encoding the nuclease is introduced into the cell no more than 5 minutes before the donor nucleic acid is introduced into the cell.

33. The method of any one of claims 1-32, wherein the cell is cultured under hypoxic conditions.

34. The method of any one of claims 1-33, wherein the cell is cultured no longer than about 48 hours prior to introducing the nuclease or nucleic acid encoding the nuclease and the donor nucleic acid into the cell.

35. The method of claim 34, wherein the cell is cultured no longer than about 24 hours prior to introducing the nuclease or nucleic acid encoding the nuclease and the donor nucleic acid into the cell.

36. The method of claim 35, wherein the cell is cultured no longer than about 2 hours prior to introducing the nuclease or nucleic acid encoding the nuclease and the donor nucleic acid into the cell.

37. The method of any one of claims 1-36, wherein the cell is cultured in the presence of a Notch ligand.

38. The method of claim 37, wherein the Notch ligand is a Delta-like Notch ligand (DLL), Jagged-1, Jagged-2, or a conjugate thereof.

39. The method of claim 38, wherein the Delta-like Notch ligand is DLL1, DLL3, or DLL4.

40. The method of claim 39, wherein the Notch ligand is Fc-DLL1, Fc-DLL3, Fc-DLL4, Fc-Jagged-1, or Fc-Jagged-2.

41. A method for engraftment in an individual of edited HSCs comprising an exogenous nucleic acid sequence inserted at a target locus, comprising:
(a) carrying out the method of any one of claims 1-40 on an input population of HSCs obtained from the individual to generate an output population of HSCs comprising a population of edited HSCs comprising the exogenous nucleic acid sequence inserted at the target locus; and
(b) administering the population of edited HSCs to the individual such that the edited HSCs are engrafted in the individual.

42. The method of claim 41, wherein the amount of engraftment of edited HSC in the individual is the same or greater than the amount of engraftment of corresponding edited HSCs prepared using a homology-dependent mechanism.

43. The method of claim 41 or 42, wherein the input population of HSCs obtained from the individual comprises a mixed population of HSCs comprising LT-HSCs and short-term engrafting HSCs (ST-HSCs), and wherein the population of edited HSCs that engrafted comprise edited LT-HSCs.

44. The method of any one of claims 41-43, wherein administering the population of edited HSCs to the individual comprises administering the output population of HSCs to the individual.

45. An engineered HSC prepared by a method for genome modification at a target locus in an HSC according to any one of claims 1-5 and 10-40.

46. An engineered quiescent T cell prepared by a method for genome modification at a target locus in a quiescent T cell according to any one of claims 6-7 and 10-40.

47. A method of treating a disease or condition in a subject, wherein the disease or condition is characterized by deficient expression of a functional protein, comprising administering to the subject an engineered cell according to claim 45 or 46, wherein the exogenous nucleic acid encodes a functional form of the protein that can be expressed in the engineered cell.

48. The method of claim 47, wherein the disease or condition is SCID, and wherein the exogenous nucleic acid comprises a functional form of a gene mutated in the individual involved in lymphoid development or lymphocyte proliferation and/or metabolism.

49. The method of claim 48, wherein the exogenous nucleic acid encodes a functional form of IL2Rg, RAG1, IL7R, ADA, or PNP.

50. The method of claim 47, wherein the disease or condition is Gaucher disease, Fabry disease, mucopolysaccharidosis types I-IX, or adrenoleukodystrophy.
